(19) United States
(12) Patent Application Publication
(10) Pub. No.: US 2022/0204495 A1
(43) Pub. Date: Jun. 30, 2022

(54) INHIBITING TRABID

(71) Applicants: Valo Health, Inc., Boston, MA (US); Integral Health Holdings, LLC, Boston, MA (US)

(72) Inventors: Anne-Marie CAMPBELL, Boston, MA (US); Lawrence MARCIN, Boston, MA (US); Katherine KAYSER-BRICKER, Boston, MA (US)

(21) Appl. No.: 17/608,392

(22) PCT Filed: May 2, 2020
(86) PCT No.: PCT/US2020/031214

§ 371 Date: Nov. 2, 2021

Related U.S. Application Data
(60) Provisional application No. 62/843,000, filed on May 3, 2019.
(60) Provisional application No. 62/850,609, filed on May 21, 2019.

(51) Int. Cl. C07D 417/06; C07D 277/56; C07D 277/46; C07D 403/06; C07D 413/06; C07D 413/10; C07D 413/14; C07D 261/04; C07D 261/18

(52) U.S. Cl. CPC C07D 417/06; C07D 277/56; C07D 277/46; C07D 403/06; C07D 261/18; C07D 413/10; C07D 413/14; C07D 261/04; C07D 413/06

(57) ABSTRACT

The present disclosure is directed to compounds of formulas (I)-(VII), which are useful as modulators of TRABID. The compounds are further useful in the inhibition of TRABID and the treatment of diseases or disorders associated with the inhibition of TRABID. For instance, the disclosure is concerned with compounds and compositions for inhibition of TRABID, methods of treating diseases associated with the inhibition of TRABID (e.g., autoimmune inflammatory diseases including, but not limited to, psoriasis), and methods of synthesis of these compounds.

### CROSS-REFERENCE TO RELATED APPLICATIONS

This application claims the benefit of U.S. Provisional Application No. 62/843,000, filed May 3, 2019; and U.S. Provisional Application No. 62/850,609, filed May 21, 2019; each of which is incorporated by reference herein in its entirety.

### TECHNICAL FIELD

The present disclosure relates to compounds useful for the inhibition of TRABID and methods of their preparation. Inhibitors of TRABID are useful compounds for the treatment of autoimmune inflammatory diseases including, but not limited to, psoriasis.

### BACKGROUND

TRABID belongs to the ovarian tumor protease (OTU) family of DUBs and was originally linked to the Wnt/β-catenin signaling pathway. More recently it has been found to be a regulator of IL-23 and IL-12 through deubiquitination of the demethylase Jmjd2d. Psoriasis is a chronic autoimmune disease that manifests as skin lesions (psoriatic plaques), as well as systemic inflammation and co-morbidities associated with systemic inflammation. The primary pathway of psoriasis pathogenesis initiates with activation of dendritic cells to produce IL-23 and IL-12, which leads to the differentiation of immune cells into Th17 and Th1, respectively. A cascade of cytokines and chemokines induces an inflammatory response involving recruitment of inflammatory cells, keratinocyte activation and proliferation, and melanocyte activation, eventually resulting in formation of skin plaques.

TRABID deficiency and TRABID deletion promote Jmjd2d degradation, leading to increased histone methylation at the promotor region of the IL-12b gene and repression of IL-12 and IL-23. In addition, IL-23 and IL-12 induction in activated dendritic cells are inhibited by deletion of Zranb1, the gene that encodes TRABID. Zranb1 deletion also disrupts T cell differentiation and protects mice from autoimmune inflammation. Levels of IL-12 and IL-23 are reduced, and the defect in T cell differentiation is rescued by exogenous IL-12 and IL-23. These findings support TRABID inhibition as an epigenetic mechanism for reducing IL-23 and IL-12 levels in the cell, and a potential therapeutic approach for disrupting the pro-inflammatory cascade associated with these cytokines.

IL-23 and IL-12 are heterodimeric proteins that have a p40 (IL-12/23p40) subunit in common and are differentiated by the IL-23p19 and IL-12p35 subunits, respectively. IL-23 and IL-12 signal through their heterodimeric receptor complexes where the common IL12/23p40 subunit binds at the IL-12Rβ1 receptor subunit while the differentiating IL23p19 and IL12p35 subunits bind at IL-23R and IL-12Rβ2 receptor subunits, respectively. Notably, the IL12B gene, which encodes the p40 subunit, has been identified as a psoriasis susceptibility gene. A specific polymorphism of the IL12B gene, rs32122217, is associated with psoriasis risk likely due to its regulation of IL-23 and IL-12 expression. IL23R, which codes for the IL-23 receptor, was also identified as a susceptibility gene. Notably mRNA levels in diseased skin show higher expression of IL-23p19 and IL-12/23p40 in psoriatic lesions compared to psoriatic non-lesional skin and normal skin, and higher in psoriatic non-lesional skin compared to normal skin. There are no differences in levels if IL-12p35mRNA across the three skin types, suggesting IL-23 has a greater role in the pathogenesis of psoriasis than IL-12.

Strong evidence linking IL-23 and IL-12 to moderate-to-severe psoriasis also emerges from clinical trials where IL-23 and IL-12 antibodies have demonstrated strong efficacy. Ustekinumab inhibits both IL-23 and IL-12 and is approved for treatment of plaque psoriasis. There are currently three monoclonal antibodies (mAb) that selectively target IL-23 and are approved (ie, guselkumab and tildrakizumab) or are in late stage clinical trials for treatment of moderate-to-severe psoriasis (ie, risankizumab). Notably, clinical evaluations with IL-23 antibodies showed a downregulation of ongoing Th17 responses and disease control even after clearance of the active substance. These clinical results are consistent with findings that human lesional psoriatic skin has increased levels of IL-23 and IL-12, and downstream cytokines, IL-17 and IL-22. Serum levels of these cytokines correlate with disease severity.

The pivotal role of IL-23 in psoriatic lesions has also been confirmed in animal models. IL-23 injections into mouse skin induce histological changes consistent with psoriatic lesional skin. These dermal changes are not observed in IL-17 or IL-22 knockout mice. Similarly, imiquimod-induced psoriasis in mice induces upregulation of the IL-23/Th17 axis; however, skin lesions are suppressed in mice deficient for a specific IL-23 subunit (ie, p19) or the IL-17 receptor A. Together the findings in mice suggest the IL-23 pathway is necessary for inflammatory effects related to psoriatic lesions.

Employing small molecule TRABID inhibitors to reduce levels of IL-23 and IL-12 and treat moderate-to-severe psoriasis offers a novel and alternative approach to the currently available mAb and receptor-specific antagonists that are typically used to treat autoimmune inflammatory diseases. The ability to target both cytokines is due to their similarity in structure and shared p40 subunit.

### SUMMARY

In one aspect, a compound of Formula (I) is disclosed:

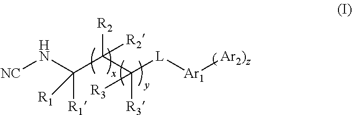

and pharmaceutically acceptable salts, hydrates, solvates, isomers, and tautomers thereof, wherein the substituents are variables are described later herein.

In another aspect, a method of treating, preventing, inhibiting, or eliminating a disease or disorder associated with the activity of TRABID in a patient is disclosed which includes administering to the patient in need thereof a therapeutically effective amount of the foregoing compounds, or pharmaceutical compositions thereof.

### DETAILED DESCRIPTION

The present disclosure relates to compounds that are capable of modulating the activity of TRABID. The disclosure features methods of treating, preventing, or ameliorating a disease or disorder in which TRABID plays a role by administering to a patient in need thereof a therapeutically effective amount of a compound of any one of formulas (I)-(VII), or a pharmaceutically acceptable salt thereof. The methods of the present disclosure can be used in the treatment of a variety of TRABID-dependent diseases and disorders by inhibiting the activity of TRABID. Inhibition of TRABID provides a novel approach to the treatment of autoimmune inflammatory diseases including, but not limited to, psoriasis.

### Definitions

The articles “a” and “an” are used in this disclosure to refer to one or more than one (e.g., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety can (but is not required to) be bonded to other substituents. Unless otherwise specifically defined, optional substituents bond to the chemical moiety with any chemically feasible regiochemistry and/or stereochemistry (where applicable). For instance, an alkyl group that is optionally substituted can be a fully saturated alkyl chain (e.g., a pure hydrocarbon). Alternatively, the same optionally substituted alkyl group can have substituents in place of one or more hydrogen atoms. For instance, it can be bonded, at any point along the chain, to any recited optional substituent. Thus, the term “optionally substituted” means that a given chemical moiety has the potential to contain other functional groups, but does not necessarily have any further functional groups. “Optionally substituted” as used herein also refers to substituted or unsubstituted whose meaning is described below.

As used herein, the term “substituted” means that the specified group or moiety bears one or more of the recited substituents, wherein the substituents may connect to the specified group or moiety at one or more positions. Unless otherwise specifically defined, substituents may be bonded to the chemical moiety with any chemically feasible regiochemistry and/or stereochemistry (where applicable).

As used herein, the term “unsubstituted” means that the specified group bears no substituents other than those illustrated in the formula which defines the structure.

As used herein, the term “aryl” refers to monocyclic, aromatic hydrocarbon groups that have one aromatic ring having a total of 5 to 14 ring atoms, such as phenyl.

As used herein, the term “bicyclic aryl” refers to bicyclic, aromatic hydrocarbon groups that have one aromatic ring having a total of 5 to 14 ring atoms, such as naphthalenyl and indenyl.

As used herein, the term “heteroaryl” refers to a monocyclic aromatic radical of 5 to 14 ring atoms, containing one or more ring heteroatoms selected from the group consisting of N, O, and S, the remaining ring atoms being C. Examples include, but are not limited to, furyl, thienyl, pyrrolyl, pyridinyl, pyrazolyl, pyrimidinyl, imidazolyl, isoxazolyl, oxazolyl, oxadiazolyl, pyrazinyl, thiophen-2-yl, isothiazolyl, thiazolyl, thiadiazole, triazolyl, triazinyl.

As used herein, the term “bicyclic heteroaryl” means a bicyclic aromatic radical, containing one or more ring heteroatoms selected from the group consisting of N, O, and S, the remaining ring atoms being C. Examples include, but are not limited to, indolyl, quinolyl, benzopyranyl, indazolyl, benzimidazolyl, thieno[3,2-b]thiophene, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, 6,7-dihydro-4H-thieno[3.2-c]pyran.

As used herein, the terms “halogen” or “halo” refer to fluorine, chlorine, bromine, or iodine.

As used herein, the term “(C₁-C₄) alkyl” refers to a straight or branched chain saturated hydrocarbon containing 1-4 carbon atoms. Examples of a (C₁-C₄) alkyl group include, but are not limited to, methyl, ethyl, propyl, butyl, isopropyl, isobutyl, sec-butyl, tert-butyl. “C₁ alkyl” refers to an alkyl chain containing 1 carbon atom, e.g. methyl. “C₂ alkyl” refers to an alkyl chain containing 2 carbon atoms, e.g. ethyl. “C₃ alkyl” refers to an alkyl chain containing 3 carbon atoms, e.g. propyl or isopropyl. “C₄ alkyl” refers to an alkyl chain containing 4 carbon atoms, e.g. butyl, isobutyl, sec-butyl, or tert-butyl.

As used herein, the term “(C₃-C₆) cycloalkyl” refers to a monocyclic saturated ring containing 3-6 carbon atoms. Examples of cycloalkyl groups include, without limitations, cyclopropyl, cyclobutyl, cyclopentyl, cyclohexyl. “C₃ cycloalkyl” refers to a cycloalkyl containing 3 carbon atoms, e.g. cyclopropyl. “C₄ cycloalkyl” refers to a cycloalkyl containing 4 carbon atoms, e.g. cyclobutyl. “C₅ cycloalkyl” refers to a cycloalkyl containing 5 carbon atoms, e.g. cyclopentyl. “C₆ cycloalkyl” refers to a cycloalkyl containing 6 carbon atoms, e.g. cyclohexyl.

As used herein, the term “heterocyclyl” refers to a monocyclic or bicyclic ring containing a total of 3 to 10 carbon and heteroatoms taken from oxygen, nitrogen, or sulfur, where such rings are either saturated or partially unsaturated. The term “heterocyclyl” encompasses monocyclic heterocyclyls, and bicyclic heterocyclyls including spriocyclic heterocyclyls and fused heterocyclyls.

The terms “heterocyclyl” and “heterocycloalkyl” are used interchangeably herein.

Examples of monocyclic heterocyclyl rings include, but are not limited to, oxetanyl, azetidinyl, tetrahydrofuranyl, tetrahydropyranyl, pyrrolidinyl, oxazolidinyl, and thiazolidinyl.

As used herein, the term “spirocyclyl” refers to a bicyclic ring system having 6-12 atoms, including carbon atoms and optionally heteroatoms taken from oxygen, nitrogen, or sulfur, wherein the rings are connected to one another through a single atom referred to as the spiro atom. The rings can be different in size and nature, or identical in size and nature. Spirocyclyls can be described in terms of their two constituent rings: for instance, the 7-membered spirocyclyl 5-azaspiro[2.4]heptanyl can be described as including a cyclopropyl and a pyrollidinyl. Examples of spirocyclyls wherein all ring atoms are carbon include, but are not limited to, spirohexanyl, spiroheptanyl, spirooctanyl, spirononanyl, spirodecanyl, spiroundecanyl, and spirododecanyl. Examples wherein the spirocyclyl contains at least one heteroatom include but are not limited to 5-azaspiro[2.4]heptanyl, 6-azaspiro[3.4]octanyl, 7-azaspiro[3.5]nonanyl.

As used herein, the term “fused heterocyclyl” refers to a bicyclic ring system having 6-12 atoms including carbon atoms and heteroatoms taken from oxygen, nitrogen, or sulfur, wherein the rings are connected to one another through two atoms covalently bonded to one another. The rings can be different in size and nature, or identical in size and nature. Fused heterocyclyls can be described in terms of their two constituent rings: for instance, the 6-membered fused heterocyclyl 3-azabicyclo[3.1.0]hexanyl includes a 3-membered cycloalkyl (cyclopropyl) fused to a 5-membered heterocyclyl (pyrollidinyl). Other examples of fused heterocyclyls include but are not limited to 3-azabicyclo[3.2.0]heptanyl, 3-thiabicyclo[3.2.0]heptanyl, 2-azabicyclo[4.1.0]heptanyl, and so forth.

As used herein, when an atom, a group, or a substituent is said to “form a heterocycloalkyl,” this is understood to be inclusive of spirocyclyls and fused heterocyclyls in which the atom, group, or substituent forms a ring of the spirocyclyl or fused heterocyclyl which does not contain a heteroatom. For instance, the 6-membered fused heterocyclyl 3-azabicyclo[3.1.0]hexanyl includes a 3-membered cycloalkyl (cyclopropyl) fused to a 5-membered heterocyclyl (pyrollidinyl). As used herein, any carbon of the 3-membered cycloalkyl will be considered to be part of a heterocycloalkyl rather than a cycloalkyl because the smaller cycloalkyl is a constituent of the larger fused heterocyclyl.

As used herein, the term “isomer” refers to compounds that have the same composition and molecular weight but differ in physical and/or chemical properties. The structural difference may be in constitution (e.g., geometric isomers) or in the ability to rotate a plane of polarized light (stereoisomers). With regard to stereoisomers, the compounds of Formula (I) may have one or more asymmetric carbon atoms and may occur as racemates, racemic mixtures or as individual enantiomers or diastereomers.

The compounds of formula (I)-(IX), unless otherwise indicated, may contain one or more stereocenters, and, therefore, exist in different stereoisomeric forms. It is intended that unless otherwise indicated all stereoisomeric forms of the compounds of formulas (I)-(IX), including enantiomeric forms (which may exist even in the absence of asymmetric carbons), rotameric forms, atropisomers, and diastereomeric forms, as well as mixtures thereof, including racemic mixtures, form part of the present disclosure. In addition, the present disclosure embraces all geometric and positional isomers. For example, if a compound of any one of formulas (I)-(IX) incorporates a double bond or a fused ring, both the cis- and trans-forms, as well as mixtures, are embraced within the scope of the disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry. Individual stereoisomers of the compounds of the disclosure may be, for example, substantially free of other isomers, or may be admixed, for example, as racemates or with all other, or other selected, stereoisomers. In some embodiments of the disclosure, the compounds of formula (I)-(IX) are enantiomers. In some embodiments, the compounds are the (S)-enantiomer. In other embodiments, the compounds are the (R)-enantiomer. In some embodiments, the compounds of formulas (I)-(IX) may be (+) or (−) enantiomers.

In addition, unless otherwise indicated, the present disclosure embraces all geometric and positional isomers (such as, for example, 4-pyridyl and 3-pyridyl). For example, if a compound of the disclosure incorporates a double bond or a fused ring, both the cis- and trans-forms, as well as mixtures, are embraced within the scope of the disclosure. If the compound contains a double bond, the substituent may be in the E or Z configuration, unless otherwise indicated. If the compound contains a disubstituted cycloalkyl, the cycloalkyl substituent may have a cis- or trans configuration, unless otherwise indicated.

Compounds of the disclosure, and pharmaceutically acceptable salts and stereoisomers, thereof may exist in their tautomeric form (for example, as an amide or imino ether). Moreover, all keto-enol and imine-enamine forms of the compounds are included in the disclosure. All such tautomeric forms are contemplated herein as part of the present disclosure.

The use of the terms “salt” and the like, is intended to equally apply to the salt of enantiomers, stereoisomers, rotamers, tautomers, positional isomers, and racemates of the inventive compounds.

The disclosure also includes pharmaceutical compositions comprising an effective amount of a disclosed compound and a pharmaceutically acceptable carrier.

The term “pharmaceutical composition” as used herein, refers to a composition in which individual components or ingredients are themselves pharmaceutically acceptable, e.g., where oral administration is foreseen, acceptable for oral use; where topical administration is foreseen, topically acceptable; and where intravenous administration is foreseen, intravenously acceptable.

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates where water is the solvent are typically referred to as hydrates. Hydrates include compositions containing stoichiometric amounts of water, as well as compositions containing variable amounts of water.

“Pharmaceutically acceptable salts” are well known in the art. For example, S. M. Berge et al., describe pharmaceutically acceptable salts in detail in J. Pharmaceutical Sciences, 1977, 66, 1-19, incorporated herein by reference. Representative pharmaceutically acceptable salts include, e.g., water-soluble and water-insoluble salts, such as acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. The compounds of formulas (I)-(IX) may form salts which are also within the scope of this disclosure. Reference to a compound of Formula I herein is understood to include reference to salts thereof, unless otherwise indicated.

When compounds of the present invention contain relatively acidic functionalities, base addition salts can be obtained by contacting the neutral form of such compounds with a sufficient amount of the desired base, either neat or in a suitable inert solvent. Salts derived from pharmaceutically-acceptable inorganic bases include aluminum, ammonium, calcium, copper, iron(III), iron(II), lithium, magnesium, manganese, potassium, sodium, zinc, and the like. Salts derived from pharmaceutically-acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When compounds of the present invention contain relatively basic functionalities, acid addition salts can be obtained by contacting the neutral form of such compounds with a sufficient amount of the desired acid, either neat or in a suitable inert solvent. Acids suitable for the preparation of pharmaceutically acceptable acid addition salts include acetic, ascorbic, benzenesulfonic, benzoic, camphorsulfonic, citric, ethanesulfonic, fumaric, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, tartaric, p-toluenesulfonic, and the like.

The compounds of formulas (I)-(IX) may form acid addition salts or base addition salts, which may be pharmaceutically acceptable salts. The disclosure also includes pharmaceutical compositions comprising one or more compounds as described herein, or a pharmaceutically acceptable salt thereof, and a pharmaceutically acceptable carrier. In some embodiments, pharmaceutical compositions reported herein can be provided in a unit dosage form (e.g., capsule, tablet or the like). In some embodiments, pharmaceutical compositions reported herein can be provided in an oral dosage form. In some embodiments, an oral dosage form of a compound of any one of formulas (I)-(IX) can be a capsule. In some embodiments, an oral dosage form of a compound of any one of formulas (I)-(IX) is a tablet. In some embodiments, an oral dosage form comprises one or more fillers, disintigrants, lubricants, glidants, anti-adherents and/or anti-statics. In some embodiments, an oral dosage form is prepared via dry blending. In some embodiments, an oral dosage form is a tablet and is prepared via dry granulation.

A TRABID Inhibitor Compound of the present disclosure can be dosed at a therapeutically effective level.

A “patient” or “subject” is a mammal, e.g., a human, mouse, rat, guinea pig, dog, cat, horse, cow, pig, or non-human primate, such as a monkey, chimpanzee, baboon, or rhesus.

As used herein, the term “therapeutically effective amount” refers to that amount of the therapeutic agent sufficient to result in amelioration of one or more symptoms of a disorder, or prevent advancement of a disorder, or cause regression of the disorder.

The term “carrier”, as used in this disclosure, encompasses carriers, excipients, and diluents and means a material, composition or vehicle, such as a liquid or solid filler, diluent, excipient, solvent, or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound, a pharmaceutically acceptable salt of a disclosed compound or a composition to a subject, a pharmaceutically acceptable salt of a compound, or a composition to a subject, which can form an equivalent amount of active compound within the subject's body.

Compositions in accordance with the present invention may be administered in any appropriate manner, e.g., oral or buccal administration. When orally administered, the compound of formula I may be prepared as a mixture with excipients suitable for the manufacture of oral dosage forms such as tablets, in a solution or suspension, in hard or soft encapsulated form including gelatin encapsulated form, sachet, or lozenge. Suspensions for oral administration may be prepared according to any method known to those skilled in the art. For example, suspensions may be oily suspensions in which a compound of any one of formulas (I)-(IX) is suspended in a liquid suspension comprising, for example, vegetable oils such as olive oil, sesame oil, or coconut oil. The liquid suspension may also contain mineral oil.

Compositions may also be administered topically, e.g., for application to the skin, for example in the form of a cream, paste, lotion, gel, ointment, poultice, cataplasm, plaster, dermal patch or the like, or for ophthalmic application, for example in the form of an eye drop, -lotion or -gel formulation.

Compositions may also be administered parenterally, e.g., intravenous. Intravenous forms include, but are not limited to, bolus and drip injections. In some embodiments, the intravenous dosage forms are sterile or capable of being sterilized prior to administration to a subject since they typically bypass the subject's natural defenses against contaminants. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Readily flowable forms, for example solutions, emulsions and suspensions, may also be employed e.g., for intralesional injection, or may be administered rectally, e.g., as an enema or suppository, or intranasal administration, e.g., as a nasal spray or aerosol. Macrocrystalline powders may be formulated for inhalation, e.g., delivery to the nose, sinus, throat or lungs. Transdermal compositions/devices and pessaries may also be employed for delivery of the compounds of the invention. The compositions may additionally contain agents that enhance the delivery of the compounds having Formula I (or other active agents), e.g., liposomes, polymers or co-polymers (e.g., branched chain polymers). Preferred dosage forms of the present invention include oral dosage forms and intravenous dosage forms.

The pharmaceutical compositions of the present invention may further comprise one or more additives. Additives that are well known in the art include, e.g., detackifiers, anti-foaming agents, buffering agents, antioxidants (e.g., ascorbic acid, ascorbyl palmitate, sodium ascorbate, butylated hydroxyanisole (BHA), butylated hydroxytoluene (BHT), propyl gallate, malic acid, fumaric acid, potassium metabisulfite, sodium bisulfite, sodium metabisulfite, and tocopherols, e.g., α-tocopherol (vitamin E)), preservatives, chelating agents, viscomodulators, tonicifiers, flavorants, colorants, odorants, opacifiers, suspending agents, binders, fillers, plasticizers, lubricants, and mixtures thereof. The amounts of such additives can be readily determined by one skilled in the art, according to the particular properties desired, and can be formulated such that compounds having Formula I are stable, e.g., not reduced by antioxidant additives.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropyl ethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents may not be required. Use of thickening agents is, on the other hand, indicated, e.g., where topical application is foreseen.

Although the dosage of a compound of any one of formulas (I)-(IX) will vary according to the activity and/or toxicity of the particular compound, the condition being treated, and the physical form of the pharmaceutical composition being employed for administration, it may be stated by way of guidance that a dosage selected in the range from 0.01 to 2000 mg/kg of body weight per day, or 0.1 to 1500 mg/kg, or 1 to 1000 mg/kg, will often be suitable. Those of ordinary skill in the art are familiar with methods for determining the appropriate dosage.

Novel TRABID inhibitors are provided. Unless otherwise indicated “TRABID Inhibitor Compound” as used herein refers to a compound having a detectable IC50 value of 10 micromolar or lower, when tested according to the TRABID inhibition biochemical assay of Example 21 described hereafter.

Unless otherwise indicated herein, all isomeric forms of specified chemical compounds are provided by the present disclosure, including mixtures thereof. All tautomeric forms are also intended to be included.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of formulas (I)-(VII) may be atropisomers (e.g., substituted biaryls) and are considered as part of this disclosure. Enantiomers can also be separated by use of a chiral HPLC column.

A TRABID Inhibitor Compound of the present disclosure can be dosed at a therapeutically effective level.

### Compounds of the Disclosure

The present disclosure relates to compounds, or pharmaceutically acceptable salts, hydrates, solvates, tautomers, and isomers thereof, capable of modulating TRABID, which are useful for the treatment of diseases and disorders associated with modulation of TRABID. The disclosure further relates to compounds, or pharmaceutically acceptable salts and isomers thereof, which are useful for inhibiting TRABID.

In another aspect, a compound of Formula (I) is disclosed:

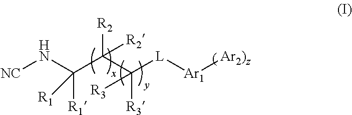

and pharmaceutically acceptable salts, hydrates, solvates, isomers, and tautomers thereof, wherein: z is zero or one, and wherein:
(A) when z is one:

R₁ and R₁′ are each independently hydrogen, (C₁-C₄) alkyl, or together with one of R₂, R₃, or R₄, form a heterocycloalkyl;

R₂ and R₂′ are each independently hydrogen, (C₁-C₄) alkyl, or together with one of R₁, R₃, or R₄, form a cycloalkyl or heterocycloalkyl;

R₃ and R₃′ are each independently hydrogen, (C₁-C₄) alkyl, or together with one of R₁, R₂, or R₄, form a cycloalkyl or heterocycloalkyl;

x and y are each independently zero or one;

L is —C(O)NR₄— or —NR₄C(O)—;

R₄ is hydrogen, (C₁-C₆) alkyl, or together with any one of R₁, R₂, or R₃, form a heterocycloalkyl;

Ar₁ is unsubstituted heteroaryl with up to two heteroatoms independently selected from the group consisting of N, O, and S;

Ar₂ is independently an aryl or heteroaryl optionally substituted with one or more R₁₀, R₁₂, or —OR₁₂, or together with Ar₁, form a fused bicyclic (C₈-C₁₀) aryl or heteroaryl optionally substituted with one or more R₁₁;

each R₁₀ is independently halogen, (C₁-C₆) alkyl, (C₁-C₆) cycloalkyl, (C₁-C₆) alkoxy, aryloxy, or aryl or heteroaryl optionally substituted with one or more R₁₁;

each R₁₁ is independently hydroxyl or halogen; and

R₁₂ is aryl or heteroaryl, optionally substituted with one or more R₁₀;

(1) wherein, when L is —C(O)NR₄—,

x is one;

R₁, R₁′, R₂′, and R₄ are each hydrogen;

R₂ is hydrogen or (C₁-C₄) alkyl;

R₃, if present, is hydrogen or (C₁-C₄) alkyl;

R₃′, if present, is hydrogen; and

no combination of R₁, R₁′, R₂, R₂′, R₃, R₃′, and R₄ forms a cycloalkyl or heterocycloalkyl;

(2) wherein, when L is —NR₄C(O)—,

(a) when an R₁₀ or R₁₂ is heteroaryl substituted with alkyl, and at least two of R₁, R₁′, R₂, R₂′, R₃, R₃′, and R₄ combine to define a spirocyclyl comprising (i) a pyrrolidinyl and a cyclobutyl having a carbon atom as a spiro atom or (ii) an azetidinyl and a cyclobutyl having a carbon atom as a spiro atom, the spiro atom is not adjacent a nitrogen of the pyrrolidinyl or azetidinyl;

(b) when Ar₂ is substituted with more than one halogen, and R₄ forms a 5- or 6-membered heterocyclyl with another substituent, R₁ and R₁′ are each hydrogen;

(c) when Ar₂ is unsubstituted phenyl, and one of R₂, R₃, and R₄ forms a cyclobutyl or a spirocyclyl which includes a cyclobutyl, R₁ and R₁′ are each hydrogen;

(B) when z is zero:

R₁ and R₁′ are each independently hydrogen, (C₁-C₄) alkyl, or together with one of R₂, R₃, or R₄, form a cycloalkyl or heterocycloalkyl;

R₂ and R₂′ are each independently hydrogen, (C₁-C₄) alkyl, or together with one of R₁, R₃, or R₄, form a cycloalkyl or heterocycloalkyl;

R₃ and R₃′ are each independently hydrogen, (C₁-C₄) alkyl, or together with one of R₁, R₂, or R₄, form a cycloalkyl or heterocycloalkyl;

x and y are each independently zero or one;

L is —C(O)NR₄— or —NR₄C(O)—;

R₄ is hydrogen, (C₁-C₆) alkyl, or together with any one of R₁, R₂, or R₃, form a heterocycloalkyl; and

Ar1 is independently an aryl or heteroaryl substituted with one aryloxy.

In one aspect, compounds of formula (Ia) are disclosed:

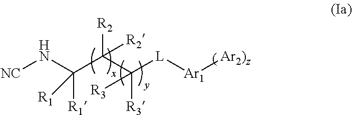

and pharmaceutically acceptable salts, hydrates, solvates, isomers, and tautomers thereof, wherein:

R₁ and R₁′ are each independently hydrogen, (C₁-C₄) alkyl, or together with one of R₂, R₃, or R₄, form a cycloalkyl or heterocycloalkyl;

R₂ and R₂′ are each independently hydrogen, (C₁-C₄) alkyl, or together with one of R₁, R₃, or R₄, form a cycloalkyl or heterocycloalkyl;

R₃ and R₃′ are each independently hydrogen, (C₁-C₄) alkyl, or together with one of R₁, R₂, or R₄, form a cycloalkyl or heterocycloalkyl;

x, y, and z are each independently zero or one;

L is —C(O)NR₄— or —NR₄C(O)—;

R₄ is hydrogen, (C₁-C₆) alkyl, or together with any one of R₁, R₂, or R₃, form a heterocycloalkyl;

Ar₁ is independently an aryl or heteroaryl optionally substituted with one or more R₁₀, or together with Ar₂, form a fused bicyclic (C₈-C₁₀) aryl or heteroaryl optionally substituted with one or more R₁₁;

Ar₂ is independently an aryl or heteroaryl optionally substituted with one or more R₁₀, R₁₂, or —OR₁₂, or together with Ar₁, form a fused bicyclic (C₈-C₁₀) aryl or heteroaryl optionally substituted with one or more R₁₁;

each R₁₀ is independently halogen, (C₁-C₆) alkyl, (C₁-C₆) cycloalkyl, (C₁-C₆) alkoxy, aryloxy, or aryl or heteroaryl optionally substituted with one or more R₁₁;

each R₁₁ is independently hydroxyl, halogen, or cyano; and

R₁₂ is aryl or heteroaryl, optionally substituted with one or more R₁₀.

In another embodiment, in a compound of Formula (I), z is zero, and:

R₁ and R₁′ are each independently hydrogen, (C₁-C₄) alkyl, or together with one of R₂, R₃, or R₄, form a cycloalkyl or heterocycloalkyl;

R₂ and R₂′ are each independently hydrogen, (C₁-C₄) alkyl, or together with one of R₁, R₃, or R₄, form a cycloalkyl or heterocycloalkyl;

R₃ and R₃′ are each independently hydrogen, (C₁-C₄) alkyl, or together with one of R₁, R₂, or R₄, form a cycloalkyl or heterocycloalkyl;

x, and y are each independently zero or one;

L is —C(O)NR₄— or —NR₄C(O)—;

R₄ is hydrogen, (C₁-C₆) alkyl, or together with any one of R₁, R₂, or R₃, form a heterocycloalkyl; and

Ar₁ is independently an aryl or heteroaryl substituted with one aryloxy.

In some embodiments in a compound of Formula (I), z is zero, and the compound is a compound of formula (VIII):

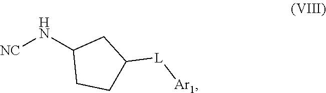

wherein: L is —C(O)NR₄— or —NR₄C(O)—; R₄ is hydrogen or (C₁-C₆) alkyl;

Ar₁ is independently an aryl or heteroaryl optionally substituted with one or more R₁₀;

when L is —NR₄C(O)—, each R₁₀ is independently halogen, (C₁-C₆) alkyl, (C₃-C₆) cycloalkyl, (C₁-C₆) alkoxy, aryloxy, or aryl or heteroaryl optionally substituted with one or more R₁₁, each R₁₁ being independently selected from hydroxyl, halogen, or cyano; and

when L is —C(O)NR₄—, each R₁₀ is independently halogen, (C₁-C₆) alkoxy, aryloxy, or aryl or heteroaryl optionally substituted with one or more R₁₁, each R₁₁ being independently selected from hydroxyl, halogen, or cyano.

In some embodiments, in a compound of Formula (I), z is one, and:

R₁ and R₁′ are each independently hydrogen, (C₁-C₄) alkyl, or together with one of R₂, R₃, or R₄, form a heterocycloalkyl;

R₂ and R₂′ are each independently hydrogen, (C₁-C₄) alkyl, or together with one of R₁, R₃, or R₄, form a cycloalkyl or heterocycloalkyl;

R₃ and R₃′ are each independently hydrogen, (C₁-C₄) alkyl, or together with one of R₁, R₂, or R₄, form a cycloalkyl or heterocycloalkyl;

x and y are each independently zero or one;

L is —C(O)NR₄— or —NR₄C(O)—;

R₄ is hydrogen, (C₁-C₆) alkyl, or together with any one of R₁, R₂, or R₃, form a heterocycloalkyl;

Ar1 is unsubstituted heteroaryl with up to two heteroatoms independently selected from the group consisting of N, O, and S;

Ar₂ is independently an aryl or heteroaryl optionally substituted with one or more R₁₀, R₁₂, or —OR₁₂, or together with Ar1, form a fused bicyclic (C₈-C₁₀) aryl or heteroaryl optionally substituted with one or more R₁₁;

each R₁₀ is independently halogen, (C₁-C₆) alkyl, (C₁-C₆) cycloalkyl, (C₁-C₆) alkoxy, aryloxy, or aryl or heteroaryl optionally substituted with one or more R₁₁;

each R₁₁ is independently hydroxyl or halogen; and

R₁₂ is aryl or heteroaryl, optionally substituted with one or more R₁₀; wherein, when L is —C(O)NR₄—,

x is one;

R₁, R₁′, R₂′, and R₄ are each hydrogen;

R₂ is hydrogen or (C₁-C₄) alkyl;

R₃, if present, is hydrogen or (C₁-C₄) alkyl;

R₃′, if present, is hydrogen; and

no combination of R₁, R₁′, R₂, R₂′, R₃, R₃′, and R₄ forms a cycloalkyl or heterocycloalkyl; and

wherein, when L is —NR₄C(O)—,

when an R₁₀ or R₁₂ is heteroaryl substituted with alkyl, and at least two of R₁, R₂, R₃, and R₄ combine to define a spirocyclyl comprising a pyrrolidinyl and a cyclobutyl or an azetidinyl and a cyclobutyl having a carbon atom as a spiro atom, the spiro atom is not adjacent a nitrogen of the pyrrolidinyl or azetidinyl; and

when Ar₂ is substituted with more than one halogen, and R₄ forms a 5- or 6-membered heterocyclyl with another substituent, R₁ and R₁′ are each hydrogen; and

when Ar₂ is unsubstituted phenyl, and one of R₂, R₃, and R₄ forms a cyclobutyl or a spirocyclyl which includes cyclobutyl, R₁ and R₁′ are each hydrogen.

In some embodiments, Ar₁ is selected from the group consisting of: pyrazolyl, thiazolyl, and isoxazolyl.

In some embodiments, the compound of formula (I) is further given by formula (II):

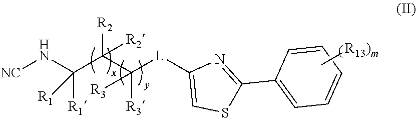

and pharmaceutically acceptable salts, hydrates, solvates, isomers, and tautomers thereof, wherein: L is —NR₄C(O)—, R₁₃ is selected from halogen, (C₁-C₄) alkoxy, and aryloxy, and m is 1 or 2.

In some embodiments, R₁₃ is selected from —Cl, —OCH₃, and —OC₆H5.

In some embodiments, the compound of formula (I) is further given by formula (III):

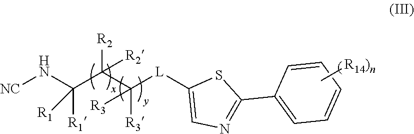

and pharmaceutically acceptable salts, hydrates, solvates, isomers, and tautomers thereof, wherein:
R₁₄ is (C₁-C₄) alkoxy or aryloxy, and n is 0 or 1.

In some embodiments, R₁₄ is —OCH₃ or —OC₆H5.

In some embodiments, the compound of formula (I) is further given by formula (IV):

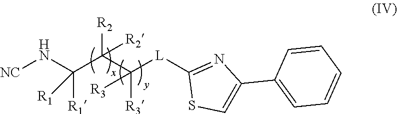

and pharmaceutically acceptable salts, hydrates, solvates, isomers, and tautomers thereof, wherein L is —NR₄C(O).

In some embodiments, the compound of formula (I) is further given by formula (V):

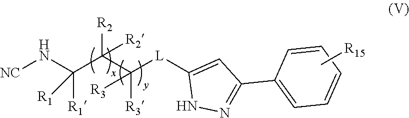

and pharmaceutically acceptable salts, hydrates, solvates, isomers, and tautomers thereof, wherein: L is —NR₄C(O), and R₁₅ is (C₁-C₄) alkyl or (C₁-C₄) alkoxy.

In some embodiments, R₁₅ is methyl or —OCH₃.

In some embodiments, the compound of formula (I) is further given by formula (VI):

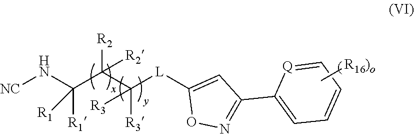

and pharmaceutically acceptable salts, hydrates, solvates, isomers, and tautomers thereof, wherein: Q is N or CH, R₁₆ is (C₁-C₄) alkoxy or heteroaryl substituted with (C₁-C₄) alkyl, and o is 0 or 1.

In some embodiments, R₁₆ is —OCH₃ or pyrazolyl substituted with one methyl.

Non-limiting, specific embodiments of the TRABID inhibitor compounds are shown below in Table B. In some embodiments, the compound of Formula (I) is a compound shown in Table B, or pharmaceutically acceptable salt, hydrate, solvate, isomer, or tautomer thereof.

In some embodiments, R₁ is hydrogen. In some embodiments, R₁ forms a heterocyclyl with R₄. In some embodiments, the heterocyclyl including R₁ is a spirocyclyl. In some embodiments, the spirocyclyl including R₁ in a 7-membered spirocyclyl, such as one which includes a 3-membered cycloalkyl and a 5-membered heterocyclyl, such as 5-azaspiro[2.4]heptanyl. In some embodiments, the spirocyclyl including R₁ is an 8-membered spirocyclyl, such as one which includes a 4-membered cycloalkyl and a 5-membered heterocyclyl, such as 6-azaspiro[3.4]octanyl. In some embodiments, the spirocyclyl including R₁ is a 9-membered spirocyclyl, such as one which includes a 4-membered cycloalkyl and a 6-membered heterocyclyl, such as 7-azaspiro[3.5]nonanyl. In some embodiments, the heterocyclyl including R₁ is a fused heterocyclyl. In some embodiments, the fused heterocyclyl including R₁ is bicyclic. In some embodiments, the fused heterocyclyl including R₁ is a 6-membered fused heterocyclyl, such as one which includes a 3-membered cycloalkyl fused to a 5-membered heterocyclyl, such as 3-azabicyclo[3.1.0]hexanyl.

In some embodiments, R₁′ is hydrogen.

In some embodiments, R₂ is hydrogen. In some embodiments, R₂ is (C₁-C₄) alkyl. In some embodiments, R₂ is methyl. In some embodiments, R₂ forms a cycloalkyl with R₃. In some embodiments, the cycloalkyl including R₂ is cyclobutyl. In some embodiments, R₂ forms a heterocyclyl with R₄. In some embodiments, the heterocyclyl including R₂ is a 4- to 6-membered heterocyclyl. In some embodiments, the heterocyclyl including R₂ is a 4- to 6-membered heterocyclyl which is a single ring, such as azetidinyl, morpholinyl, pyrrolidinyl, or piperidinyl. In some embodiments, the heterocyclyl including R₂ is a fused heterocyclyl. In some embodiments, the fused heterocyclyl including R₂ is bicyclic. In some embodiments, the fused heterocyclyl including R₂ is a 6-membered fused heterocyclyl, such as one which includes a 3-membered cycloalkyl fused to a 5-membered heterocyclyl, such as 3-azabicyclo[3.1.0]hexanyl.

In some embodiments, R₂′ is hydrogen. In some embodiments, R₂ is (C₁-C₄) alkyl. In some embodiments, R₂ is methyl.

In some embodiments, R₃ is hydrogen. In some embodiments, R₃ is (C₁-C₄) alkyl. In some embodiments, R₃ is methyl. In some embodiments, R₃ forms a cycloalkyl with R₂. In some embodiments, the cycloalkyl including R₃ is cyclobutyl.

In some embodiments, R₃′ is hydrogen. In some embodiments, R₃′ is (C₁-C₄) alkyl. In some embodiments, R₃′ is methyl.

In some embodiments, L is C(O)NR₄. In some embodiments, L is NR₄C(O).

In some embodiments, R₄ is hydrogen. In some embodiments, R₄ is (C₁-C₄) alkyl. In some embodiments, R₄ is methyl. In some embodiments, R₄ forms a heterocyclyl with R₁. In some embodiments, the heterocyclyl including R₄ is a spirocyclyl. In some embodiments, the spirocyclyl including R₄ in a 7-membered spirocyclyl, such as one which includes a 3-membered cycloalkyl and a 5-membered heterocyclyl, such as 5-azaspiro[2.4]heptanyl. In some embodiments, the spirocyclyl including R₄ is an 8-membered spirocyclyl, such as one which includes a 4-membered cycloalkyl and a 5-membered heterocyclyl, such as 6-azaspiro[3.4]octanyl. In some embodiments, the spirocyclyl including R₄ is a 9-membered spirocyclyl, such as one which includes a 4-membered cycloalkyl and a 6-membered heterocyclyl, such as 7-azaspiro[3.5]nonanyl. In some embodiments, the heterocyclyl including R₄ is a fused heterocyclyl. In some embodiments, the fused heterocyclyl including R₄ is bicyclic. In some embodiments, the fused heterocyclyl including R₄ is a 6-membered fused heterocyclyl, such as one which includes a 3-membered cycloalkyl fused to a 5-membered heterocyclyl, such as 3-azabicyclo[3.1.0]hexanyl. In some embodiments, R₄ forms a heterocyclyl with R₂. In some embodiments, the heterocyclyl including R₄ is a 4- to 6-membered heterocyclyl. In some embodiments, the heterocyclyl including R₄ is a 4- to 6-membered heterocyclyl which is a single ring, such as azetidinyl, morpholinyl, pyrrolidinyl, or piperidinyl. In some embodiments, the heterocyclyl including R₄ is a fused heterocyclyl. In some embodiments, the fused heterocyclyl including R₄ is bicyclic. In some embodiments, the fused heterocyclyl including R₄ is a 6-membered fused heterocyclyl, such as one which includes a 3-membered cycloalkyl fused to a 5-membered heterocyclyl, such as 3-azabicyclo[3.1.0]hexanyl.

In some embodiments, Ar₁ is an aryl or a heteroaryl. In some embodiments, Ar₁ is a 5-6 membered heteroaryl having 1 or 2 heteroatoms independently selected from N, O, and S. In some embodiments, Ar₁ is a 5-membered heteroaryl having 1 or 2 heteroatoms independently selected from N, O, and S. In some embodiments, Ar₁ is an unsubstituted 5-membered heteroaryl having 1 or 2 heteroatoms independently selected from N, O, and S. In some embodiments, Ar₁ is unsubstituted thiazolyl. In some embodiments, Ar₁ is unsubstituted isoxazolyl. In some embodiments, Ar₁ is unsubstituted pyrazolyl. In some embodiments, Ar₁ is phenyl substituted with at least one group. In some embodiments, Ar₁ is phenyl substituted with phenoxy. In some embodiments, Ar₁ is phenyl substituted with one phenoxy group.

In some embodiments, Ar₂ is an aryl or a heteroaryl. In some embodiments, Ar₂ is a heteroaryl. In some embodiments, Ar₂ is a 5-14 member heteroaryl. In some embodiments, Ar₂ is a 5-6 member heteroaryl. In some embodiments, Ar₂ is a 6-member heteroaryl. In some embodiments, Ar₂ is a 6-member heteroaryl having one heteroatom selected from N, O, and S. In some embodiments, Ar₂ is pyridinyl. In some embodiments, Ar₂ is unsubstituted pyridinyl.

In some embodiments, Ar₂ is aryl. In some embodiments, Ar₂ is (C₅-C₁₄) aryl. In some embodiments, Ar₂ is phenyl. In some embodiments, Ar₂ is unsubstituted phenyl. In some embodiments, Ar₂ is phenyl substituted with 1-3 groups selected from halogen, (C₁-C₄) alkyl, (C₁-C₄) alkoxy, and optionally substituted 5-6 member heteroaryl. In some embodiments, Ar₂ is phenyl substituted with 1-3 halogen selected from F, Cl, Br, and I. In some embodiments, Ar₂ is phenyl substituted with 1-3 Cl. In some embodiments, Ar₂ is phenyl substituted with two Cl. In some embodiments, Ar₂ is phenyl substituted with 1-3 (C₁-C₄) alkyl. In some embodiments, Ar₂ is phenyl substituted with 1-3 methyl. In some embodiments, Ar₂ is phenyl substituted with one methyl. In some embodiments, Ar₂ is phenyl substituted with 1-3 (C₁-C₄) alkoxy. In some embodiments, Ar₂ is phenyl substituted with 1-3 —OMe. In some embodiments, Ar₂ is phenyl substituted with one —OMe. In some embodiments, Ar₂ is phenyl substituted with 1-3 optionally substituted 5-6 member heteroaryl. In some embodiments, Ar₂ is phenyl substituted with 1-3 optionally substituted 5-member heteroaryl. In some embodiments, Ar₂ is phenyl substituted with one optionally substituted 5-member heteroaryl. In some embodiments, Ar₂ is phenyl substituted with one optionally substituted pyrazolyl. In some embodiments, Ar₂ is phenyl substituted with one unsubstituted pyrazolyl. In some embodiments, Ar₂ is phenyl substituted with one pyrazolyl substituted with 1-3 (C₁-C₄) alkyl. In some embodiments, Ar₂ is phenyl substituted with one pyrazolyl substituted with one methyl.

In some embodiments, certain optional groups that are substitutents on the compound of Formula (I) are referred to as R₁₀, R₁₁, and R₁₂. When a compound of Formula (I) is substituted with more than one R₁₀, or more than one R₁₁, or more than one R₁₂, the groups can be the same or different. For instance, a compound can be substituted with two R₁₀ which are both chlorides, or one R₁₀ which is chloride and one which is methyl.

In some embodiments, the compound of Formula (I) does not include a bicyclic aryl. In some embodiments, the compound of Formula (I) does not include a bicyclic heteroaryl.

In some embodiments, the compound of Formula (I) is a compound of Formula (VIII), the compound is a compound of formula (VIII):

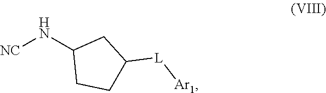

wherein: L is —C(O)NR₄— or —NR₄C(O)—;

when L is —NR₄C(O)—, each R₁₀ is independently halogen, (C₁-C₆) alkyl, (C₃-C₆) cycloalkyl, (C₁-C₆) alkoxy, aryloxy, or aryl or heteroaryl optionally substituted with one or more R₁₁, each R₁₁ being independently selected from hydroxyl, halogen, or cyano; and

when L is —C(O)NR₄—, each R₁₀ is independently halogen, (C₁-C₆) alkoxy, aryloxy, or aryl or heteroaryl optionally substituted with one or more R₁₁, each R₁₁ being independently selected from hydroxyl, halogen, or cyano;

R₄ is hydrogen or (C₁-C₆) alkyl; and

Ar₁ is an aryl or heteroaryl optionally substituted with one or more R₁₀.

In some embodiments, the compound of Formula (I) is a compound of Formula (IX), wherein the substituents R₄ and Ar₁ are as defined above:

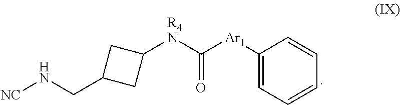

In some embodiments, the compound of any one of Formulas (I)-(IX) do not have any of the following structures:

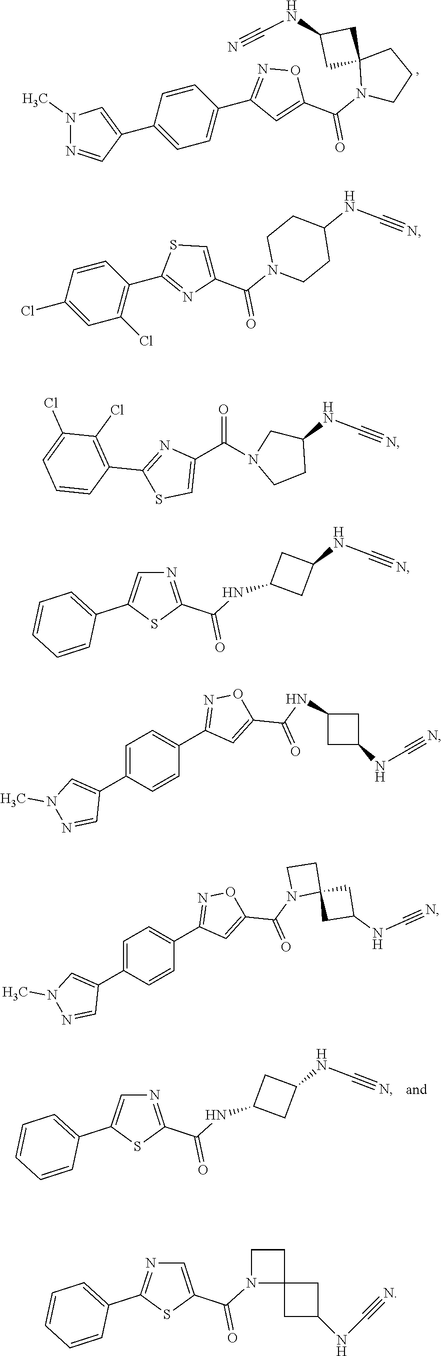

### Methods of Preparing the Compounds

The compounds of the present disclosure may be made by a variety of methods, including standard chemistry. Suitable synthetic routes are depicted in the examples given below.

The compounds of the present disclosure, i.e., compounds of formulas (I)-(VII) or a pharmaceutically acceptable salt thereof, may be prepared by methods known in the art of organic synthesis as set forth in part by the synthetic schemes depicted in the examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula (I)-(VII).

Those skilled in the art will recognize stereocenters may exist in the compounds of Formula (I)-(VII). Accordingly, the present disclosure includes both possible stereoisomers (unless otherwise indicated and/or specified in the synthesis) and includes not only racemic compounds but the individual enantiomers and/or diastereomers as well. Unless otherwise indicated, when a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

### Methods of Using the Disclosed Compounds

One aspect of the present disclosure relates to a compound of Formula (I)-(VII) for use in medicine. Another aspect of the present disclosure relates to a method of modulating TRABID, comprising administering to a patient in need thereof a therapeutically effective amount of a compound of any one of formulas (I)-(VII). Another aspect of the present disclosure relates to a method of inhibiting one or more of TRABID, comprising administering to a patient in need thereof a therapeutically effective amount of a compound of any one of formulas (I)-(VII). In another aspect, the present disclosure relates to a method of inhibiting TRABID, comprising administering to a patient in need thereof a therapeutically effective amount of a pharmaceutical composition comprising a compound of any one of formulas (I)-(VII).

TRABID Inhibitor Compounds are useful in the development of pharmaceutical compositions suitable for treatment of autoimmune inflammatory diseases including, but not limited to, psoriasis. TRABID Inhibitor Compounds are useful for treating disease states that are responsive to the inhibition of TRABID. This disclosure relates to the treatment of psoriasis, including the treatment of patients diagnosed with psoriasis by the administration of a compound that inhibits TRABID.

### EXAMPLES

### Materials and Instrumentation

All solvents used were commercially available and were used without further purification. Reactions were typically run using anhydrous solvents under an inert atmosphere of nitrogen.

Proton NMR spectra was recorded using a Bruker Plus 400 NMR Spectrometer; The deuterated solvent (CD₃OD) contained typically 0.03% to 0.05% v/v tetramethylsilane, which was used as the reference signal (set at d 0.00 for ¹H).

LCMS analyses were performed on a SHIMADZU LCMS consisting of an UFLC 20-AD and LCMS 2020 MS detector. The column was used was a Shim-pack XR-ODS, 2.2 μm, 3.0×50 mm. The instrument using reverse-phase conditions (acetonitrile/water, containing 0.05% acetic acid).

Preparative HPLC using a ‘neutral method’ and mass-triggered fraction collection was completed using a 6 minute method on an instrument equipped with a Waters 2545 Binary Gradient Module, Waters 3100/ZQ Mass Detector, and Waters UV/998 PDA detector. Mobile phase A was water and mobile phase B was acetonitrile. The mobile phase gradient used for this method was as follows:

1) Hold 35% B for 0.9 minutes;

2) 35 to 45% B in 0.01 minutes;

3) 45 to 85% B in 3.84 minutes;

4) 85 to 100% B in 0.01 minutes;

5) Hole 100% B for 0.74 minutes.

A Waters SunFire C18 OBD Prep Column, 100 Å, 5 μm, 19 mm×50 mm column at ambient temperature was used for purification. A flow rate of 23 mL per minute from the binary pump and 2 mL per minute acetonitrile at column dilution was used. The UV detector was set to monitor wavelengths of 220 nm and 254 nm. The mass spectrometer was set to detect in positive mode and used electrospray ionization for ionization of the analyte.

### The Following Abbreviations are Used in the Examples Below and Elsewhere Herein:

| δ | chemical shift |
|---|---|
| ACN | Acetonitrile |
| DCM | Dichloromethane or methylene chloride |
| DIEA | N,N-Diisopropylethylamine |
| DMSO | Dimethyl sulfoxide |
| DMF | N,N-Dimethylformamide |
| EtOAc | Ethyl acetate |
| EtOH | Ethanol |
| FA | Formic Acid |
| h | hour |
| ¹H NMR | proton nuclear magnetic resonance |
| HATU | 2-(3H-[1,2,3]Triazolo[4,5-b]pyridin-3-yl)-1,1,3,3- |
| | tetramethylisouronium hexafluorophosphate |
| HCl | Hydrochloric acid |
| HPLC | high performance liquid chromatography |
| Hz | Hertz |
| LCMS | liquid chromatography/mass spectrometry |
| min | minutes |
| MS | mass spectrometry |
| o/n | Overnight |
| Pd(dppf)Cl₂ | [1,1′-Bis(diphenylphosphino)ferrocene] |
| | dichloropalladium(II) |
| Py | pyridine |
| Rt | Retention time |
| Rt | Room temperature |
| TFA | Trifluoroacetic acid |

### Example 1

### Synthesis of cyano([[(3S)-1-[2-(2-phenoxyphenyl)-1,3-thiazole-4-carbonyl]pyrrolidin-3-yl]methyl])amine (1)

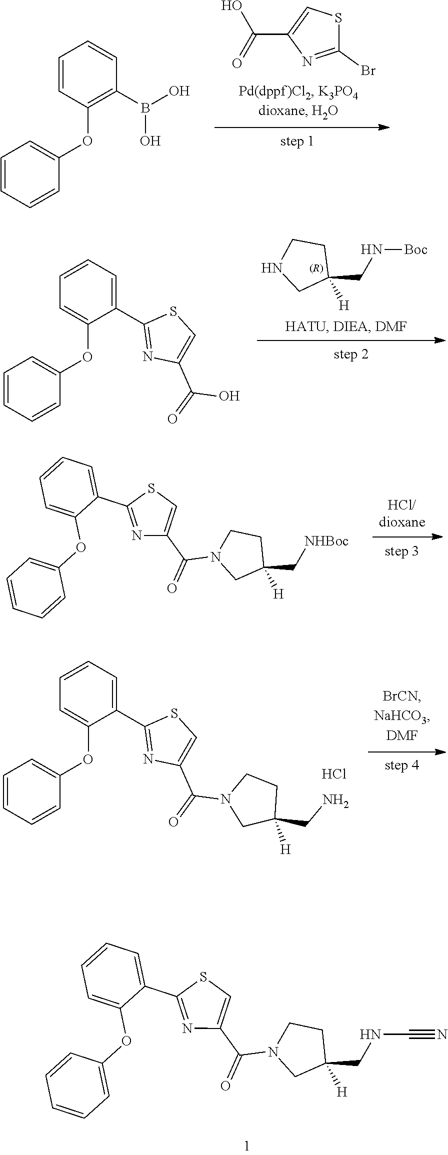

### Step 1. 2-(2-phenoxyphenyl)-1,3-thiazole-4-carboxylic acid

A mixture of 2-phenoxyphenylboronic acid (3.00 g, 14.0 mmol), 2-bromo-1,3-thiazole-4-carboxylic acid (4.65 g, 22.4 mmol), Pd(dppf)Cl₂ (2.10 g, 2.87 mmol) and K₃PO₄ (9.22 g, 43.4 mmol) in dioxane (60 mL) and H₂O (30 mL) was stirred for 3 hours at 90° C. under a nitrogen atmosphere. After cooling to room temperature, the reaction mixture was poured into water/ice (100 mL) and extracted with CH₂C₂ (3×100 mL). The aqueous layer was concentrated under reduced pressure.

The residue was purified by reverse flash chromatography with the following conditions: column, C₁₈ silica gel, 330 g, 20-35 μm; mobile phase, water with FA (0.1%) and ACN (0% to 100% gradient in 25 minutes); detector, UV 254/220 nm. The collected fraction was concentrated under reduced pressure to afford 2-(2-phenoxyphenyl)-1,3-thiazole-4-carboxylic acid (950 mg, 22%) as a light brown solid. LCMS (ES, m/z)⁺: 298 [M+H]⁺.

### Step 2. tert-butyl N-[[(3S)-1-[2-(2-phenoxyphenyl)-1,3-thiazole-4-carbonyl]pyrrolidin-3-yl]methyl]carbamate

To a stirred mixture of 2-(2-phenoxyphenyl)-1,3-thiazole-4-carboxylic acid (300 mg, 1.01 mmol) and HATU (576 mg, 1.52 mmol) in DMF (6 mL) were added DIEA (500 uL, 3.03 mmol) and tert-butyl N-[(3R)-pyrrolidin-3-ylmethyl]carbamate (263 mg, 1.31 mmol) at 0° C. The resulting mixture was stirred for 2 hours at 25° C. under nitrogen atmosphere.

The mixture was purified by reverse flash chromatography with the following conditions: column, C₁₈ silica gel, 120 g, 20-35 μm; mobile phase, water with NH₄HCO₃ (10 mmol/L) and ACN (0% to 70% gradient in 20 minutes); detector, UV 254/220 nm. The collected fraction was concentrated under reduced pressure to afford tert-butyl N-[[(3S)-1-[2-(2-phenoxyphenyl)-1,3-thiazole-4-carbonyl]pyrrolidin-3-yl]methyl]carbamate (420 mg, 82%) as a light brown solid. LCMS (ES, m/z)+: 480 [M+H]⁺.

### Step 3. 1-[(3S)-1-[2-(2-phenoxyphenyl)-1,3-thiazole-4-carbonyl]pyrrolidin-3-yl]methanamine hydrochloride

To a stirred mixture of tert-butyl N-[[(3S)-1-[2-(2-phenoxyphenyl)-1,3-thiazole-4-carbonyl]pyrrolidin-3-yl]methyl]carbamate (300 mg, 0.63 mmol) in DCM (20 mL) was added HCl in 1,4-dioxane (10 mL, 4M) dropwise at 0° C. The resulting mixture was stirred for 2 hours at 25° C. The resulting mixture was concentrated under reduced pressure to afford 1-[(3S)-1-[2-(2-phenoxyphenyl)-1,3-thiazole-4-carbonyl]pyrrolidin-3-yl]methanamine hydrochloride (260 mg, 99%) as a brown oil. LCMS (ES, m/z)⁺: 380 [M−HCl+H]⁺.

### Step 4. cyano([[(3S)-1-[2-(2-phenoxyphenyl)-1,3-thiazole-4-carbonyl]pyrrolidin-3-yl]methyl])amine (1)

To a stirred mixture of 1-[(3S)-1-[2-(2-phenoxyphenyl)-1,3-thiazole-4-carbonyl]pyrrolidin-3-yl]methanamine hydrochloride (100 mg, 0.24 mmol) in DMF (3 mL) was added NaHCO₃ (100 mg, 1.19 mmol) and BrCN (25 mg, 0.24 mmol) dropwise at 0° C. The resulting mixture was stirred for 2 hours at 25° C.

The mixture was purified by Prep-HPLC with the following conditions: Column, XBridge Shield RP18 OBD Column, 5 μm, 19×150 mm; mobile phase, water and ACN (45% PhaseB up to 65% in 7 minutes); Detector, UV254/220 nm. The collected fraction was lyophilized to afford cyano([[(3S)-1-[2-(2-phenoxyphenyl)-1,3-thiazole-4-carbonyl]pyrrolidin-3-yl]methyl]) amine (1) (38.7 mg, 38%) as a white solid.

1 ¹H NMR (400 MHz, DMSO-d6) δ (ppm): 8.43-8.38 (m, 1H), 8.26 (d, J=4.8 Hz, 1H), 7.53-7.43 (m, 3H), 7.38-7.32 (m, 1H), 7.23-7.20 (m, 1H), 7.13-7.06 (m, 3H), 6.94-6.91 (m, 1H), 4.15-4.06 (m, 1H), 3.76-3.65 (m, 2H), 3.30-3.25 (m, 1H), 3.07-3.02 (m, 2H), 2.51-2.41 (m, 1H), 2.14-1.98 (m, 1H), 1.69-1.67 (m, 1H). LCMS (ES, m/z)+: 405 [M+H]⁺.

### Example 2

### Synthesis of cyano([[(3R)-1-[2-(2-phenoxyphenyl)-1,3-thiazole-4-carbonyl]pyrrolidin-3-yl]methyl])amine (2)

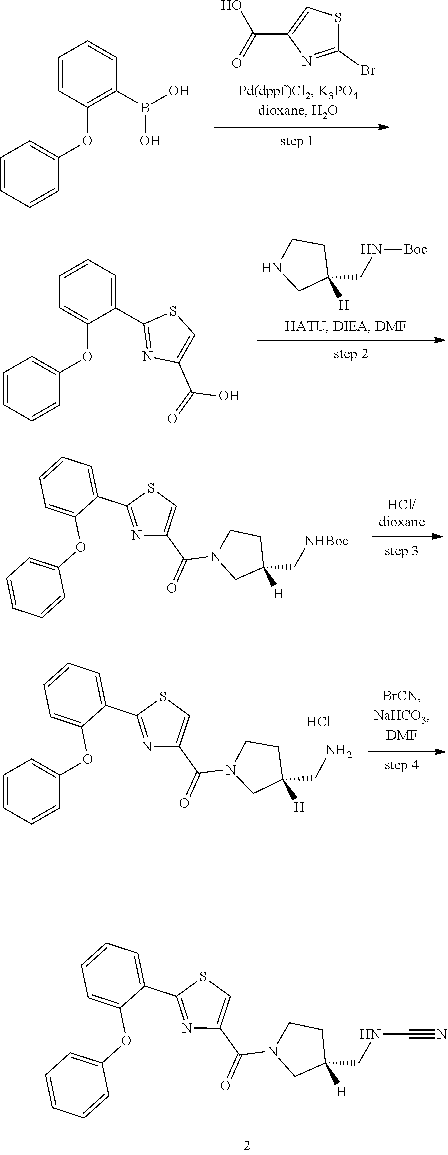

### Step 1. 2-(2-phenoxyphenyl)-1,3-thiazole-4-carboxylic acid

A mixture of 2-phenoxyphenylboronic acid (3.00 g, 14.0 mmol), 2-bromo-1,3-thiazole-4-carboxylic acid (4.65 g, 22.4 mmol), Pd(dppf)Cl₂ (2.10 g, 2.87 mmol) and K₃PO₄ (9.22 g, 43.4 mmol) in dioxane (60 mL) and H₂O (30 mL) was stirred for 3 hours at 90° C. under a nitrogen atmosphere. After cooling to room temperature, the reaction mixture was poured into water/ice (100 mL) and extracted with CH₂C₂ (3×100 mL). The aqueous layer was concentrated under reduced pressure.

The residue was purified by reverse flash chromatography with the following conditions: column, C18 silica gel, 330 g, 20-35 μm; mobile phase, water with FA (0.1%) and ACN (0% to 100% gradient in 25 minutes); detector, UV 254/220 nm. The collected fraction was concentrated under reduced pressure to afford 2-(2-phenoxyphenyl)-1,3-thiazole-4-carboxylic acid (950 mg, 22%) as a light brown solid. LCMS (ES, m/z)⁺: 298 [M+H]⁺.

### Step 2. tert-butyl N-[[(3R)-1-[2-(2-phenoxyphenyl)-1,3-thiazole-4-carbonyl]pyrrolidin-3-yl]methyl]carbamate

To a stirred mixture of 2-(2-phenoxyphenyl)-1,3-thiazole-4-carboxylic acid (300 mg, 1.00 mmol) and HATU (576 mg, 1.51 mmol) in DMF (6 mL) was added DIEA (500 uL, 3.03 mmol) and tert-butyl N-[(3S)-pyrrolidin-3-ylmethyl]carbamate (222 mg, 1.11 mmol) at 0° C. The resulting mixture was stirred for 2 hours at 25° C.

The mixture was purified by reverse flash chromatography with the following conditions: column, C18 silica gel, 120 g, 20-35 μm; mobile phase, water with NH₄HCO₃ (10 mmol/L) and ACN (0% to 70% gradient in 20 minutes); detector, UV 254/220 nm. The collected fraction was concentrated under reduced pressure to afford tert-butyl N-[[(3R)-1-[2-(2-phenoxyphenyl)-1,3-thiazole-4-carbonyl]pyrrolidin-3-yl]methyl]carbamate (474 mg, 98%) as a light brown solid. LCMS (ES, m/z)⁺: 480 [M+H]⁺.

### Step 3. 1-[(3R)-1-[2-(2-phenoxyphenyl)-1,3-thiazole-4-carbonyl]pyrrolidin-3-yl]methanamine hydrochloride

To a stirred mixture of tert-butyl N-[[(3R)-1-[2-(2-phenoxyphenyl)-1,3-thiazole-4-carbonyl]pyrrolidin-3-yl]methyl]carbamate (300 mg, 0.63 mmol) in DCM (20 mL) was added HCl in 1,4-dioxane (15 mL, 4M) dropwise at 0° C. The resulting mixture was stirred for 1 hour at 25° C. under nitrogen atmosphere. The resulting mixture was concentrated under reduced pressure to afford 1-[(3R)-1-[2-(2-phenoxyphenyl)-1,3-thiazole-4-carbonyl]pyrrolidin-3-yl]methanamine hydrochloride (250 mg, 96%) as a brown oil. LCMS (ES, m/z)⁺: 380 [M−HCl+H]⁺.

### Step 4. cyano([[(3R)-1-[2-(2-phenoxyphenyl)-1,3-thiazole-4-carbonyl]pyrrolidin-3-yl]methyl])amine (2)

To a stirred mixture of 1-[(3R)-1-[2-(2-phenoxyphenyl)-1,3-thiazole-4-carbonyl]pyrrolidin-3-yl]methanamine hydrochloride (100 mg, 0.24 mmol) in DMF (3 mL) was added NaHCO₃ (100 mg, 1.19 mmol) and BrCN (25 mg, 0.24 mmol) dropwise at 0° C. The resulting mixture was stirred for 2 hours at 25° C. under nitrogen atmosphere.

The mixture was purified by Prep-HPLC with the following conditions: Column, XBridge Shield RP18 OBD Column, 5 μm, 19×150 mm; mobile phase, water and ACN (45% PhaseB up to 65% in 7 minutes); Detector, UV254/220 nm. The collected fraction was lyophilized to afford cyano([[(3R)-1-[2-(2-phenoxyphenyl)-1,3-thiazole-4-carbonyl]pyrrolidin-3-yl]methyl])amine (2) (25.6 mg, 25%) as a white solid.

2 ¹H NMR (400 MHz, DMSO-d6) δ (ppm): 8.45-8.37 (m, 1H), 8.26 (d, J=5.2 Hz, 1H), 7.53-7.43 (m, 3H), 7.38-7.32 (m, 1H), 7.23-7.20 (m, 1H), 7.13-7.06 (m, 3H), 6.94-6.91 (m, 1H), 4.15-4.06 (m, 1H), 3.74-3.53 (m, 2H), 3.32-3.25 (m, 1H), 3.07-3.02 (m, 2H), 2.50-2.40 (m, 1H), 2.15-1.98 (m, 1H), 1.79-1.60 (m, 1H). LCMS (ES, m/z)⁺: 405 [M+H]⁺.

### Example 3

### Synthesis of 4-(cyanoamino)-N-[2-(3-methoxyphenyl)-1,3-thiazol-5-yl]butanamide (3)

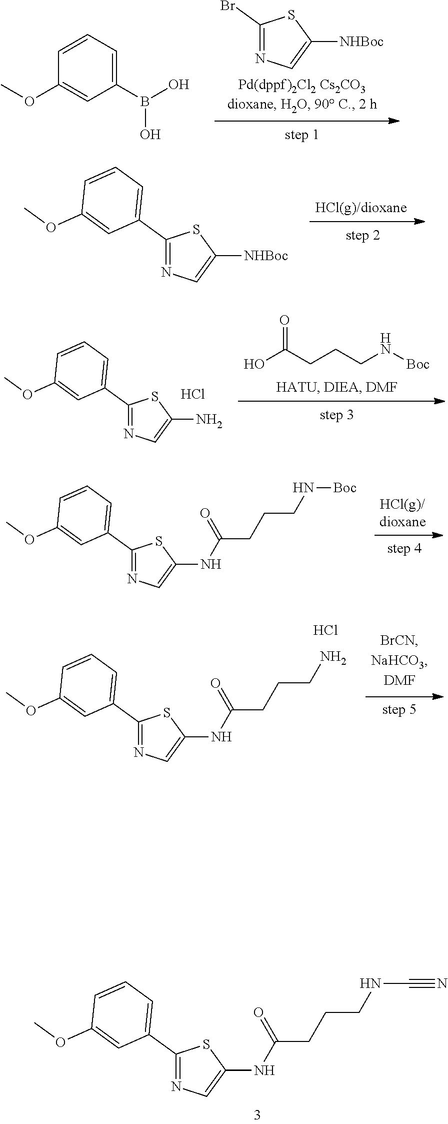

### Step 1. tert-butyl N-[2-(3-methoxyphenyl)-1,3-thiazol-5-yl]carbamate

To a stirred solution of tert-butyl N-(2-bromo-1,3-thiazol-5-yl)carbamate (6.00 g, 21.0 mmol), 3-methoxyphenylboronic acid (4.80 g, 31.5 mmol) and Pd(dppf)Cl2 (1.03 g, 1.40 mmol) in dioxane (60 mL) was added K3PO4 (13.4 g, 63.1 mmol) in H2O (20 mL) under a nitrogen atmosphere. The resulting mixture was stirred for 1 hour at 90° C. under a nitrogen atmosphere. The mixture was cooled to room temperature. The resulting mixture was diluted with water (500 mL) and extracted with EtOAc (3×500 mL). The combined organic layers were dried over anhydrous Na₂SO₄ and concentrated under reduced pressure. The residue was purified by silica gel chromatography (eluting with 1:1 ethyl acetate/petroleum ether) to afford tert-butyl N-[2-(3-methoxyphenyl)-1,3-thiazol-5-yl]carbamate as a yellow solid (4.80 g, 71%). LCMS (ES, m/z)⁺: 307 [M+H]⁺.

### Step 2. 2-(3-methoxyphenyl)-1,3-thiazol-5-amine hydrochloride

To a stirred solution of tert-butyl N-[2-(3-methoxyphenyl)-1,3-thiazol-5-yl]carbamate (3.80 g, 11.9 mmol) in MeOH (10 mL) was added HCl(gas) in 1,4-dioxane (20 mL). The resulting mixture was stirred for 15 hours at 24° C. The mixture was concentrated under vacuum to afford 2-(3-methoxyphenyl)-1,3-thiazol-5-amine hydrochloride as a yellow solid (2.80 g, 93%). LCMS (ES, m/z)⁺: 207 [M−HCl+H]⁺.

### Step 3. tert-butyl N-(3-[[2-(3-methoxyphenyl)-1,3-thiazol-5-yl]carbamoyl]propyl) carbamate

To a stirred solution of 4-[(tert-butoxycarbonyl)amino]butanoic acid (120 mg, 0.59 mmol) and HATU (225 mg, 0.59 mmol) in DMF (3 mL) was added 2-(3-methoxyphenyl)-1,3-thiazol-5-amine hydrochloride (100 mg, 0.39 mmol) and DIEA (195 uL, 1.18 mmol). The resulting mixture was stirred for 5 hours at 24° C. The resulting mixture was diluted with water (50 mL) and extracted with EtOAc (3×50 mL). The combined organic layers were dried over anhydrous Na₂SO₄ and concentrated under reduced pressure. The residue was purified by silica gel column chromatography, eluted with petroleum ether/EtOAc (1:1), to afford tert-butyl N-(3-[[2-(3-methoxyphenyl)-1,3-thiazol-5-yl]carbamoyl]propyl)carbamate (100 mg, 62%) as a yellow solid. LCMS (ES, m/z)⁺: 392 [M+H]⁺.

### Step 4. 4-amino-N-[2-(3-methoxyphenyl)-1,3-thiazol-5-yl]butanamide hydrochloride

To a stirred solution of tert-butyl N-(3-[[2-(3-methoxyphenyl)-1,3-thiazol-5-yl]carbamoyl]propyl)carbamate (90 mg, 0.22 mmol) in MeOH (1 mL) was added HCl (gas) in 1,4-dioxane (2 mL). The resulting mixture was stirred for 2 hours at 24° C. The mixture was concentrated under vacuum to afford 4-amino-N-[2-(3-methoxyphenyl)-1,3-thiazol-5-yl]butanamide hydrochloride (70 mg, 92%) as a yellow solid. LCMS (ES, m/z)⁺: 292 [M−HCl+H]⁺.

### Step 5. 4-(cyanoamino)-N-[2-(3-methoxyphenyl)-1,3-thiazol-5-yl]butanamide (3)

To a stirred mixture of 4-amino-N-[2-(3-methoxyphenyl)-1,3-thiazol-5-yl]butanamide hydrochloride (50 mg, 0.14 mmol) and NaHCO₃ (61 mg, 0.73 mmol) in DMF (1.5 mL) was added a solution of BrCN (15 mg, 0.14 mmol) in DMF (0.5 mL) dropwise at 0° C. The resulting mixture was stirred for 2 hours at 24° C. The mixture was diluted with water (30 mL) and extracted with EtOAc (3×30 mL). The combined organic layers were dried over anhydrous Na₂SO₄ and concentrated under reduced pressure.

The crude product was purified by prep-HPLC with the following conditions (Column: XBridge Shield RP18 OBD Column, 5 μm, 19×150 mm; Mobile Phase A: water, Mobile Phase B: ACN; Flow rate: 25 mL/minute; Gradient: 40 B to 60 B in 8 minutes; UV 220 nm). The product phase was lyophilized to afford 4-(cyanoamino)-N-[2-(3-methoxyphenyl)-1,3-thiazol-5-yl]butanamide (3) (24.7 mg, 45%) as a white solid.

3 ¹H-NMR (DMSO-d6, 400 MHz) δ (ppm): 11.49 (s, 1H), 7.56 (s, 1H), 7.44-7.36 (m, 3H), 7.01-6.98 (m, 1H), 6.81 (br s, 1H), 3.83 (s, 3H), 3.02-2.97 (m, 2H), 2.51-2.46 (m, 2H), 1.85-1.78 (m, 2H). LCMS (ES, m/z)⁺: 317 [M+H]⁺.

### Example 4

### Synthesis of (2R)-4-(cyanoamino)-N-[2-(3-methoxyphenyl)-1,3-thiazol-5-yl]-2-methylbutanamide (4) and (2S)-4-(cyanoamino)-N-[2-(3-methoxyphenyl)-1,3-thiazol-5-yl]-2-methylbutanamide (6)

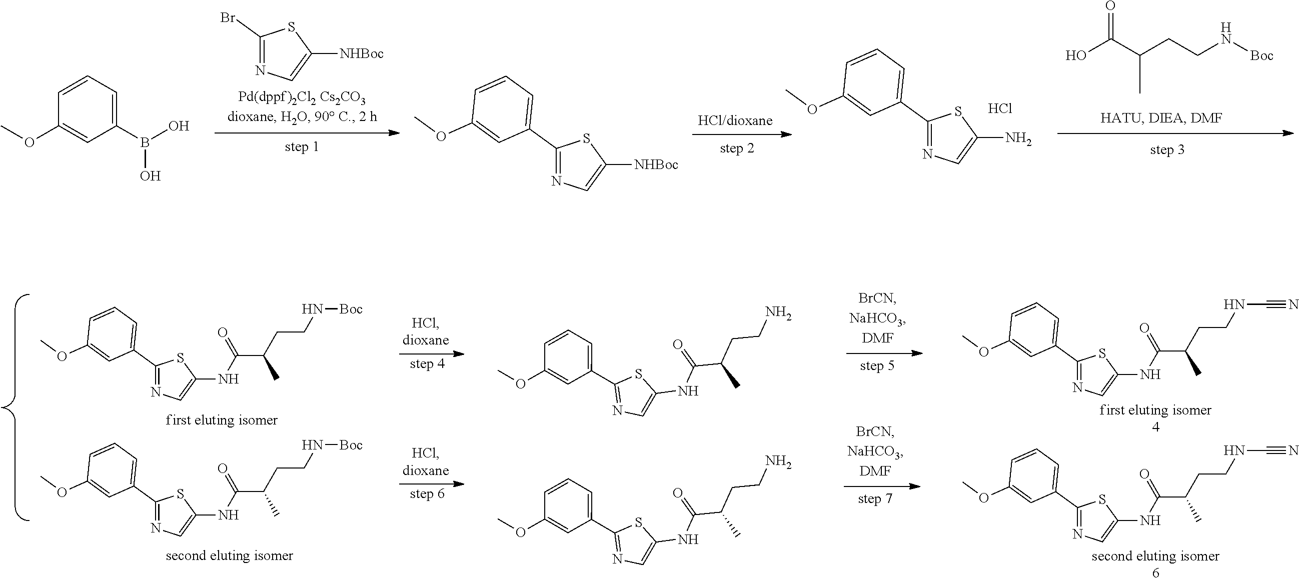

### Step 1. tert-butyl N-[2-(3-methoxyphenyl)-1,3-thiazol-5-yl]carbamate

To a stirred solution of tert-butyl N-(2-bromo-1,3-thiazol-5-yl)carbamate (6.00 g, 21.0 mmol), 3-methoxyphenylboronic acid (4.80 g, 31.5 mmol) and Pd(dppf)Cl₂ (1.03 g, 1.40 mmol) in dioxane (60 mL) was added K₃PO₄ (13.4 g, 63.1 mmol) in H₂O (20 mL) under a nitrogen atmosphere. The resulting mixture was stirred for 1 hour at 90° C. under a nitrogen atmosphere. The mixture was cooled to room temperature. The resulting mixture was diluted with water (500 mL) and extracted with EtOAc (3×500 mL). The combined organic layers were dried over anhydrous Na₂SO₄ and concentrated under reduced pressure. The residue was purified by silica gel chromatography (eluting with 1:1 ethyl acetate/petroleum ether) to afford tert-butyl N-[2-(3-methoxyphenyl)-1,3-thiazol-5-yl]carbamate as a yellow solid (4.80 g, 71%). LCMS (ES, m/z)⁺: 307 [M+H]⁺.

### Step 2. 2-(3-methoxyphenyl)-1,3-thiazol-5-amine hydrochloride

To a stirred solution of tert-butyl N-[2-(3-methoxyphenyl)-1,3-thiazol-5-yl]carbamate (3.80 g, 11.9 mmol) in MeOH (10 mL) was added HCl in 1,4-dioxane (20 mL). The resulting mixture was stirred for 15 hours at 24° C. The mixture was concentrated under vacuum to afford 2-(3-methoxyphenyl)-1,3-thiazol-5-amine hydrochloride as a yellow solid (2.80 g, 93%). LCMS (ES, m/z)⁺: 207 [M−HCl+H]⁺.

### Step. 3 tert-butyl N-[(3R)-3-{[2-(3-methoxyphenyl)-1,3-thiazol-5-yl]carbamoyl}-3-methylpropyl]carbamate and tert-butyl N-[(3S)-3-{[2-(3-methoxyphenyl)-1,3-thiazol-5-yl]carbamoyl}-3-methylpropyl]carbamate

To a stirred mixture of 4-[(tert-butoxycarbonyl)amino]-2-methylbutanoic acid (240 mg, 1.11 mmol) and HATU (525 mg, 1.38 mmol) in DMF (5 mL) was added 2-(3-methoxyphenyl)-1,3-thiazol-5-amine hydrochloride (200 mg, 0.83 mmol) and DIEA (456 μL, 2.76 mmol) at 0° C. The resulting mixture was stirred for 12 hours at 25° C. The mixture was poured into water (20 mL) and extracted with EtOAc (3×20 mL). The combined organic layers were washed with brine (50 mL), dried over anhydrous Na₂SO₄ and concentrated under reduced pressure.

The residue was purified by Prep-TLC (eluted with hexane/EtOAc (1:1)) to afford racemic product (120 mg) as a white solid. The racemate was separated by chiral HPLC (Column: CHIRALPAK IG, 20×250 mm, 5 μm; Mobile Phase A: Hex, Mobile Phase B: EtOH; Flow rate: 20 mL/minutes; Gradient: 30 B to 30 B in 15 minutes; UV 220/254 nm; Rt1: 9.241 minutes; Rt2: 10.898 minutes; Injection Volume: 0.3 ml; Number Of Runs: 12) to afford tert-butyl N-[(3R)-3-{[2-(3-methoxyphenyl)-1,3-thiazol-5-yl]carbamoyl}-3-methylpropyl]carbamate (first eluting isomer, Rt1: 9.241) (50 mg, 30%) and tert-butyl N-[(3S)-3-{[2-(3-methoxyphenyl)-1,3-thiazol-5-yl]carbamoyl}-3-methylpropyl]carbamate (second eluting isomer, RT2: 10.898) (50 mg, 30%) as off-white solids. First eluting isomer LCMS (ES, m/z)⁺: 406 [M+H]⁺. Second eluting isomer LCMS (ES, m/z)⁺: 406 [M+H]⁺.

### Step. 4 (2R)-4-amino-N-[2-(3-methoxyphenyl)-1,3-thiazol-5-yl]-2-methylbutanamide hydrochloride

A mixture of tert-butyl N-[(3R)-3-[[2-(3-methoxyphenyl)-1,3-thiazol-5-yl]carbamoyl]-3-methylpropyl]carbamate (50 mg, 0.12 mmol) in HCl (4 M) in dioxane (2 mL) was stirred for 1 hour at 25° C. The resulting mixture was concentrated under reduced pressure to afford (2R)-4-amino-N-[2-(3-methoxyphenyl)-1,3-thiazol-5-yl]-2-methylbutanamide hydrochloride (30 mg, 71%) as a white solid. LCMS (ES, m/z)⁺: 306 [M−HCl+H]⁺.

### Step 5. (2R)-4-(cyanoamino)-N-[2-(3-methoxyphenyl)-1,3-thiazol-5-yl]-2-methylbutanamide (4)

To a stirred mixture of (2R)-4-amino-N-[2-(3-methoxyphenyl)-1,3-thiazol-5-yl]-2-methylbutanamide hydrochloride (30 mg, 0.09 mmol) and NaHCO₃ (16 mg, 0.19 mmol) in DMF (1 mL) was added BrCN (10 mg, 0.09 mmol) at 0° C. The resulting mixture was stirred for 1 hour at 25° C.

The mixture was purified by Prep-HPLC with the following conditions: Column, XBridge Shield RP18 OBD Column, 5 μm, 19×150 mm; mobile phase, water with 10 mM NH₄HCO₃ and ACN (30% up to 50% in 7 minutes); Detector, UV 254/220 nm. The collected fraction was lyophilized to afford (2R)-4-(cyanoamino)-N-[2-(3-methoxyphenyl)-1,3-thiazol-5-yl]-2-methylbutanamide (4) (19 mg, 65%) as a white solid.

4 ¹H-NMR (DMSO, ppm): 11.48 (s, 1H), 7.59 (s, 1H), 7.43-7.36 (m, 3H), 7.00-6.98 (m, 1H), 6.79-6.77 (m, 1H), 3.82 (s, 3H), 2.95-2.90 (m, 2H), 2.67-2.60 (m, 1H), 1.90-1.81 (m, 1H), 1.65-1.62 (m, 1H), 1.17 (d, J=6.8 Hz, 3H). LCMS (ES, m/z)⁺: 331 [M+H]⁺.

### Step 6. (2S)-4-amino-N-[2-(3-methoxyphenyl)-1,3-thiazol-5-yl]-2-methylbutanamide hydrochloride

A mixture of tert-butyl N-[(3S)-3-[[2-(3-methoxyphenyl)-1,3-thiazol-5-yl]carbamoyl]-3-methylpropyl]carbamate (50 mg, 0.12 mmol) in HCl (4 M) in dioxane (2 mL) was stirred for 1 hour at 25° C. The resulting mixture was concentrated under reduced pressure to afford (2S)-4-amino-N-[2-(3-methoxyphenyl)-1,3-thiazol-5-yl]-2-methylbutanamide hydrochloride (30 mg, 71%) as a white solid. LCMS (ES, m/z)⁺: 306 [M−HCl+H]⁺.

### Step 7. (2S)-4-(cyanoamino)-N-[2-(3-methoxyphenyl)-1,3-thiazol-5-yl]-2-methylbutanamide (6)

To a stirred mixture of (2S)-4-amino-N-[2-(3-methoxyphenyl)-1,3-thiazol-5-yl]-2-methylbutanamide hydrochloride (30 mg, 0.09 mmol) and NaHCO₃ (16 mg, 0.19 mmol) in DMF (1 mL) was added BrCN (10 mg, 0.09 mmol) at 0° C. The resulting mixture was stirred for 1 hour at 25° C.

The mixture was purified by Prep-HPLC with the following conditions: Column, XBridge Shield RP18 OBD Column, 5 μm, 19×150 mm; mobile phase, water with 10 mM NH₄HCO₃ and ACN (30% up to 50% in 7 minutes); Detector, UV 254/220 nm. The collected fraction was lyophilized to afford (2S)-4-(cyanoamino)-N-[2-(3-methoxyphenyl)-1,3-thiazol-5-yl]-2-methylbutanamide (6) (24 mg, 83%) as a white solid.

6 ¹H-NMR (DMSO, ppm): 11.48 (s, 1H), 7.59 (s, 1H), 7.43-7.36 (m, 3H), 7.00-6.98 (m, 1H), 6.79-6.77 (m, 1H), 3.82 (s, 3H), 2.95-2.90 (m, 2H), 2.67-2.60 (m, 1H), 1.90-1.81 (m, 1H), 1.65-1.62 (m, 1H), 1.17 (d, J=6.8 Hz, 3H). LCMS (ES, m/z)⁺: 331 [M+H]⁺.

### Example 5

### Synthesis of N-[3-(cyanoamino)propyl]-2-(3-methoxyphenyl)-1,3-thiazole-5-carboxamide (5)

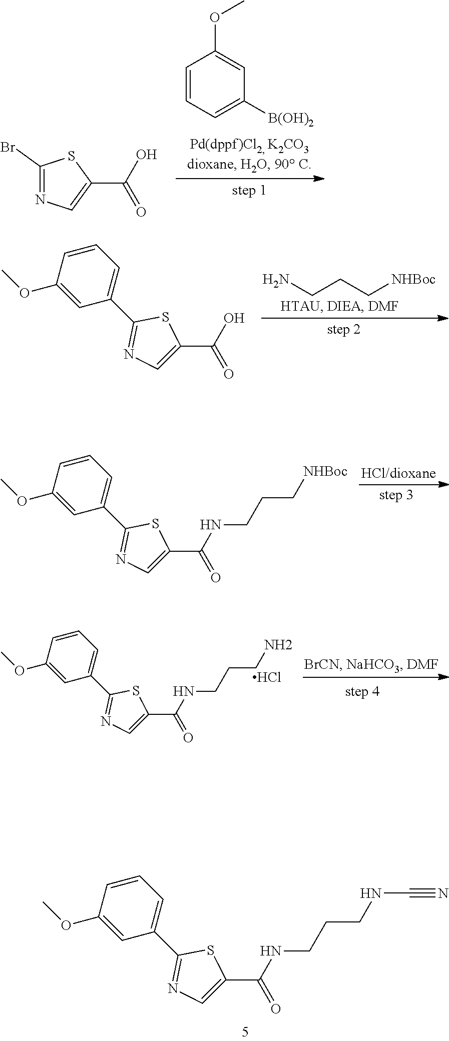

### Step 1. 2-(3-methoxyphenyl)-1,3-thiazole-5-carboxylic acid

To a stirred solution of 2-bromo-1,3-thiazole-5-carboxylic acid (1.00 g, 4.71 mmol), 3-methoxyphenylboronic acid (1.07 g, 7.06 mmol) and Pd(dppf)Cl₂ (689 mg, 0.94 mmol) in dioxane (30 mL) was added Cs₂CO₃ (4.60 g, 14.1 mmol) in H₂O (10 mL). The resulting mixture was stirred for 10 hours at 90° C. under nitrogen atmosphere. The mixture was cooled to room temperature and concentrated under reduced pressure. The resulting mixture was acidified to pH 5 with HCl (2M) and extracted with CH₂Cl₂ (3×200 mL). The combined organic layers were dried over anhydrous Na₂SO₄ and concentrated under reduced pressure.

The crude product was purified by reversed phase column chromatography (Column, C18 silica gel, 80 g, 40-60 μm, 60 Å; mobile phase, water (0.05% TFA) and ACN (0% up to 80% ACN in 30 minutes); Detector, UV 220 & 254 nm) to afford 2-(3-methoxyphenyl)-1,3-thiazole-5-carboxylic acid (500 mg, 43%) as a yellow solid. LCMS (ES, m/z)⁺: 236 [M+H]⁺.

### Step 2. tert-butyl N-(3-[[2-(3-methoxyphenyl)-1,3-thiazol-5-yl]formamido]propyl)carbamate

To a stirred solution of 2-(3-methoxyphenyl)-1,3-thiazole-5-carboxylic acid (300 mg, 1.27 mmol) and tert-butyl N-(3-aminopropyl)carbamate (222 mg, 1.27 mmol) in DMF (12 mL) were added HATU (728 mg, 1.9 mmol) and DIEA (840 uL, 5.09 mmol). The resulting mixture was stirred for 3 hours at 25° C. The resulting mixture was poured into water (50 ml) and extracted with EtOAc (2×25 mL). The combined organic layers were washed with brine (50 mL), dried over anhydrous Na₂SO₄ and concentrated under reduced pressure.

The crude product was purified by reversed phase column chromatography (Column, C18 silica gel, 40 g, 40-60 μm, 60 Å; mobile phase, water (0.05% TFA) and ACN (0% up to 40% ACN in 30 minutes); Detector, UV 220 & 254 nm) to afford tert-butyl N-(3-[[2-(3-methoxyphenyl)-1,3-thiazol-5-yl]formamido]propyl)carbamate (382 mg, 77%) as an off-white solid. LCMS (ES, m/z)⁺: 392 [M+H]⁺.

### Step 3. N-(3-aminopropyl)-2-(3-methoxyphenyl)-1,3-thiazole-5-carboxamide hydrochloride

A mixture of tert-butyl N-(3-[[2-(3-methoxyphenyl)-1,3-thiazol-5-yl]formamido]propyl)carbamate (380 mg, 0.99 mmol) in HCl in 1,4-dioxane (15 mL, 4M) was stirred for 1 hour at 25° C. The resulting mixture was concentrated under vacuum to afford N-(3-aminopropyl)-2-(3-methoxyphenyl)-1,3-thiazole-5-carboxamide hydrochloride (260 mg, 81%) as a white solid. LCMS (ES, m/z)⁺: 292 [M−HCl⁺H]⁺.

### Step 4. N-[3-(cyanoamino)propyl]-2-(3-methoxyphenyl)-1,3-thiazole-5-carboxamide (5)

To a stirred mixture of N-(3-aminopropyl)-2-(3-methoxyphenyl)-1,3-thiazole-5-carboxamide hydrochloride (100 mg, 0.30 mmol) in DMF (5.00 mL) was added NaHCO₃ (114 mg, 1.35 mmol) and BrCN (36 mg, 0.34 mmol) at 0° C. The resulting mixture was stirred for 1 hour at 25° C.

The mixture was purified by Prep-HPLC with the following conditions: Column, XBridge Prep OBD C18 Column, 30×150 mm, 5 μm; mobile phase, water (10 mmol/L NH₄HCO₃+0.1% NH₃.H₂O) and MeOH (20% Phase B up to 40% in 7 minutes); Detector, UV 254/220 nm. The product fraction was lyophilized to afford N-[3-(cyanoamino)propyl]-2-(3-methoxyphenyl)-1,3-thiazole-5-carboxamide (5) (24.3 mg, 28%) as a light yellow solid.

5¹H-NMR (400 MHz, DMSO-d6) δ (ppm): 8.78-8.75 (m, 1H), 8.43 (s, 1H), 7.56-7.54 (m, 1H), 7.51-7.50 (m, 1H), 7.49-7.42 (m, 1H), 7.13-7.10 (m, 1H), 6.76 (br s, 1H), 3.85 (s, 3H), 3.34-3.3.29 (m, 2H), 3.03-2.99 (m, 2H), 1.79-1.72 (m, 2H). LCMS (ES, m/z)⁺: 317 [M+H]⁺.

### Example 6

### Synthesis of 4-(cyanoamino)-N-[3-[4-(I-methylpyrazol-4-yl)phenyl]-1,2-oxazol-5-yl]butanamide (8)

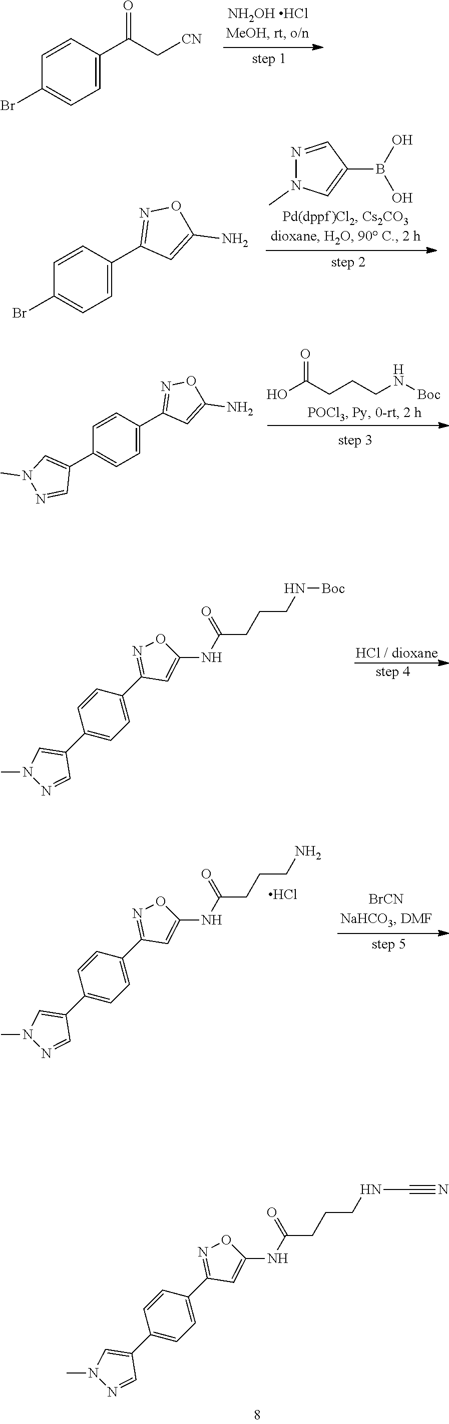

### Step 1. 3-(4-bromophenyl)-1,2-oxazol-5-amine

A mixture of 3-(4-bromophenyl)-3-oxopropanenitrile (20.0 g, 87.4 mmol), NH₂NH₂.HCl (8.98. g, 131 mmol) and NaOAc (10.8 g, 131 mmol) in MeOH (100 mL) was stirred overnight at 24° C. The mixture was diluted with water (300 mL) and extracted with EtOAc (3×300 mL). The combined organic layers were dried over anhydrous Na₂SO₄. After filtration, the filtrate was concentrated under reduced pressure to afford 3-(4-bromophenyl)-1,2-oxazol-5-amine (16.0 g, 73%) as a yellow solid. LCMS (ES, m/z)⁺: 239, 241 [M+H]⁺.

### Step 2. 3-[4-(1-methylpyrazol-4-yl)phenyl]-1,2-oxazol-5-amine

To a stirred mixture of 3-(4-bromophenyl)-1,2-oxazol-5-amine (10.0 g, 41.8 mmol) in dioxane (200 mL) was added 1-methylpyrazol-4-ylboronic acid (10.0 g, 79.4 mmol), Pd(dppf)Cl₂.CH₂Cl₂ (3.40 g, 4.16 mmol), Cs₂CO₃ (40.0 g, 123 mmol) and H₂O (60 mL). The resulting mixture was stirred for 6 hours at 90° C. under nitrogen atmosphere. The mixture was cooled to room temperature and concentrated under reduced pressure to remove dioxane. The precipitated solids were collected by filtration and washed with water (300 mL), MeOH (3×20 mL) and ethyl acetate (3×100 mL). This resulted in 3-[4-(1-methylpyrazol-4-yl)phenyl]-1,2-oxazol-5-amine (6.60 g, 53%) as a brown solid. LCMS (ES, m/z)⁺: 241 [M+H]⁺.

### Step 3. tert-butyl N-[3-([3-[4-(1-methylpyrazol-4-yl)phenyl]-1,2-oxazol-5-1]carbamoyl)propyl]carbamate

To a stirred solution of 3-[4-(1-methylpyrazol-4-yl)phenyl]-1,2-oxazol-5-amine (120 mg, 0.47 mmol) and 4-[(tert-butoxycarbonyl)amino]butanoic acid (146 mg, 0.71 mmol) in pyridine (3 mL) was added POCl₃ (147 mg, 0.95 mmol) dropwise at 0° C. The resulting mixture was stirred for 2 hours at 24° C. The mixture was diluted with ice/water (50 mL) and extracted with EtOAc (3×50 mL). The combined organic layers were dried over anhydrous Na2SO4 and concentrated under reduced pressure. The residue was purified by silica gel column chromatography, eluted with petroleum ether/EtOAc (1:2), to afford tert-butyl N-[3-([3-[4-(1-methylpyrazol-4-yl)phenyl]-1,2-oxazol-5-yl]carbamoyl)propyl]carbamate (80 mg, 37%) as a yellow solid. LCMS (ES, m/z)⁺: 426 [M+H]⁺.

### Step 4. 4-amino-N-[3-[4-(1-methylpyrazol-4-yl)phenyl]-1,2-oxazol-5-yl]butanamide hydrochloride

To a stirred solution of tert-butyl N-[3-([3-[4-(1-methylpyrazol-4-yl)phenyl]-1,2-oxazol-5-yl]carbamoyl)propyl]carbamate (80 mg, 0.18 mmol) in MeOH (1 mL) was added HCl in 1,4-dioxane (2 mL, 4M) dropwise at 24° C. The resulting mixture was stirred for 2 hours at 24° C. The resulting mixture was concentrated under vacuum to afford 4-amino-N-[3-[4-(1-methylpyrazol-4-yl)phenyl]-1,2-oxazol-5-yl]butanamide hydrochloride (60 mg, 88%) as a yellow solid. LCMS (ES, m/z)⁺: 326 [M−HCl⁺H]⁺.

### Step 5. 4-(cyanoamino)-N-[3-[4-(1-methylpyrazol-4-yl)phenyl]-1,2-oxazol-5-yl]butanamide (8)

To a stirred solution of 4-amino-N-[3-[4-(1-methylpyrazol-4-yl)phenyl]-1,2-oxazol-5-yl]butanamide (50 mg, 0.14 mmol) and NaHCO₃ (61 mg, 0.73 mmol) in DMF (1.5 mL) was added BrCN (17 mg, 0.16 mmol) in DMF (0.5 mL) dropwise at 0° C. The resulting mixture was stirred for 3 hours at 24° C. The mixture was diluted with water (50 mL) and extracted with EtOAc (3×50 mL). The combined organic layers were dried over anhydrous Na₂SO₄ and concentrated under reduced pressure.

The crude product was purified by Prep-HPLC with the following conditions: Column: XBridge Shield RP18 OBD Column, 5 μm, 19×150 mm; Mobile Phase A: water, Mobile Phase B: ACN; Flow rate: 25 mL/minute; Gradient: 40% B to 50% B in 7 minutes; 220/254 nm; Rt: 6.5 minutes. The product phase was lyophilized to afford 4-(cyanoamino)-N-[3-[4-(1-methylpyrazol-4-yl)phenyl]-1,2-oxazol-5-yl]butanamide (8) (20.3 mg, 39%) as a white solid.

8 ¹H-NMR (DMSO-d6, 400 MHz) δ (ppm): 11.7 (s, 1H), 8.24 (s, 1H), 7.95 (s, 1H), 7.82 (d, J=8.0 Hz, 2H), 7.69 (d, J=8.4 Hz, 2H), 6.81-6.74 (m, 2H), 3.88 (s, 3H), 3.02-2.97 (m, 2H), 2.50-2.34 (m, 2H), 1.85-1.78 (m, 2H). LCMS (ES, m/z)⁺: 351 [M+H]⁺.

### Example 7

### Synthesis of 4-(cyanoamino)-N-[2-(2-phenoxyphenyl)-1,3-thiazol-5-yl]butanamide (9)

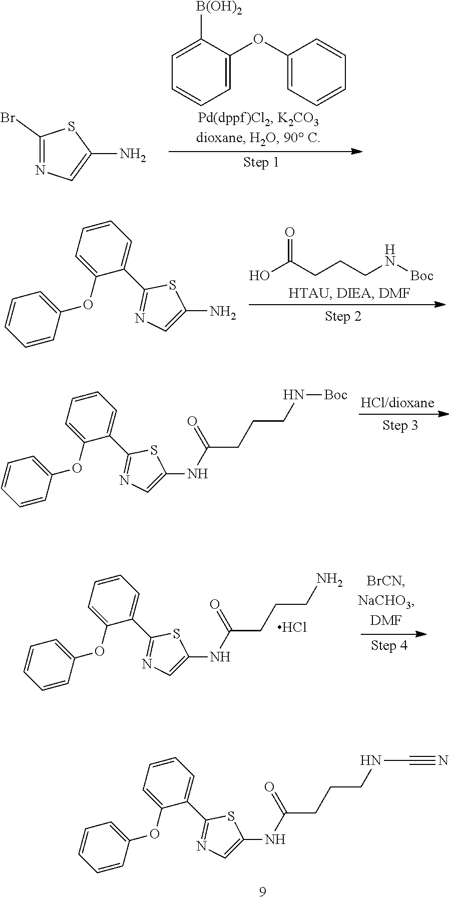

### Step 1. 2-(2-phenoxyphenyl)-1,3-thiazol-5-amine

To a stirred mixture of 2-bromo-1,3-thiazol-5-amine (500 mg, 2.79 mmol) and 2-phenoxyphenylboronic acid (597 mg, 2.79 mmol) in dioxane (10.00 mL) and H₂O (2.0 mL) was added Cs₂CO₃ (2.73 g, 8.38 mmol) and Pd(dppf)Cl₂ (408 mg, 0.56 mmol). The resulting mixture was stirred for 2 hours at 85° C. under a nitrogen atmosphere. The mixture was cooled to room temperature, diluted with water (20 mL) and extracted with EtOAc (3×20 mL). The combined organic layers were dried over anhydrous Na₂SO₄ and concentrated under reduced pressure. The residue was purified by silica gel column chromatography, eluted with petroleum ether/EtOAc (1:1), to afford 2-(2-phenoxyphenyl)-1,3-thiazol-5-amine (200 mg, 25%) as a white solid. LCMS (ES, m/z)⁺: 269 [M+H]⁺.

### Step 2. tert-butyl N-(3-{[2-(2-phenoxyphenyl)-1,3-thiazol-5-yl]carbamoyl}propyl) carbamate

To a stirred mixture of 2-(2-phenoxyphenyl)-1,3-thiazol-5-amine (200 mg, 0.74 mmol) and 4-[(tert-butoxycarbonyl)amino]butanoic acid (151 mg, 0.74 mmol) in DMF (3 mL) were added HATU (566 mg, 1.49 mmol) and DIEA (614 uL, 3.72 mmol). The resulting mixture was stirred for 2 hours at 25° C.

The mixture was purified by reversed phase column chromatography: Column, C18 silica gel, 40 g, 20-45 μm, 100 Å; mobile phase, water with 0.05% TFA and ACN (0% up to 60% ACN in 40 minutes); Detector, UV 220 & 254 nm. The product fraction was concentrated under reduced pressure to afford tert-butyl N-(3-{[2-(2-phenoxyphenyl)-1,3-thiazol-5-yl]carbamoyl}propyl)carbamate (150 mg, 42%) as a white solid. LCMS (ES, m/z)⁺: 454[M+H]⁺.

### Step 3. 4-amino-N-[2-(2-phenoxyphenyl)-1,3-thiazol-5-yl]butanamide hydrochloride

A mixture of tert-butyl N-(3-[[2-(2-phenoxyphenyl)-1,3-thiazol-5-yl]carbamoyl]propyl)carbamate (140 mg, 0.31 mmol) in HCl(gas) in 1,4-dioxane (3 mL) was stirred for 1 hour at 25° C. The resulting mixture was concentrated under vacuum to afford 4-amino-N-[2-(2-phenoxyphenyl)-1,3-thiazol-5-yl]butanamide hydrochloride (100 mg, 82%) as a white solid. LCMS (ES, m/z)⁺: 354[M−HCl⁺H]⁺.

### Step 4. 4-(cyanoamino)-N-[2-(2-phenoxyphenyl)-1,3-thiazol-5-yl]butanamide (9)

990 To a stirred mixture of 4-amino-N-[2-(2-phenoxyphenyl)-1,3-thiazol-5-yl]butanamide hydrochloride (70 mg, 0.18 mmol) and NaHCO₃ (45 mg, 0.53 mmol) in DMF (1.50 mL) was added BrCN (19 mg, 0.18 mmol) at 0° C. The resulting mixture was stirred for 2 hours at 25° C.

The mixture was purified by Prep-HPLC with the following conditions: Column, XBridge Shield RP18 OBD Column, 5 μm, 19×150 mm; mobile phase, water with 10 mM NH4HCO3 and ACN (40% Phase B up to 60% in 10 minutes); Detector, UV254/220 nm. The collected fraction was lyophilized to afford 4-(cyanoamino)-N-[2-(2-phenoxyphenyl)-1,3-thiazol-5-yl]butanamide (9) (26 mg, 37%) as a white solid.

9 ¹H-NMR (DMSO-d6, 400 MHz) δ (ppm): 12.11 (s, 1H), 7.98 (s, 1H), 7.88-7.85 (m, 1H), 7.41-7.37 (m, 2H), 7.34-7.30 (m, 1H), 7.26-7.22 (m, 1H), 7.16-7.12 (m, 1H), 7.00-6.97 (m, 3H), 6.78-6.76 (m, 1H), 2.98-2.93 (m, 2H), 2.47-2.39 (m, 2H), 1.81-1.74 (m, 2H). LCMS (ES, m/z)⁺: 379 [M+H]⁺.

### Example 8

### Synthesis of 4-(cyanoamino)-N-[2-(2-methoxyphenyl)-1,3-thiazol-5-yl]butanamide (10)

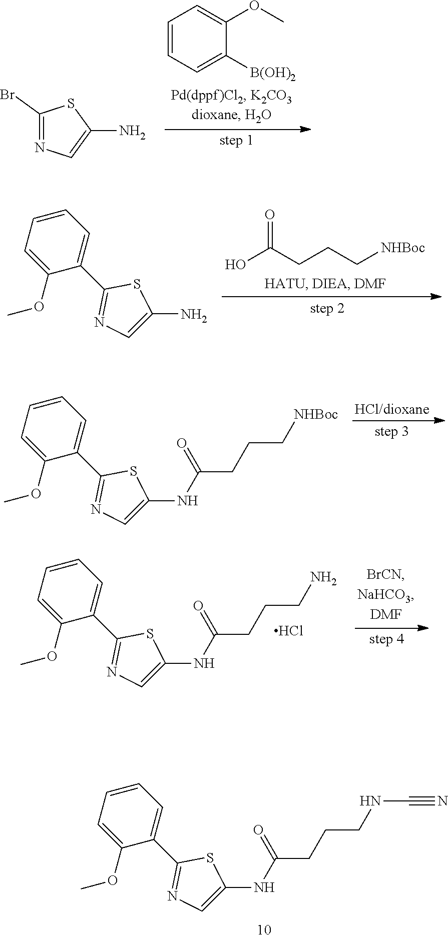

### Step 1. 2-(2-methoxyphenyl)-1,3-thiazol-5-amine

To a stirred mixture of 2-bromo-1,3-thiazol-5-amine (300 mg, 1.67 mmol) and 2-methoxyphenylboronic acid (374 mg, 2.46 mmol) in dioxane (10 mL) and H2O (2 mL) were added Cs₂CO₃ (1.64 g, 5.00 mmol) and Pd(dppf)Cl₂ (245 mg, 0.33 mmol) under a nitrogen atmosphere. The resulting mixture was stirred for 2 hours at 80° C. under a nitrogen atmosphere. The mixture was cooled to room temperature, poured into water (30 mL) and extracted with CH₂C₂ (3×15 mL). The combined organic layers were washed with brine (60 mL) and dried over anhydrous Na₂SO₄. After filtration, the filtrate was concentrated under reduced pressure. The residue was purified by silica gel column chromatography, eluted with petroleum ether/EtOAc (1:1), to afford 2-(2-methoxyphenyl)-1,3-thiazol-5-amine (100 mg, 27%) as a white solid. LCMS (ES, m/z)⁺: 207 [M+H]⁺.

### Step 2. tert-butyl N-(3-[[2-(2-methoxyphenyl)-1,3-thiazol-5-yl]carbamoyl]propyl) carbamate

To a stirred mixture of 2-(2-methoxyphenyl)-1,3-thiazol-5-amine (80 mg, 0.38 mmol) and 4-[(tert-butoxycarbonyl)amino]butanoic acid (115 mg, 0.56 mmol) in DMF (2 mL) was added HATU (219 mg, 0.57 mmol) and DIEA (256 uL, 1.54 mmol). The resulting mixture was stirred for 1 hour at 22° C. The resulting mixture was poured into water (30 mL) and extracted with ethyl acetate (3×15 mL). The combined organic layers were washed with brine (60 mL) and dried over anhydrous Na₂SO₄. After filtration, the filtrate was concentrated under reduced pressure.

The crude product was purified by reverse phase column (Column: C18 column, 120 g, 20-35 μm; Mobile Phase: water (0.05% TFA) and ACN (0% B to 60% B in 30 minutes); Detector: UV 254/220 nm) to afford tert-butyl N-(3-[[2-(2-methoxyphenyl)-1,3-thiazol-5-yl]carbamoyl]propyl)carbamate (130 mg, 81%) as a yellow oil. LCMS (ES, m/z)⁺: 392 [M+H]⁺.

### Step 3. 4-amino-N-[2-(2-methoxyphenyl)-1,3-thiazol-5-yl]butanamide hydrochloride

A mixture of tert-butyl N-(3-[[2-(2-methoxyphenyl)-1,3-thiazol-5-yl]carbamoyl]propyl)carbamate (100 mg, 0.25 mmol) and HCl(gas) in 1,4-dioxane (3 mL) was stirred for 1 hour at 22° C. The resulting mixture was concentrated under reduced pressure to afford 4-amino-N-[2-(2-methoxyphenyl)-1,3-thiazol-5-yl]butanamide hydrochloride (80 mg, 95%) as a white solid. LCMS (ES, m/z)⁺: 292 [M−HCl⁺H]⁺.

### Step 4. 4-(cyanoamino)-N-[2-(2-methoxyphenyl)-1,3-thiazol-5-yl]butanamide (10)

To a stirred mixture of 4-amino-N-[2-(2-methoxyphenyl)-1,3-thiazol-5-yl]butanamide hydrochloride (40 mg, 0.12 mmol) and NaHCO₃ (40 mg, 0.47 mmol) in DMF (1 mL) was added BrCN (1.20 mL, 0.11 mmol, 10 mg/mL in DMF) at 0° C. The resulting mixture was stirred for 1 hour at 22° C.

The mixture was purified by Prep-HPLC with the following conditions: Column: XBridge Shield RP18 OBD Column 30×150 mm, 5 μm; Mobile Phase A: water (10 mmol/L NH₄HCO₃), Mobile Phase B: ACN; Flow rate: 25 mL/minute; Gradient: 35% B to 60% B in 7 minutes; 254/220 nm) The product fractions were pooled and lyophilized to afford 4-(cyanoamino)-N-[2-(2-methoxyphenyl)-1,3-thiazol-5-yl]butanamide (10) (12 mg, 29%) as a white solid.

10 ¹H-NMR (DMSO-d6, 400 MHz) δ (ppm): 12.06 (s, 1H), 7.91 (s, 1H), 7.70-7.67 (m, 1H), 7.32-7.28 (m, 1H), 7.14 (d, J=8.0 Hz, 1H), 7.03-6.99 (m, 1H), 6.81-6.78 (m, 1H), 3.91 (s, 3H), 3.01-2.96 (m, 2H), 2.54-2.52 (m, 2H), 1.85-1.78 (m, 2H). LCMS (ES, m/z)⁺: 317 [M+H]⁺.

### Example 9

### Synthesis of cyano([[(3S)-1-[2-(2-methoxyphenyl)-1,3-thiazole-4-carbonyl]pyrrolidin-3-yl]methyl])amine (12)

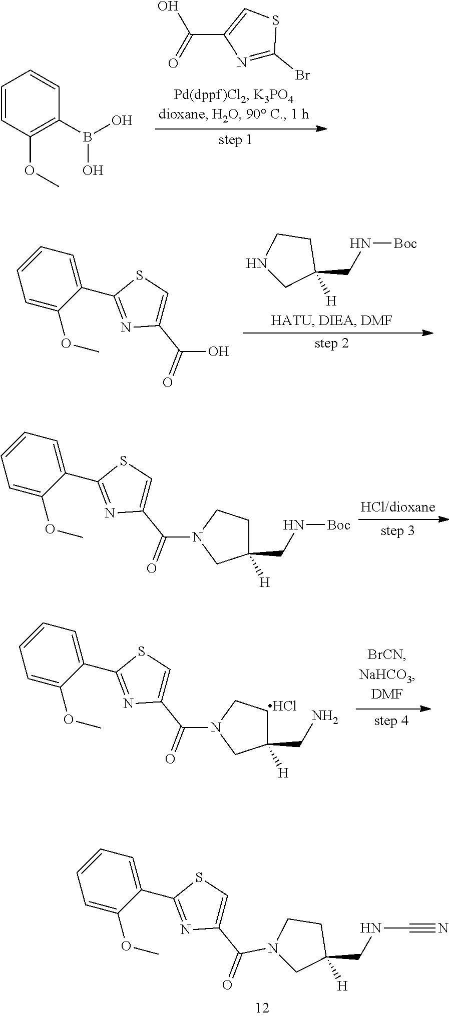

### Step 1. 2-(2-methoxyphenyl)-1,3-thiazole-4-carboxylic acid

A mixture of 2-methoxyphenylboronic acid (2.20 g, 14.5 mmol), 2-bromo-1,3-thiazole-4-carboxylic acid (2.00 g, 9.61 mmol), Pd(dppf)Cl₂ (1.40 g, 1.91 mmol) and K₃PO₄ (6.10 g, 28.7 mmol) in dioxane (60 mL) and H₂O (30 mL) was stirred for 1 hour at 90° C. under nitrogen atmosphere. The reaction mixture was cooled to room temperature poured into water/ice and extracted with CH₂C₂ (3×100 mL). The aqueous layer was concentrated under reduced pressure.

The residue was purified by reverse flash chromatography with the following conditions: column, C18 silica gel, 330 g, 20-35 μm; mobile phase, water with TFA (0.05%) and ACN (0% to 100% gradient in 25 minutes); detector, UV 254/220 nm. The product fraction was concentrated under reduced pressure to afford 2-(2-methoxyphenyl)-1,3-thiazole-4-carboxylic acid (470 mg, 20%) as a yellow solid. LCMS (ES, m/z)⁺: 236 [M+H]⁺.

### Step 2. tert-butyl N-[[(3S)-1-[2-(2-methoxyphenyl)-1,3-thiazole-4-carbonyl]pyrrolidin-3-yl]methyl]carbamate

To a stirred mixture of 2-(2-methoxyphenyl)-1,3-thiazole-4-carboxylic acid (250 mg, 1.06 mmol) and HATU (606 mg, 1.59 mmol) in DMF (5 mL) was added DIEA (527 uL, 3.19 mmol) and tert-butyl N-[(3R)-pyrrolidin-3-ylmethyl]carbamate (235 mg, 1.17 mmol) at 0° C.

The resulting mixture was stirred for 2 hours at 25° C. The mixture was purified by reverse flash chromatography with the following conditions: column, C18 silica gel, 80 g, 20-35 μm; mobile phase, water with NH₄HCO₃ (10 mmol/L) and ACN (0% to 60% gradient in 15 minutes); detector, UV 254/220 nm. The collected fraction was concentrated under reduced pressure to afford tert-butyl N-[[(3 S)-1-[2-(2-methoxyphenyl)-1,3-thiazole-4-carbonyl]pyrrolidin-3-yl]methyl]carbamate (420 mg, 94%) as a light brown solid. LCMS (ES, m/z)⁺: 418 [M+H]⁺.

### Step 3. 1-[(3S)-1-[2-(2-methoxyphenyl)-1,3-thiazole-4-carbonyl]pyrrolidin-3-yl]methanamine hydrochloride

To a stirred mixture of tert-butyl N-[[(3S)-1-[2-(2-methoxyphenyl)-1,3-thiazole-4-carbonyl]pyrrolidin-3-yl]methyl]carbamate (400 mg, 0.96 mmol) in DCM (10 mL) was added HCl in 1,4-dioxane (5 mL, 4M) dropwise at 0° C. The resulting mixture was stirred for 1 hour at 25° C. The mixture was concentrated under reduced pressure to afford 1-[(3S)-1-[2-(2-methoxyphenyl)-1,3-thiazole-4-carbonyl]pyrrolidin-3-yl]methanamine hydrochloride (330 mg, 98%) as an off-white solid. LCMS (ES, m/z)⁺: 318 [M−HCl⁺H]⁺.

### Step 4. cyano([[(3S)-1-[2-(2-methoxyphenyl)-1,3-thiazole-4-carbonyl]pyrrolidin-3-yl]methyl])amine (12)

To a stirred mixture of 1-[(3S)-1-[2-(2-methoxyphenyl)-1,3-thiazole-4-carbonyl]pyrrolidin-3-yl]methanamine hydrochloride (200 mg, 0.57 mmol) in DMF (5 mL) was added NaHCO₃ (238 mg, 2.83 mmol) and BrCN (59 mg, 0.56 mmol) dropwise at 0° C. The resulting mixture was stirred for 2 hours at 25° C.

The mixture was purified by Prep-HPLC with the following conditions: Column, XBridge Shield RP18 OBD Column, 5 μm, 19×150 mm; mobile phase, water and ACN (25% up to 50% in 7 minutes); Detector, UV220/254 nm. The collected fraction was lyophilized to afford cyano([[(3S)-1-[2-(2-methoxyphenyl)-1,3-thiazole-4-carbonyl]pyrrolidin-3-yl]methyl])amine (12) (31.9 mg, 16%) as a white solid.

12 ¹H NMR (400 MHz, DMSO-d6) δ (ppm): 8.34-8.24 (m, 2H), 7.53-7.49 (m, 1H), 7.28 (d, J=8.4 Hz, 1H), 7.16-7.11 (m, 1H), 6.94-6.90 (m, 1H), 4.17-4.07 (m, 1H), 4.04 (s, 3H), 3.80-3.53 (m, 2H), 3.32-3.25 (m, 1H), 3.07-3.02 (m, 2H), 2.45-2.40 (m, 1H), 2.16-1.98 (m, 1H), 1.73-1.66 (m, 1H). LCMS (ES, m/z)⁺: 343 [M+H]⁺.

### Example 10

### Synthesis of [(6-[3-[4-(1-methyl-1H-pyrazol-4-yl)phenyl]-1,2-oxazole-5-carbonyl]-6-azaspiro[3.4]octan-2-yl)amino]formonitrile (13)

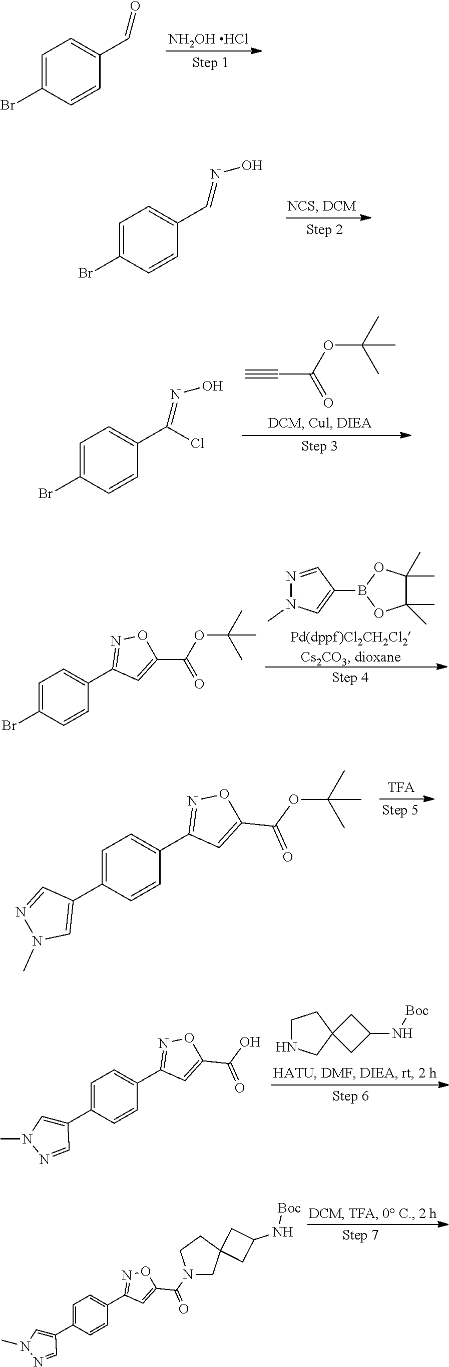

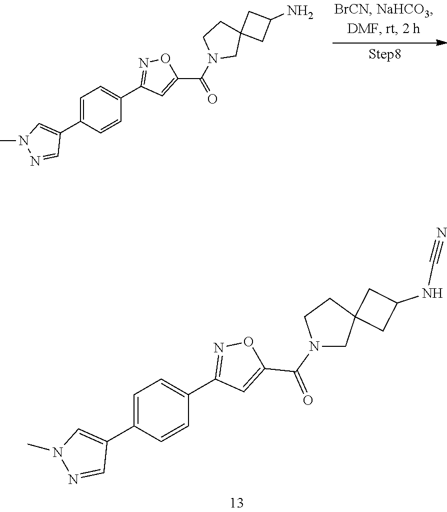 [SMILES: Cn1cc(-c2ccc(-c3cc(C(=O)N4CCC5(CC(N)C5)C4)on3)cc2)cn1 ; Cn1cc(-c2ccc(-c3cc(C(=O)N4CCC5(CC(NC#N)C5)C4)on3)cc2)cn1]

### Step 1. N-[(4-bromophenyl)methylidene]hydroxylamine

To the mixture of 4-bromobenzaldehyde (5.00 g, 27.2 mmol) in EtOH (40 mL) was added hydroxylamine hydrochloride (2.20 g, 31.9 mmol) at room temperature. The resulting mixture was stirred for 12 hour at room temperature. The resulting mixture was concentrated under reduced pressure. The residue was diluted with water (200 mL), the solids was collected by filtration and washed with water (3×50 mL) to afford N-[(4-bromophenyl)methylidene]hydroxylamine (4.40 g, 81%) as a white solid. LCMS (ES, m/z)⁺: 200, 202 [M+H]⁺.

### Step 2. 4-bromo-N-hydroxybenzene-1-carbonimidoyl chloride

To a mixture of N-[(4-bromophenyl)methylidene]hydroxylamine (4.00 g, 20.1 mmol) in DMF (40 mL), NCS (3.20 g, 24.0 mmol) was added in portions at 0° C. The resulting mixture was stirred for 4 hours at room temperature. The mixture was diluted with water (100 mL) and extracted with DCM (3×100 mL). The combined organic layers were washed with brine (50 mL) and dried over anhydrous Na2SO₄. After filtration, the filtrate was concentrated under reduced pressure to afford 4-bromo-N-hydroxybenzene-1-carbonimidoyl chloride (4.00 g, 85%) as a yellow oil. LCMS (ES, m/z)⁺: 234, 236, 238 [M+H]⁺.

### Step 3. tert-butyl 3-(4-bromophenyl)-1,2-oxazole-5-carboxylate

Tert-butyl prop-2-ynoate (2.50 g, 19.8 mmol), CuI (500 mg, 2.63 mmol) and DIEA (7.68 mL, 46.4 mmol), were added at room temperature to a mixture of 4-bromo-N-hydroxybenzene-1-carbonimidoyl chloride (3.90 g, 16.7 mmol) in DCM (40 mL). The resulting mixture was stirred for 12 hours at room temperature under nitrogen atmosphere. The mixture was diluted with water (100 mL) and extracted with DCM (3×100 mL). The combined organic layers were washed with brine (50 mL) and dried over anhydrous Na₂SO₄. After filtration, the filtrate was concentrated under reduced pressure. The residue was purified by silica gel column chromatography, eluted with PE/EtOAc (1:1), to afford tert-butyl 3-(4-bromophenyl)-1,2-oxazole-5-carboxylate (3.00 g, 56%) as a yellow oil. LCMS (ES, m/z)⁺: 324, 326 [M+H]⁺.

### Step 4. tert-butyl 3-[4-(1-methyl-1H-pyrazol-4-yl)phenyl]-1,2-oxazole-5-carboxylate

A mixture of tert-butyl 3-(4-bromophenyl)-1,2-oxazole-5-carboxylate (2.70 g, 8.36 mmol), 1-methyl-4-(4,4,5,5-tetramethyl-1,3,2-dioxaborolan-2-yl)-1H-pyrazole (2.00 g, 9.60 mmol), Pd(dppf)Cl₂ (330 mg, 0.41 mmol) and Cs2CO3 (8.0 g, 24.5 mmol) in dioxane (20 mL) and H2O (2 mL) was stirred for 12 hours at 90° C. under nitrogen atmosphere. The mixture was cooled to room temperature, diluted with water (50 mL) and extracted with EtOAc (3×50 mL). The combined organic layers were washed with brine (50 mL) and dried over anhydrous Na₂SO₄. After filtration, the filtrate was concentrated under reduced pressure. The residue was purified by silica gel column chromatography, eluted with hexane/EtOAc (1:1), to afford tert-butyl 3-[4-(1-methyl-1H-pyrazol-4-yl)phenyl]-1,2-oxazole-5-carboxylate (2.20 g, 81%) as a brown solid. LCMS (ES, m/z)⁺: 326 [M+H]⁺.

### Step 5. 3-[4-(1-methyl-1H-pyrazol-4-yl)phenyl]-1,2-oxazole-5-carboxylic acid

To a stirred mixture of tert-butyl 3-[4-(1-methyl-1H-pyrazol-4-yl)phenyl]-1,2-oxazole-5-carboxylate (2.20 g, 6.77 mmol) in DCM (20 mL) was added TFA (2 mL) dropwise at 0° C. The resulting mixture was stirred for 2 hours at room temperature. The mixture was concentrated to dryness under vacuum. The residue was washed with DCM (3×5 mL) to afford 3-[4-(1-methyl-1H-pyrazol-4-yl)phenyl]-1,2-oxazole-5-carboxylic acid (1.40 g, 77%) as a white solid. LCMS (ES, m/z)⁺: 270 [M+H]⁺.

### Step 6. tert-butyl N-(6-[3-[4-(1-methyl-1H-pyrazol-4-yl)phenyl]-1,2-oxazole-5-carbonyl]-6-azaspiro[3.4]octan-2-yl)carbamate

A mixture of 3-[4-(1-methyl-1H-pyrazol-4-yl)phenyl]-1,2-oxazole-5-carboxylic acid (100 mg, 0.36 mmol), HATU (207 mg, 0.54 mmol), tert-butyl N-[6-azaspiro[3.4]octan-2-yl]carbamate (99 mg, 0.44 mmol), DIEA (180 uL, 1.09 mmol) in DMF (5 mL) was stirred for 2 hours at room temperature. The mixture was slowly poured into water (100 mL) and the precipitated solids were collected by filtration to afford tert-butyl N-(6-[3-[4-(1-methyl-1H-pyrazol-4-yl)phenyl]-1,2-oxazole-5-carbonyl]-6-azaspiro[3.4]octan-2-yl)carbamate (75 mg, 41%) as a white solid. LCMS (ES, m/z)⁺: 478 [M+H]⁺.

### Step 7. 6-[3-[4-(1-methyl-1H-pyrazol-4-yl)phenyl]-1,2-oxazole-5-carbonyl]-6-azaspiro[3.4]octan-2-amine

A solution of tert-butyl N-(6-[3-[4-(1-methyl-1H-pyrazol-4-yl)phenyl]-1,2-oxazole-5-carbonyl]-6-azaspiro[3.4]octan-2-yl)carbamate (75 mg, 0.15 mmol) and TFA (1 mL) in DCM (5 mL) was stirred for 2 hours at 0° C. The mixture was concentrated under vacuum. The residue was purified by reverse flash chromatography with the following conditions: Column: C18 silica gel, 40 g; Mobile phase: water (containing 0.05% NH₄HCO₃) and ACN (0% to 80% in 30 min); Detector: UV 254/220 nm). The collected fraction was concentrated under vacuum to afford 6-[3-[4-(1-methyl-1H-pyrazol-4-yl)phenyl]-1,2-oxazole-5-carbonyl]-6-azaspiro[3.4]octan-2-amine (50 mg, 84%) as a white solid. LCMS (ES, m/z)⁺: 378[M+H]⁺.

### Step 8. [(6-[3-[4-(1-methyl-1H-pyrazol-4-yl)phenyl]-1,2-oxazole-5-carbonyl]-6-azaspiro[3.4]octan-2-yl)amino]formonitrile (13)

To a stirred mixture of 6-[3-[4-(1-methyl-1H-pyrazol-4-yl)phenyl]-1,2-oxazole-5-carbonyl]-6-azaspiro[3.4]octan-2-amine (50 mg, 0.12 mmol) and NaHCO₃ (40 mg, 0.48 mmol) in DMF (5 mL) was added BrCN (15 mg, 0.14 mmol) at 0° C. The reaction mixture was stirred for 2 hours at 0° C.

The mixture was purified by reverse flash chromatography with the following conditions: Column: C18 silica gel, 40 g, 20-35 μm; Mobile phase: water (containing 0.05% NH₄HCO₃) and ACN (0% to 70% in 30 minutes); Detector: UV 254/220 nm. The collected fraction was lyophilized to afford [(6-[3-[4-(1-methyl-1H-pyrazol-4-yl)phenyl]-1,2-oxazole-5-carbonyl]-6-azaspiro[3.4]octan-2-yl)amino]formonitrile (13) (20 mg, 40%) as a white solid.

13 ¹H NMR (400 MHz, DMSO-d6) δ (ppm): 8.27 (s, 1H), 7.98 (s, 1H), 7.96-7.93 (m, 2H), 7.75-7.72 (m, 2H), 7.65 (d, J=5.2 Hz, 1H), 7.19-7.13 (m, 1H), 3.89 (s, 3H), 3.87-3.79 (m, 1H), 3.75-6.68 (m, 2H), 3.56-3.54 (m, 1H), 3.48 (s, 1H), 2.36-2.23 (m, 2H), 2.04-2.00 (m, 3H), 1.99-1.90 (m, 1H). LCMS (ES, m/z)⁺: 403 [M+H]⁺.

### Example 11

### Synthesis of cyano([[(3R)-1-[2-(2-methoxyphenyl)-1,3-thiazole-4-carbonyl]pyrrolidin-3-yl]methyl])amine (14)

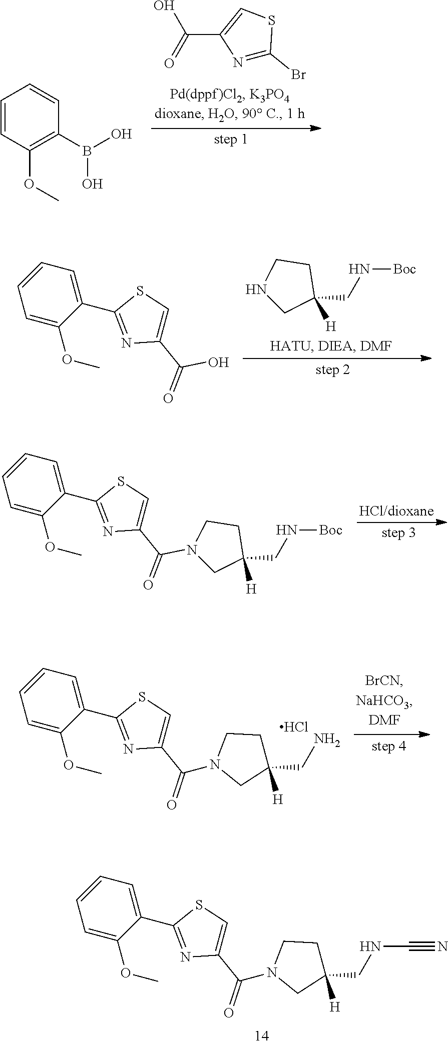

### Step 1. 2-(2-methoxyphenyl)-1,3-thiazole-4-carboxylic acid

A mixture of 2-methoxyphenylboronic acid (2.20 g, 14.5 mmol), 2-bromo-1,3-thiazole-4-carboxylic acid (2.00 g, 9.61 mmol), Pd(dppf)Cl₂ (1.40 g, 1.91 mmol) and K₃PO₄ (6.10 g, 28.7 mmol) in dioxane (60 mL) and H₂O (30 mL) was stirred for 1 hour at 90° C. under nitrogen atmosphere. The reaction mixture was cooled to room temperature and poured into water/ice and extracted with CH₂C₂ (3×100 mL). The aqueous layer was concentrated under reduced pressure.

The residue was purified by reverse flash chromatography with the following conditions: column, C18 silica gel, 330 g, 20-35 μm; mobile phase, water with TFA (0.05%) and ACN (0% to 100% gradient in 25 minutes); detector, UV 254/220 nm. The product fraction was concentrated under reduced pressure to afford 2-(2-methoxyphenyl)-1,3-thiazole-4-carboxylic acid (470 mg, 20%) as a yellow solid. LCMS (ES, m/z)⁺: 236 [M+H]⁺.

### Step 2. tert-butyl N-[[(3R)-1-[2-(2-methoxyphenyl)-1,3-thiazole-4-carbonyl]pyrrolidin-3-yl]methyl]carbamate

To a stirred mixture of 2-(2-methoxyphenyl)-1,3-thiazole-4-carboxylic acid (200 mg, 0.85 mmol) and HATU (485 mg, 1.28 mmol) in DMF (6 mL) was added DIEA (421 μL, 2.55 mmol) and tert-butyl N-[(3S)-pyrrolidin-3-ylmethyl]carbamate (206 mg, 1.03 mmol) at 0° C. The resulting mixture was stirred for 2 hours at 25° C. under nitrogen atmosphere. The mixture was purified by reverse flash chromatography with the following conditions: column, C18 silica gel, 120 g, 20-35 μm; mobile phase, water with NH₄HCO₃ (10 mmol/L) and ACN (0% to 60% gradient in 25 min); detector, UV 254/220 nm. The collected fractions were concentrated under reduced pressure to afford tert-butyl N-[[(3R)-1-[2-(2-methoxyphenyl)-1,3-thiazole-4-carbonyl]pyrrolidin-3-yl]methyl]carbamate (360 mg, 96%, 95% purity) as a brown oil. LCMS (ES, m/z)⁺: 418 [M+H]⁺.

### Step 3. 1-[(3R)-1-[2-(2-methoxyphenyl)-1,3-thiazole-4-carbonyl]pyrrolidin-3-yl]methanamine hydrochloride

To a stirred mixture of tert-butyl N-[[(3R)-1-[2-(2-methoxyphenyl)-1,3-thiazole-4-carbonyl]pyrrolidin-3-yl]methyl]carbamate (200 mg, 0.48 mmol) in DCM (10 mL) was added HCl in 1,4-dioxane (5 mL, 4M) dropwise at 0° C. under nitrogen atmosphere. The resulting mixture was stirred for 2 hours at 25° C. The resulting mixture was concentrated under reduced pressure to afford 1-[(3R)-1-[2-(2-methoxyphenyl)-1,3-thiazole-4-carbonyl]pyrrolidin-3-yl]methanamine hydrochloride (170 mg, 95%) as an off-white solid. LCMS (ES, m/z)⁺: 318 [M−HCl+H]⁺.

### Step 4. cyano([[(3R)-1-[2-(2-methoxyphenyl)-1,3-thiazole-4-carbonyl]pyrrolidin-3-yl]methyl])amine (14)

To a stirred mixture of 1-[(3R)-1-[2-(2-methoxyphenyl)-1,3-thiazole-4-carbonyl]pyrrolidin-3-yl]methanamine hydrochloride (100 mg, 0.28 mmol) in DMF (5 mL) was added NaHCO₃ (119 mg, 1.42 mmol) and BrCN (27 mg, 0.26 mmol) at 0° C. The resulting mixture was stirred for 1 hour at 25° C.

The mixture was purified by Prep-HPLC with the following conditions: Column, XBridge Shield RP18 OBD Column, 5 μm, 19×150 mm; mobile phase, water and ACN (20% up to 40% in 7 minutes); Detector, UV220/254 nm. The collected fraction was lyophilizied to afford cyano([[(3R)-1-[2-(2-methoxyphenyl)-1,3-thiazole-4-carbonyl]pyrrolidin-3-yl]methyl])amine (14) (26.6 mg, 26%) as a white solid.

14 ¹H NMR (400 MHz, DMSO-d6) δ (ppm): 8.34-8.25 (m, 2H), 7.53-7.49 (m, 1H), 7.28 (d, J=8.4 Hz, 1H), 7.16-7.11 (m, 1H), 6.94-6.90 (m, 1H), 4.16-4.07 (m, 1H), 4.05 (s, 3H), 3.80-3.50 (m, 2H), 3.33-3.25 (m, 1H), 3.07-3.02 (m, 2H), 2.47-2.39 (m, 1H), 2.15-1.97 (m, 1H), 1.73-1.66 (m, 1H). LCMS (ES, m/z)⁺: 343 [M+H]⁺.

### Example 12

### Synthesis of 3-(cyanoamino)-N-[3-[4-(I-methylpyrazol-4-yl)phenyl]1,2-oxazol-5-yl]propanamide (15)

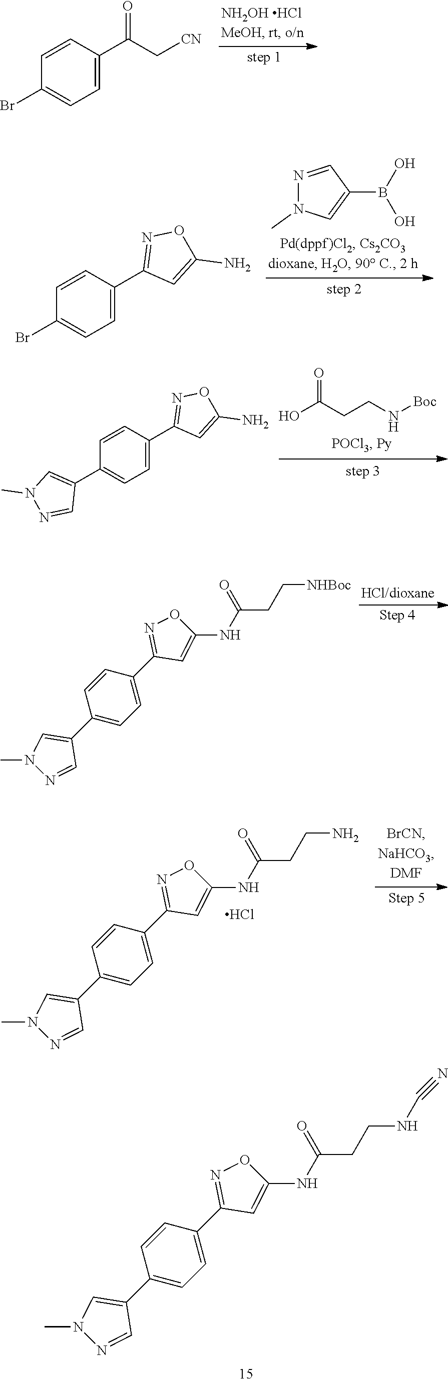

### Step 1. 3-(4-bromophenyl)-1,2-oxazol-5-amine

A mixture of 3-(4-bromophenyl)-3-oxopropanenitrile (20.0 g, 87.4 mmol), NH₂NH₂.HCl (8.98. g, 131 mmol) and NaOAc (10.8 g, 131 mmol) in MeOH (100 mL) was stirred overnight at 24° C. The mixture was diluted with water (300 mL) and extracted with EtOAc (3×300 mL). The combined organic layers were dried over anhydrous Na₂SO₄. After filtration, the filtrate was concentrated under reduced pressure to afford 3-(4-bromophenyl)-1,2-oxazol-5-amine (16.0 g, 73%) as a yellow solid. LCMS (ES, m/z)⁺: 239, 241 [M+H]⁺.

### Step 2. 3-[4-(1-methylpyrazol-4-yl)phenyl]-1,2-oxazol-5-amine

To a stirred mixture of 3-(4-bromophenyl)-1,2-oxazol-5-amine (10.0 g, 41.8 mmol) in dioxane (200 mL) was added 1-methylpyrazol-4-ylboronic acid (10.0 g, 79.4 mmol), Pd(dppf)Cl₂.CH₂Cl₂ (3.40 g, 4.16 mmol), Cs2CO3 (40.0 g, 123 mmol) and H₂O (60 mL). The resulting mixture was stirred for 6 hours at 90° C. under a nitrogen atmosphere. The mixture was cooled to room temperature and concentrated under reduced pressure to remove dioxane. The precipitated solids were collected by filtration and washed with water (300 mL), MeOH (3×20 mL) and ethyl acetate (3×100 mL). This resulted in 3-[4-(1-methylpyrazol-4-yl)phenyl]-1,2-oxazol-5-amine (6.60 g, 53%) as a brown solid. LCMS (ES, m/z)⁺: 241 [M+H]⁺.

### Step 3. Tert-butyl N-[2-([3-[4-(1-methylpyrazol-4-yl)phenyl]-1,2-oxazol-5-yl]carbamoyl)ethyl]carbamate

To a stirred mixture of 3-[4-(1-methylpyrazol-4-yl)phenyl]-1,2-oxazol-5-amine (150 mg, 0.74 mmol) and 3-[(tert-butoxycarbonyl)amino]propanoic acid (140 mg, 0.74 mmol) in pyridine (5 mL) was added POCl₃ (241 mg, 1.49 mmol) dropwise at 0° C. under a nitrogen atmosphere. The resulting mixture was stirred for 2 hours at 25° C. under nitrogen atmosphere. The reaction mixture was diluted with water/ice (30 mL) and extracted with ethyl acetate (3×30 mL). The combined organic layers were dried over anhydrous Na₂SO₄ and concentrated under reduced pressure. The residue was purified by silica gel column chromatography (eluting with 3:1 ethyl acetate/petroleum ether) to afford tert-butyl N-[2-([3-[4-(1-methylpyrazol-4-yl)phenyl]-1,2-oxazol-5-yl]carbamoyl)ethyl]carbamate as a white solid (150 mg, 58%). LCMS (ES, m/z)⁺: 412 [M+H]⁺.

### Step 4. 3-amino-N-[3-[4-(1-methylpyrazol-4-yl)phenyl]-1,2-oxazol-5-yl]propanamide hydrogen chloride

To a stirred mixture of tert-butyl N-[2-([3-[4-(1-methylpyrazol-4-yl)phenyl]-1,2-oxazol-5-yl]carbamoyl)ethyl]carbamate (140 mg, 0.30 mmol) in DCM (3 mL) was added HCl in 1,4-dioxane (3 mL, 4M) dropwise at 0° C. under a nitrogen atmosphere. The resulting mixture was stirred for 2 hours at 25° C. under a nitrogen atmosphere. The resulting mixture was concentrated under reduced pressure to afford 3-amino-N-[3-[4-(1-methylpyrazol-4-yl)phenyl]-1,2-oxazol-5-yl]propanamide hydrogen chloride as a white solid (100 mg, 84%). LCMS (ES, m/z)⁺: 312 [M−HCl+H]⁺.

### Step 5. 3-(cyanoamino)-N-[3-[4-(1-methylpyrazol-4-yl)phenyl]-1,2-oxazol-5-yl]propanamide (15)

To a stirred mixture of 3-amino-N-[3-[4-(1-methylpyrazol-4-yl)phenyl]-1,2-oxazol-5-yl]propanamide (100 mg, 0.29 mmol) in DMF (10 mL) was added NaHCO₃ (121 mg, 1.44 mmol) and cyanogen bromide (31 mg, 0.28 mmol) at 0° C. The resulting mixture was stirred for 2 hours at 25° C. The mixture diluted with ice/water (20 mL) and extracted with DCM (2×20 mL). The combined organic layers were dried over anhydrous sodium sulfate and concentrated under reduced pressure.

The residue was purified by Prep-HPLC with the following conditions: Column, XBridge Shield RP18 OBD Column, 5 μm, 19×150 mm; mobile phase, H2O and ACN (35% Phase B up to 40% in 7 minutes); Detector, UV 254/220 nm. The collected fraction was lyophilized to afford 3-(cyanoamino)-N-[3-[4-(1-methylpyrazol-4-yl)phenyl]-1,2-oxazol-5-yl]propanamide (15) as a white solid (11.5 mg, 11%).

15 ¹H NMR (400 MHz, DMSO-d6) δ (ppm): 11.83 (s, 1H), 8.24 (s, 1H), 7.95 (s, 1H), 7.83 (d, J=8.0 Hz, 2H), 7.69 (d, J=8.4 Hz, 2H), 6.89-6.86 (m, 1H), 6.74 (s, 1H), 3.88 (s, 3H), 3.28-3.25 (m, 2H), 2.69-2.66 (m, 2H). LCMS (ES, m/z)⁺: 337 [M+H]⁺.

### Example 13

### Synthesis of 2-phenyl-N-[(trans)-3-[(cyanoamino)methyl]cyclobutyl]-1,3-thiazole-5-carboxamide (16)

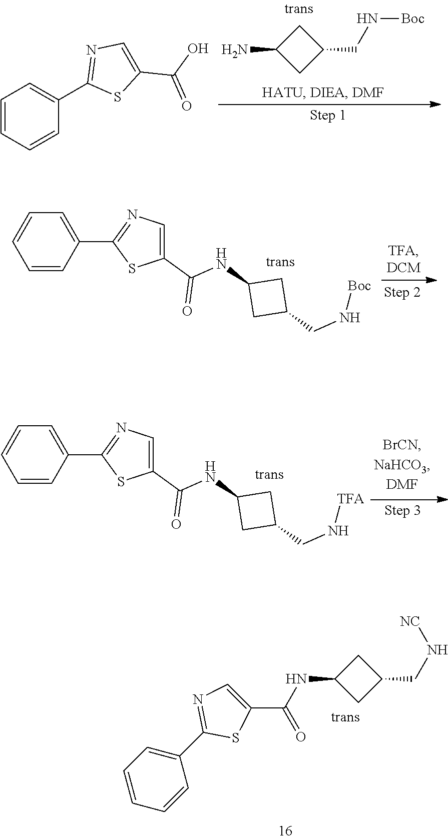

### Step 1. Tert-butyl N-[[(trans)-3-(2-phenyl-1, 3-thiazole-5-amido) cyclobutyl]methyl] carbamate

A mixture of 2-phenyl-1,3-thiazole-5-carboxylic acid (184 mg, 0.88 mmol), tert-butyl N-[[(trans)-3-aminocyclobutyl]methyl] carbamate (150 mg, 0.73 mmol), HATU (335 mg, 0.88 mmol) and DIEA (0.40 mL, 2.82 mmol) in DMF (3 mL) was stirred for 1 hour at 25° C. The mixture was diluted with water (20 mL) and extracted with EtOAc (3×15 mL). The combined organic layers were washed with brine (3×5 mL) and dried over anhydrous Na₂SO₄. After filtration, the filtrate was concentrated under reduced pressure. The residue was purified by silica gel column chromatography, eluted with CH₂C₂/MeOH (10:1) to afford tert-butyl N-[[(trans)-3-(2-phenyl-1,3-thiazole-5-amido)cyclobutyl]methyl]carbamate as a white solid (250 mg, 74%). LCMS (ES, m/z)⁺: 388 [M+H]⁺.

### Step 2. TFA salt of 2-phenyl-N-[(trans)-3-(aminomethyl) cyclobutyl]-1, 3-thiazole-5-carboxamide

A mixture of tert-butyl N-[[(trans)-3-(2-phenyl-1, 3-thiazole-5-amido) cyclobutyl]methyl] carbamate (150 mg, 0.39 mmol) and TFA (1.50 mL) in DCM (5 mL) was stirred for 30 minutes at 25° C. The resulting mixture was concentrated under vacuum to afford TFA salt of 2-phenyl-N-[(1r, 3r)-3-(aminomethyl) cyclobutyl]-1, 3-thiazole-5-carboxamide as a yellow oil (150 mg, crude). LCMS (ES, m/z)⁺: 288 [M−TFA+H]⁺.

### Step 3. 2-phenyl-N-[(trans)-3-[(cyanoamino)methyl]cyclobutyl]-1,3-thiazole-5-carboxamide (16)

A mixture of TFA salt of 2-phenyl-N-[(trans)-3-(aminomethyl) cyclobutyl]-1, 3-thiazole-5-carboxamide (150 mg, 0.37 mmol), NaHCO₃ (368 mg, 4.38 mmol) and BrCN (46.4 mg, 0.44 mmol) in DMF (3 mL) was stirred for 1 hour at 25° C. The mixture was diluted with water/ice (20 mL) and extracted with EtOAc (3×10 mL). The combined organic layers were washed with brine (3×5 mL) and dried over anhydrous Na₂SO₄. After filtration, the filtrate was concentrated under reduced pressure.

The residue was purified by Prep-HPLC with the following condition: Column: XBridge Shield RP18 OBD Column, 5 μm, 19×150 mm; Mobile Phase, water (containing 0.05% ammonia) and CH₃CN (25% to 45% over 7 minutes); Detector: UV 254/220 nm. The product fractions were lyophilized to afford 2-phenyl-N-[(trans)-3-[(cyanoamino)methyl]cyclobutyl]-1,3-thiazole-5-carboxamide (16) as a white solid (22.5 mg, 20%).

16 ¹H-NMR (DMSO-d6, 400 MHz) δ (ppm): 8.93 (d, J=6.8 Hz, 1H), 8.48 (s, 1H), 7.99 (br s, 2H), 7.54 (br s, 3H), 6.84 (br s, 1H), 4.48-4.46 (m, 1H), 3.10-3.07 (m, 2H), 2.41 (br s, 1H), 2.23-2.19 (m, 2H), 2.13 (br s, 2H). LCMS (ES, m/z)⁺: 313 [M+H]⁺.

### Example 14

### Synthesis of (2R)-3-(cyanoamino)-2-methyl-N-[3-[4-(I-methylpyrazol-4-yl)phenyl]1,2-oxazol-5-yl]propanamide (18)

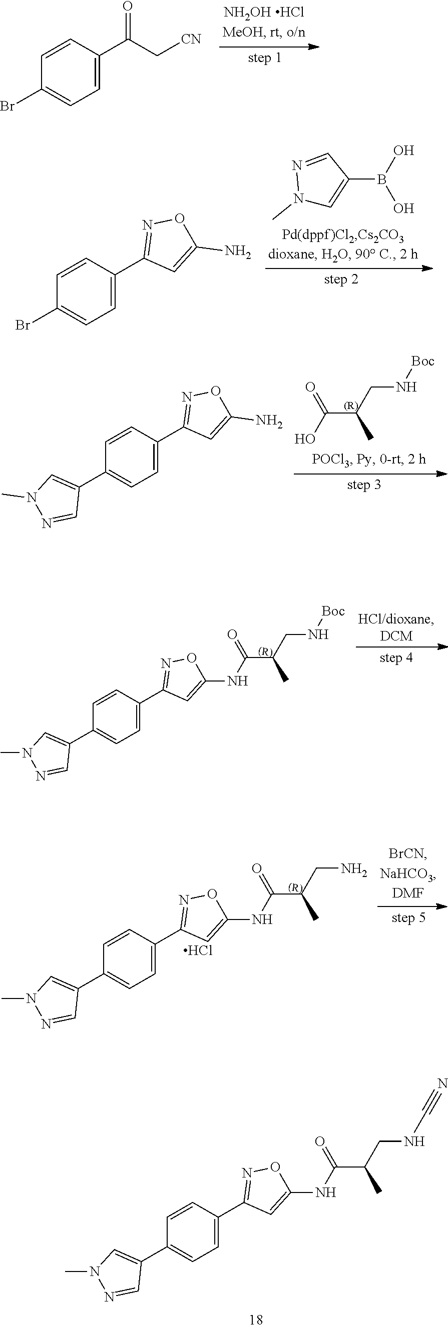

### Step 1. 3-(4-bromophenyl)-1,2-oxazol-5-amine

A mixture of 3-(4-bromophenyl)-3-oxopropanenitrile (20.0 g, 87.4 mmol), NH₂NH₂.HCl (8.98. g, 131 mmol) and NaOAc (10.8 g, 131 mmol) in MeOH (100 mL) was stirred overnight at 24° C. The mixture was diluted with water (300 mL) and extracted with EtOAc (3×300 mL). The combined organic layers were dried over anhydrous Na₂SO₄. After filtration, the filtrate was concentrated under reduced pressure to afford 3-(4-bromophenyl)-1,2-oxazol-5-amine (16.0 g, 73%) as a yellow solid. LCMS (ES, m/z)⁺: 239, 241 [M+H]⁺.

### Step 2. 3-[4-(1-methylpyrazol-4-yl)phenyl]-1,2-oxazol-5-amine

To a stirred mixture of 3-(4-bromophenyl)-1,2-oxazol-5-amine (10.0 g, 41.8 mmol) in dioxane (200 mL) was added 1-methylpyrazol-4-ylboronic acid (10.0 g, 79.4 mmol), Pd(dppf)Cl₂.CH₂Cl₂ (3.40 g, 4.16 mmol), Cs2CO3 (40.0 g, 123 mmol) and H₂O (60 mL). The resulting mixture was stirred for 6 hours at 90° C. under a nitrogen atmosphere. The mixture was cooled to room temperature and concentrated under reduced pressure to remove dioxane. The precipitated solids were collected by filtration and washed with water (300 mL), MeOH (3×20 mL) and ethyl acetate (3×100 mL). This resulted in 3-[4-(1-methylpyrazol-4-yl)phenyl]-1,2-oxazol-5-amine (6.60 g, 53%) as a brown solid. LCMS (ES, m/z)⁺: 241 [M+H]⁺.

### Step 3. tert-butyl N-[(2R)-2-methyl-2-([3-[4-(1-methylpyrazol-4-yl)phenyl]-1,2-oxazol-5-yl]carbamoyl)ethyl]carbamate

To a stirred mixture of 3-[4-(1-methylpyrazol-4-yl)phenyl]-1,2-oxazol-5-amine (100 mg, 0.42 mmol) and (2R)-3-[(tert-butoxycarbonyl)amino]-2-methylpropanoic acid (93 mg, 0.46 mmol) in pyridine (3 mL) was added POCl₃ (96 mg, 0.63 mmol) dropwise at 0° C. under a nitrogen atmosphere. The resulting mixture was stirred for 2 hours at 25° C. under nitrogen atmosphere. The mixture was poured into water/ice (20 mL) and extracted with CH₂Cl₂ (3×20 mL). The combined organic layers were washed with brine (20 mL), dried over anhydrous Na₂SO₄ and concentrated under reduced pressure. The residue was purified by silica gel column chromatography, eluted with petroleum ether/EtOAc (1:1), to afford tert-butyl N-[(2R)-2-methyl-2-([3-[4-(1-methylpyrazol-4-yl)phenyl]-1,2-oxazol-5-yl]carbamoyl)ethyl]carbamate (80 mg, 43%) as a light yellow solid. LCMS (ES, m/z)⁺: 426 [M+H]⁺.

### Step 4. (2R)-3-amino-2-methyl-N-[3-[4-(1-methylpyrazol-4-yl)phenyl]-1,2-oxazol-5-yl]propanamide hydrochloride

To a stirred mixture of tert-butyl N-[(2R)-2-methyl-2-([3-[4-(1-methylpyrazol-4-yl)phenyl]-1,2-oxazol-5-yl]carbamoyl)ethyl]carbamate (75 mg, 0.18 mmol) in DCM (6 mL) was added HCl in 1,4-dioxane (3 mL, 4M) dropwise at 0° C. under a nitrogen atmosphere. The resulting mixture was stirred for 2 hours at 0° C. under a nitrogen atmosphere. The resulting mixture was concentrated under reduced pressure to afford (2R)-3-amino-2-methyl-N-[3-[4-(1-methylpyrazol-4-yl)phenyl]-1,2-oxazol-5-yl]propanamide hydrochloride (50 mg, 74%) as a light yellow solid. LCMS (ES, m/z)⁺: 326 [M−HCl⁺H]⁺.

### Step 5. (2R)-3-(cyanoamino)-2-methyl-N-[3-[4-(1-methylpyrazol-4-yl)phenyl]-1,2-oxazol-5-yl]propanamide (18)

To a stirred mixture of (2R)-3-amino-2-methyl-N-[3-[4-(1-methylpyrazol-4-yl)phenyl]-1,2-oxazol-5-yl]propanamide hydrochloride (75 mg, 0.21 mmol) in DMF (2 mL) was added NaHCO₃ (98 mg, 1.17 mmol) and cyanogen bromide (23 mg, 0.22 mmol) at 0° C. under a nitrogen atmosphere. The resulting mixture was stirred for 2 hours at 25° C. under nitrogen atmosphere. The mixture was poured into water/ice (20 mL) and extracted with CH₂Cl₂ (3×20 mL). The combined organic layers were washed with brine (10 mL), dried over anhydrous Na₂SO₄ and concentrated under reduced pressure.

The crude product was purified by Prep-HPLC with the following conditions: Column, XBridge Shield RP18 OBD Column, 5 μm, 19×150 mm; mobile phase, H₂O and ACN (25% up to 40% in 7 minutes); Detector, UV254/220 nm. The collected fraction was lyophilized to afford (2R)-3-(cyanoamino)-2-methyl-N-[3-[4-(1-methylpyrazol-4-yl)phenyl]-1,2-oxazol-5-yl]propanamide (18) (10.3 mg, 13%) as a white solid.

18 ¹H NMR (400 MHz, DMSO-d6) δ (ppm): 11.86 (s, 1H), 8.24 (s, 1H), 7.95 (s, 1H), 7.83 (d, J=8.0 Hz, 2H), 7.70 (d, J=8.4 Hz, 2H), 6.96-6.93 (m, 1H), 6.76 (s, 1H), 3.88 (s, 3H), 3.32-3.21 (m, 1H), 3.06-3.00 (m, 1H), 2.85-2.80 (m, 1H), 1.15 (d, J=6.8 Hz, 3H). LCMS (ES, m/z)⁺: 351 [M+H]⁺.

### Example 15

### Synthesis of (2S)-3-(cyanoamino)-2-methyl-N-[3-[4-(I-methylpyrazol-4-yl)phenyl]1,2-oxazol-5-yl]propanamide (19)

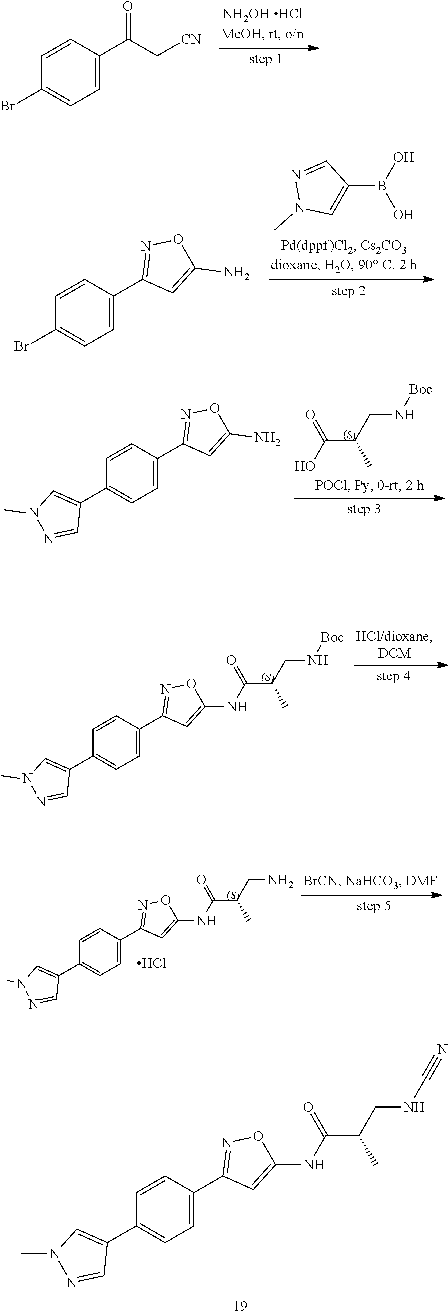

### Step 1. 3-(4-bromophenyl)-1,2-oxazol-5-amine

A mixture of 3-(4-bromophenyl)-3-oxopropanenitrile (20.0 g, 87.4 mmol), NH₂NH₂.HCl (8.98. g, 131 mmol) and NaOAc (10.8 g, 131 mmol) in MeOH (100 mL) was stirred overnight at 24° C. The mixture was diluted with water (300 mL) and extracted with EtOAc (3×300 mL). The combined organic layers were dried over anhydrous Na₂SO₄. After filtration, the filtrate was concentrated under reduced pressure to afford 3-(4-bromophenyl)-1,2-oxazol-5-amine (16.0 g, 73%) as a yellow solid. LCMS (ES, m/z)⁺: 239, 241 [M+H]⁺.

### Step 2. 3-[4-(1-methylpyrazol-4-yl)phenyl]-1,2-oxazol-5-amine

To a stirred mixture of 3-(4-bromophenyl)-1,2-oxazol-5-amine (10.0 g, 41.8 mmol) in dioxane (200 mL) were added 1-methylpyrazol-4-ylboronic acid (10.0 g, 79.4 mmol), Pd(dppf)Cl₂.CH₂Cl₂ (3.40 g, 4.16 mmol), Cs₂CO₃ (40.0 g, 123 mmol) and H₂O (60 mL). The resulting mixture was stirred for 6 hours at 90° C. under a nitrogen atmosphere. The mixture was cooled to room temperature and concentrated under reduced pressure to remove dioxane. The precipitated solids were collected by filtration and washed with water (300 mL), MeOH (3×20 mL) and ethyl acetate (3×100 mL). This resulted in 3-[4-(1-methylpyrazol-4-yl)phenyl]-1,2-oxazol-5-amine (6.60 g, 53%) as a brown solid. LCMS (ES, m/z)⁺: 241 [M+H]⁺.

### Step 3. tert-butyl N-[(2S)-2-methyl-2-([3-[4-(1-methylpyrazol-4-yl)phenyl]-1,2-oxazol-5-yl]carbamoyl)ethyl]carbamate

To a stirred mixture of 3-[4-(1-methylpyrazol-4-yl)phenyl]-1,2-oxazol-5-amine (120 mg, 0.50 mmol) and (2S)-3-[(tert-butoxycarbonyl)amino]-2-methylpropanoic acid (100 mg, 0.49 mmol) in pyridine (3 mL) was added POCl₃ (110 mg, 0.72 mmol) dropwise at 0° C. under a nitrogen atmosphere. The resulting mixture was stirred for 3 hours at 25° C. The mixture was poured into water/ice (20 mL) and extracted with CH2Cl2 (3×20 mL). The combined organic layers were washed with brine (20 mL), dried over anhydrous Na₂SO₄ and concentrated under reduced pressure.

The residue was purified by reverse flash chromatography with the following conditions: column, C18 silica gel; mobile phase, water with TFA (0.05%) and ACN (0% to 100% gradient in 30 minutes); detector, UV 254/220 nm. The collected fraction concentrated under reduced pressure to afford tert-butyl N-[(2S)-2-methyl-2-([3-[4-(1-methylpyrazol-4-yl)phenyl]-1,2-oxazol-5-yl]carbamoyl)ethyl]carbamate (108 mg, 48%) as a light yellow solid. LCMS (ES, m/z)⁺: 426 [M+H]⁺.

### Step 4. (2S)-3-amino-2-methyl-N-[3-[4-(1-methylpyrazol-4-yl)phenyl]-1,2-oxazol-5-yl]propanamide hydrochloride

To a stirred mixture of tert-butyl N-[(2S)-2-methyl-2-([3-[4-(1-methylpyrazol-4-yl)phenyl]-1,2-oxazol-5-yl]carbamoyl)ethyl]carbamate (100 mg, 0.24 mmol) in DCM (5 mL) was added HCl in 1,4-dioxane (2.5 mL, 4M) dropwise at 0° C. under nitrogen atmosphere. The resulting mixture was stirred for 2 hours at 25° C. under nitrogen atmosphere. The resulting mixture was concentrated under reduced pressure to afford (2S)-3-amino-2-methyl-N-[3-[4-(1-methylpyrazol-4-yl)phenyl]-1,2-oxazol-5-yl]propanamide hydrochloride (85 mg, 97%) as a light grey solid. LCMS (ES, m/z)⁺: 326 [M−HCl⁺H]⁺.

### Step 5. (2S)-3-(cyanoamino)-2-methyl-N-[3-[4-(1-methylpyrazol-4-yl)phenyl]-1,2-oxazol-5-yl]propanamide (19)

To a stirred mixture of (2S)-3-amino-2-methyl-N-[3-[4-(1-methylpyrazol-4-yl)phenyl]-1,2-oxazol-5-yl]propanamide hydrochloride (82 mg, 0.23 mmol) in DMF (2 mL) were added NaHCO₃ (95 mg, 1.13 mmol) and cyanogen bromide (24 mg, 0.23 mmol) at 0° C. under nitrogen atmosphere. The resulting mixture was stirred for 5 hours at 25° C. under nitrogen atmosphere.

The crude product was purified by Prep-HPLC with the following conditions: Column, XBridge Shield RP18 OBD Column, 5 μm, 19×150 mm; mobile phase, water and ACN (27% up to 38% in 7 minutes); Detector, UV220/254 nm. The collected fraction was lyophilized to afford (2S)-3-(cyanoamino)-2-methyl-N-[3-[4-(1-methylpyrazol-4-yl)phenyl]-1,2-oxazol-5-yl]propanamide (19) (22.4 mg, 27%) as a white solid.

19 ¹H NMR (400 MHz, DMSO-d6) δ (ppm): 11.86 (s, 1H), 8.24 (s, 1H), 7.95 (s, 1H), 7.83 (d, J=8.0 Hz, 2H), 7.70 (d, J=8.0 Hz, 2H), 6.96-6.93 (m, 1H), 6.76 (s, 1H), 3.88 (s, 3H), 3.33-3.21 (m, 1H), 3.06-3.00 (m, 1H), 2.85-2.80 (m, 1H), 1.14 (d, J=6.8 Hz, 3H). LCMS (ES, m/z)⁺: 351 [M+H]⁺.

### Example 16

### Synthesis of N-methyl-2-phenyl-N-[(trans)-3-[(cyanoamino)methyl]cyclobutyl]-1, 3-thiazole-5-carboxamide (20)

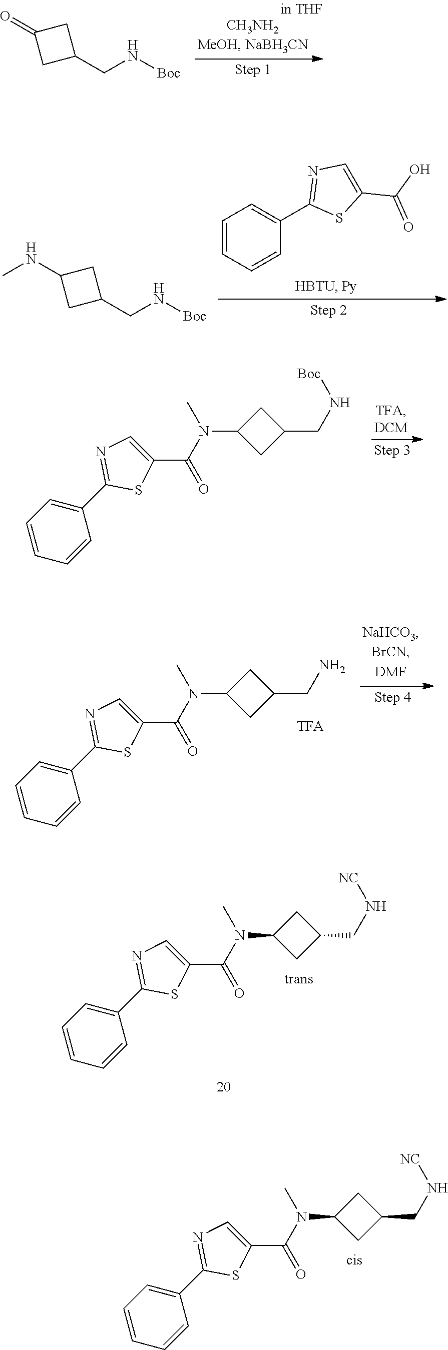

### Step 1. Tert-butyl N-[[3-(methylamino)cyclobutyl]methyl]carbamate

To a stirred solution of tert-butyl N-[(3-oxocyclobutyl)methyl]carbamate (200 mg, 0.98 mmol), methanamine (3.00 mL, 1 M in THF) in MeOH (2 mL) and NaBH₃CN (130 mg, 2.03 mmol) in CH₃COOH (0.2 mL) was added in portions at 0° C. The resulting mixture was stirred for 4 hours at 25° C. The mixture was diluted with water (20 mL) and extracted with ethyl acetate (3×50 mL). The combined organic layers were washed with brine (3×50 mL) and dried over anhydrous sodium sulfate. After filtration, the filtrate was concentrated under reduced pressure. The residue was purified by silica gel chromatography (eluting with 10:1 dichloromethane/methanol) to afford tert-butyl N-[[3-(methylamino)cyclobutyl]methyl]carbamate as yellow oil (66 mg, 28%). LCMS (ES, m/z)⁺: 215 [M+H]⁺.

### Step 2. Tert-butyl N-[[3-(N-methyl 2-phenyl-1,3-thiazole-5-amido)cyclobutyl]methyl]carbamate

A mixture of tert-butyl N-[[3-(methylamino)cyclobutyl]methyl]carbamate (57 mg, 0.24 mmol), 2-phenyl-1,3-thiazole-5-carboxylic acid (50 mg, 0.24 mmol) and HBTU (139 mg, 0.36 mmol) in pyridine (2 mL) was stirred for 2 hours at 100° C. The mixture was cooled to room temperature, diluted with water (10 mL) and extracted with ethyl acetate (3×50 mL). The combined organic layers were washed with brine (50 mL) and dried over anhydrous sodium sulfate. After filtration, the filtrate was concentrated under reduced pressure. The residue was purified by silica gel chromatography (eluting with 1:1 petroleum ether/ethyl acetate) to afford tert-butyl N-[[3-(N-methyl 2-phenyl-1,3-thiazole-5-amido)cyclobutyl]methyl]carbamate as yellow oil (30 mg, 27%). LCMS (ES, m/z)⁺: 402 [M+H]⁺.

### Step 3. TFA salt of N-[3-(aminomethyl)cyclobutyl]-N-methyl-2-phenyl-1,3-thiazole-5-carboxamide

A solution of tert-butyl N-[[3-(N-methyl2-phenyl-1,3-thiazole-5-amido)cyclobutyl]methyl]carbamate (30 mg, 0.07 mmol) in TFA (1 mL) and DCM (2 mL) was stirred for 2 hours at 25° C. The resulting mixture was concentrated under vacuum to afford the TFA salt of N-[3-(aminomethyl)cyclobutyl]-N-methyl-2-phenyl-1,3-thiazole-5-carboxamide (30 mg, crude) as a yellow oil. LCMS (ES, m/z)⁺: 302 [M−TFA+H]⁺.

### Step 4. N-methyl-2-phenyl-N-[(trans)-3-[(cyanoamino)methyl]cyclobutyl]-1,3-thiazole-5-carboxamide (20) and N-methyl-2-phenyl-N-[(cis)-3-[(cyanoamino)methyl]cyclobutyl]-1,3-thiazole-5-carboxamide

To a stirred mixture of TFA salt of N-[3-(aminomethyl)cyclobutyl]-N-methyl-2-phenyl-1,3-thiazole-5-carboxamide (30 mg, 0.07 mmol) and NaHCO₃ (28 mg, 0.33 mmol) in DMF (0.5 mL), BrCN (7 mg, 0.06 mmol) was added at 0° C. The resulting mixture was stirred for 1 hour at 25° C. The mixture was diluted with water (10 mL) and extracted with ethyl acetate (3×10 mL). The combined organic layers were washed with brine (20 mL) and dried over anhydrous sodium sulfate. After filtration, the filtrate was concentrated under reduced pressure.

The residue was purified by Prep-HPLC with the following conditions: Column: XBridge RP18 OBD Column, 5 μm, 30×150 mm; Mobile Phase, A: water (containing 0.05% ammonium bicarbonate) and B: ACN (32% to 43% in 7 minutes); Detector: UV 254 nm. The product fractions were lyophilized to afford mixture of trans- and cis-product as a white solid (15 mg).

The mixture of trans- and cis-isomers was separated by Chiral-Prep-HPLC (Column, CHIRALPAK IC, 5 μm, 2×25 cm; Mobile phase, A: methyl tert-butyl ester (containing 0.1% diethylamine) and B: ethanol (hold 30% to 30% in 12 minutes); Flow rate: 20 mL/minute; Detector: 254 and 220 nm; RT1: 8.576 minutes; RT2: 9.916 minutes). The product fractions were lyophilized to afford N-methyl-2-phenyl-N-[(trans)-3-[(cyanoamino)methyl]cyclobutyl]-1,3-thiazole-5-carboxamide (20) (first eluting isomer, RT1: 8.576) as a white solid (3 mg, 48%) and N-methyl-2-phenyl-N-[(1r,3r)-3-[(cyanoamino)methyl]cyclobutyl]-1,3-thiazole-5-carboxamide (second eluting isomer, RT2: 9.916) as a white solid (8 mg, 42%).

20 ¹H-NMR (DMSO-d6, 400 MHz) δ (ppm): 8.16 (s, 1H), 8.01-7.98 (m, 2H), 7.56-7.53 (m, 3H), 6.84 (s, 1H), 4.91-4.90 (m, 1H), 3.13-3.07 (m, 5H), 2.52-2.42 (m, 2H), 2.36-2.34 (m, 1H), 2.06-2.01 (m, 2H). LCMS (ES, m/z)⁺: 327 [M+H]⁺.

Cis-isomer ¹H-NMR (DMSO-d6, 400 MHz) δ (ppm): 8.19 (s, 1H), 8.01-7.99 (m, 2H), 7.56-7.53 (m, 3H), 6.78 (s, 1H), 4.63-4.62 (m, 1H), 3.09 (br s, 3H), 3.03-3.00 (m, 2H), 2.28-2.25 (m, 2H), 2.13-2.23 (m, 1H), 2.05-2.00 (m, 2H). LCMS (ES, m/z)⁺: 327 [M+H]⁺.

### Example 17

### Synthesis of Cyano({[(2S)-4-[2-(2,4-dichlorophenyl)-1, 3-thiazole-4-carbonyl]morpholin-2-yl]methyl})amine (24)

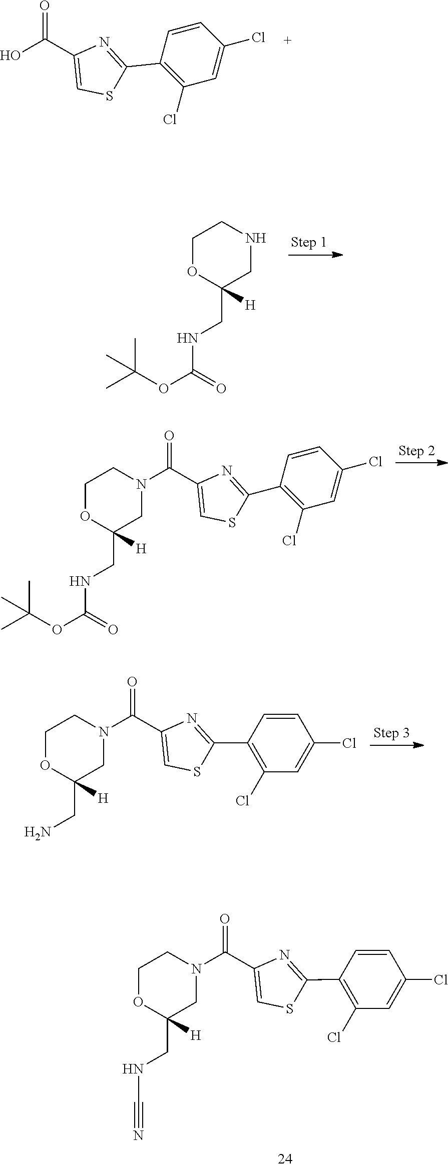 [SMILES: O=C(O)c1csc(-c2ccc(Cl)cc2Cl)n1 ; CC(C)(C)OC(=O)NC[C@H]1CNCCO1 ; CC(C)(C)OC(=O)NC[C@H]1CN(C(=O)c2csc(-c3ccc(Cl)cc3Cl)n2)CCO1 ; NC[C@H]1CN(C(=O)c2csc(-c3ccc(Cl)cc3Cl)n2)CCO1 ; N#CNC[C@H]1CN(C(=O)c2csc(-c3ccc(Cl)cc3Cl)n2)CCO1]

### Step 1. (tert-Butyl (S)-((4-(2-(2,4-dichlorophenyl)thiazole-4-carbonyl)morpholin-2-yl)methyl)carbamate

In a 2 mL reaction vial, 2-(2,4-dichlorophenyl)thiazole-4-carboxylic acid (0.2 M 1,4-dioxane, 0.200 mL, 0.040 mmol) and 2-(7-aza-1H-benzotriazole-1-yl)-1,1,3,3-tetramethyluronium hexafluorophosphate (0.2 M acetonitrile, 0.220 mL, 0.044 mmol) were combined with neat DIEA (0.035 mL, 0.200 mmol). The reaction vial was sealed and agitated at ambient temperature for 15 minutes. The vial was charged with tert-butyl (R)-(morpholin-2-ylmethyl)carbamate (0.2 M 9:1 acetonitrile/DIEA, 0.220 mL, 0.044 mmol) and agitated at 50° C. for 2-24 hours. The reaction was concentrated under a stream of nitrogen, and the residue was partitioned between ethyl acetate (0.5 mL) and 1:1 brine/water (0.5 mL). The organic layer was separated, and the residual aqueous layer was extracted with fresh ethyl acetate (0.5 mL). The organic layer was separated and combined with the first extract. The extracts were dried under vacuum at 50° C. to provide (tert-butyl (S)-((4-(2-(2,4-dichlorophenyl)thiazole-4-carbonyl)morpholin-2-yl)methyl)carbamate.

### Step 2. (S)-(2-(Aminomethyl)morpholino)(2-(2,4-dichlorophenyl)thiazol-4-yl) methanone

(tert-Butyl (S)-((4-(2-(2,4-dichlorophenyl)thiazole-4-carbonyl)morpholin-2-yl)methyl)carbamate (Step 1) was dissolved in 1,4-dioxane/methanol (1:1 v/v, 0.200 mL) and treated with hydrochloric acid (4.0 M 1,4-dioxane, 0.075 mL, 0.3 mmol). The vial was sealed and agitated at 50° C. for 2 hours. The reaction was cooled and concentrated at 50° C. to provide (S)-(2-(aminomethyl)morpholino)(2-(2,4-dichlorophenyl)thiazol-4-yl)methanone, hydrochloric acid salt which was used without further purification.

### Step 3. Cyano({[(2S)-4-[2-(2,4-dichlorophenyl)-1,3-thiazole-4-carbonyl]morpholin-2-yl]methyl})amine

(S)-(2-(Aminomethyl)morpholino)(2-(2,4-dichlorophenyl)thiazol-4-yl)methanone, hydrochloric acid salt (Step 2) was suspended in acetonitrile (0.200 mL). The mixture was made homogeneous with the addition of DIEA (0.070 mL, 0.400 mmol) before cyanogen bromide (0.2 M acetonitrile, 0.200 mL, 0.040 mmol) was added. The vial was sealed and agitated at ambient temperature for 4 hours. The reaction mixture was diluted with DMSO (0.300 mL) and immediately purified by mass triggered preparative HPLC (neutral method) to provide the title compound (3.2 mg, 20%). LCMS (ESI)⁺: m/z 397.04 [M+H]⁺.

### Example 18

### Synthesis of (2R)-3-(cyanoamino)-N-[2-(3-methoxyphenyl)-1,3-thiazol-5-yl]-2-methylpropanamide (43)

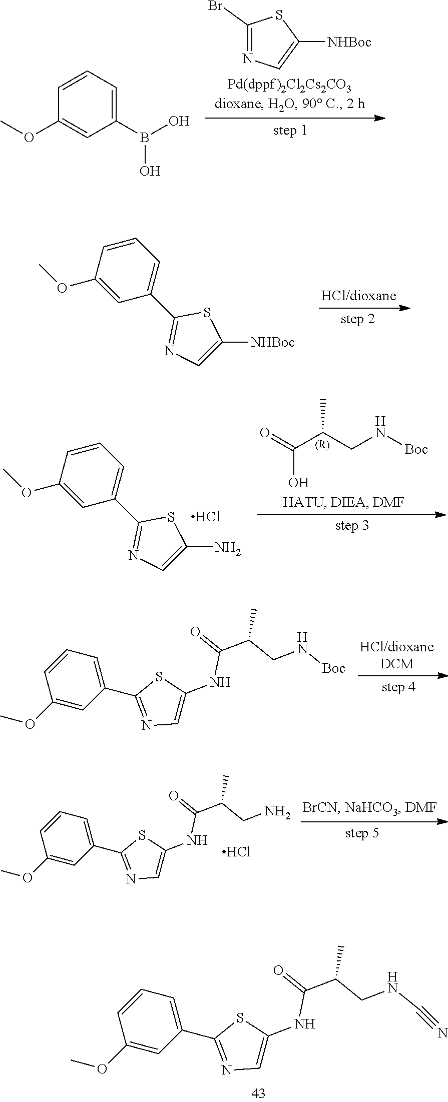

### Step 1. tert-butyl N-[2-(3-methoxyphenyl)-1,3-thiazol-5-yl]carbamate

To a stirred solution of tert-butyl N-(2-bromo-1,3-thiazol-5-yl)carbamate (6.00 g, 21.0 mmol), 3-methoxyphenylboronic acid (4.80 g, 31.5 mmol) and Pd(dppf)Cl₂ (1.03 g, 1.40 mmol) in dioxane (60 mL) was added K₃PO₄ (13.4 g, 63.1 mmol) in H₂O (20 mL) under a nitrogen atmosphere. The resulting mixture was stirred for 1 hour at 90° C. under nitrogen atmosphere. The mixture was cooled to room temperature. The resulting mixture was diluted with water (500 mL) and extracted with EtOAc (3×500 mL). The combined organic layers were dried over anhydrous Na₂SO₄ and concentrated under reduced pressure. The residue was purified by silica gel chromatography (eluting with 1:1 ethyl acetate/petroleum ether) to afford tert-butyl N-[2-(3-methoxyphenyl)-1,3-thiazol-5-yl]carbamate as a yellow solid (4.80 g, 71%). LCMS (ES, m/z)⁺: 307 [M+H]⁺.

### Step 2. 2-(3-methoxyphenyl)-1,3-thiazol-5-amine hydrochloride

To a stirred solution of tert-butyl N-[2-(3-methoxyphenyl)-1,3-thiazol-5-yl]carbamate (3.80 g, 11.9 mmol) in MeOH (10 mL) was added HCl in 1,4-dioxane (20 mL). The resulting mixture was stirred for 15 hours at 24° C. The mixture was concentrated under vacuum to afford 2-(3-methoxyphenyl)-1,3-thiazol-5-amine hydrochloride as a yellow solid (2.80 g, 93%). LCMS (ES, m/z)⁺: 207 [M−HCl+H]⁺.

### Step 3. tert-butyl N-[(2R)-2-[[2-(3-methoxyphenyl)-1,3-thiazol-5-yl]carbamoyl]-2-ethylethyl]carbamate

To a stirred mixture of (2R)-3-[(tert-butoxycarbonyl)amino]-2-methylpropanoic acid (122 mg, 0.60 mmol) and HATU (281 mg, 0.74 mmol) in DMF (3 mL) were added DIEA (244 uL, 1.48 mmol) and 2-(3-methoxyphenyl)-1,3-thiazol-5-amine hydrochloride (100 mg, 0.42 mmol) at 0° C. The resulting mixture was stirred for 16 hours at 25° C. The mixture was diluted with water (50 mL) and extracted with EtOAc (3×50 mL). The combined organic layers were dried over anhydrous Na₂SO₄ and concentrated under reduced pressure. The crude product was purified by silica gel column chromatography, eluted with petroleum ether/EtOAc (1:1), to afford tert-butyl N-[(2R)-2-[[2-(3-methoxyphenyl)-1,3-thiazol-5-yl]carbamoyl]-2-methylethyl]carbamate (97 mg, 59%) as a yellow oil. LCMS (ES, m/z)⁺: 392 [M+H]⁺.

### Step 4. (2R)-3-amino-N-[2-(3-methoxyphenyl)-1,3-thiazol-5-yl]-2-methyl propanamide hydrochloride

To a stirred solution of tert-butyl N-[(2R)-2-[[2-(3-methoxyphenyl)-1,3-thiazol-5-yl]carbamoyl]-2-methylethyl]carbamate (90 mg, 0.23 mmol) in DCM (6 mL) was added HCl in 1,4-dioxane (3 mL, 4M) dropwise at 0° C. under a nitrogen atmosphere. The resulting mixture was stirred for 2 hours at 25° C. The mixture was concentrated under reduced pressure to afford (2R)-3-amino-N-[2-(3-methoxyphenyl)-1,3-thiazol-5-yl]-2-methylpropanamide hydrochloride (70 mg, 88%) as a light yellow solid. LCMS (ES, m/z)⁺: 292 [M−HCl⁺H]⁺.

### Step 5. (2R)-3-(cyanoamino)-N-[2-(3-methoxyphenyl)-1,3-thiazol-5-yl]-2-methylpropanamide (43)

To a stirred solution of (2R)-3-amino-N-[2-(3-methoxyphenyl)-1,3-thiazol-5-yl]-2-methylpropanamide hydrochloride (70 mg, 0.21 mmol) in DMF (2 mL) was added NaHCO₃ (104 mg, 1.24 mmol) and cyanogen bromide (26 mg, 0.25 mmol) dropwise at 0° C. The resulting mixture was stirred for 5 hours at 25° C. under a nitrogen atmosphere.

The crude product was purified by Prep-HPLC with the following conditions: Column, XBridge Shield RP18 OBD Column, 5 μm, 19×150 mm; mobile phase, H₂O and ACN (30% up to 40% in 7 minutes); Detector, UV220/254 nm. The collected fraction was lyophilized to afford (2R)-3-(cyanoamino)-N-[2-(3-methoxyphenyl)-1,3-thiazol-5-yl]-2-methylpropanamide (43) (33.5 mg, 42%) as a light yellow solid.

43 ₁H NMR (400 MHz, DMSO-d6) δ (ppm): 11.62 (s, 1H), 7.61 (s, 1H), 7.44-7.37 (m, 3H), 7.01-6.95 (m, 2H), 3.83 (s, 3H), 3.26-3.19 (m, 1H), 3.05-2.99 (m, 1H), 2.80-2.75 (m, 1H), 1.14 (d, J=6.8 Hz, 3H). LCMS (ES, m/z)⁺: 317 [M+H]⁺.

### Example 19

### Synthesis of {[(2r,4s)-6-[3-(pyridin-2-yl)-1, 2-oxazole-5-carbonyl]-6-azaspiro[3.4]octan-2-yl]amino}formonitrile (45)

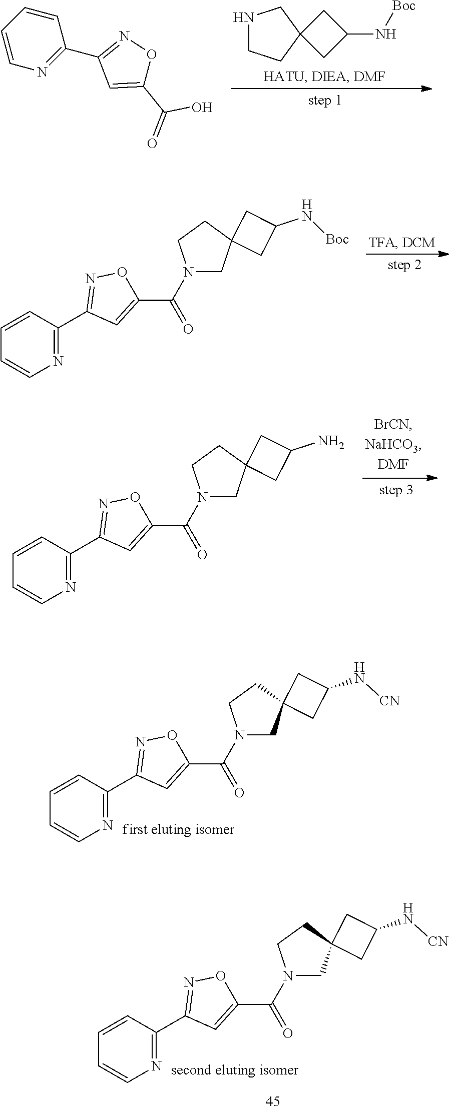 [SMILES: O=C(O)c1cc(-c2ccccn2)no1 ; CC(C)(C)OC(=O)NC1CC2(CCNC2)C1 ; CC(C)(C)OC(=O)NC1CC2(CCN(C(=O)c3cc(-c4ccccn4)no3)C2)C1 ; NC1CC2(CCN(C(=O)c3cc(-c4ccccn4)no3)C2)C1 ; N#CN[C@H]1C[C@@]2(CCN(C(=O)c3cc(-c4ccccn4)no3)C2)C1 ; N#CN[C@H]1C[C@]2(CCN(C(=O)c3cc(-c4ccccn4)no3)C2)C1]

### Step 1. Tert-butyl N-[6-[3-(pyridin-2-yl)-1,2-oxazole-5-carbonyl]-6-azaspiro[3.4]octan-2-yl] carbamate

To a stirred mixture of 3-(pyridin-2-yl)-1,2-oxazole-5-carboxylic acid (130 mg, 0.67 mmol) in DMF (3 mL) was added HATU (321 mg, 0.83 mmol), tert-butyl N-[6-azaspiro[3.4]octan-2-yl]carbamate (149 mg, 0.65 mmol) and DIEA (286 uL, 1.70 mmol) dropwise at 0° C. The resulting mixture was stirred for 2 hours at room temperature. The reaction mixture was diluted with water (30 mL) and extracted with DCM (3×20 mL). The combined organic layers were washed with brine (2×30 mL) and dried over anhydrous Na₂SO₄. After filtration, the filtrate was concentrated under reduced pressure. The residue was purified by reverse flash chromatography with the following conditions (Column: C18 silica gel, 40 g; Mobile phase: water (containing 0.05% NH₄HCO₃) and ACN (5% to 30% in 20 min); Detector: UV 254/220 nm), affording tert-butyl N-[6-[3-(pyridin-2-yl)-1, 2-oxazole-5-carbonyl]-6-azaspiro [3.4] octan-2-yl]carbamate (210 mg, 78%) as a white solid. LCMS (ES, m/z)⁺: 399 [M+H]⁺.

### Step 2. 6-[3-(pyridin-2-yl)-1, 2-oxazole-5-carbonyl]-6-azaspiro [3.4]octan-2-amine

To a stirred mixture of tert-butyl N-[6-[3-(pyridin-2-yl)-1,2-oxazole-5-carbonyl]-6-azaspiro[3.4]octan-2-yl] carbamate (210 mg, 0.52 mmol) in DCM (6 mL) was added TFA (3 mL) dropwise at 0° C. The resulting mixture was stirred for 2 hours at 24° C. The mixture was concentrated under vacuum. The residue was dissolved in water (10 mL), the resulting mixture was basified to pH 8 with NaHCO₃ (sat., aq.) and extracted with DCM (3×10 mL). The combined organic layers were dried over anhydrous Na₂SO₄ and concentrated under reduced pressure to afford 6-[3-(pyridin-2-yl)-1,2-oxazole-5-carbonyl]-6-azaspiro [3.4]octan-2-amine (150 mg, 92%) as a white solid. LCMS (ES, m/z)⁺: 299 [M+H]⁺.

### Step 3. {[(2s,4r)-6-[3-(pyridin-2-yl)-1,2-oxazole-5-carbonyl]-6-azaspiro[3.4]octan-2-yl]amino}formonitrile and {[(2r,4s)-6-[3-(pyridin-2-yl)-1,2-oxazole-5-carbonyl]-6-azaspiro[3.4]octan-2-yl]amino}formonitrile (45)

To a stirred mixture of 6-[3-(pyridin-2-yl)-1, 2-oxazole-5-carbonyl]-6-azaspiro [3.4]octan-2-amine (150 mg, 0.49 mmol) and NaHCO₃ (441 mg, 5.14 mmol) in DMF (3 mL) was added BrCN (52 mg, 0.48 mmol) in portions at 0° C. The resulting mixture was stirred for 2 hours at room temperature. The mixture was diluted with ice/water (20 mL) and extracted with DCM (3×20 mL). The combined organic layers were washed with brine (20 mL), dried over anhydrous sodium sulfate and concentrated under reduced pressure. The residue was purified by reverse flash chromatography (Column: C18 silica gel, 40 g, 20-35 μm; Mobile phase: water (containing 0.05% NH₄HC03) and ACN (5% to 30% in 20 minutes); Detector: UV 254/220 nm) to afford the racemic product (79 mg) as a white solid.

The racemate was separated by Prep-chiral-HPLC with the following conditions: Column: CHIRAL ART Cellulose-SB, 2×25 cm, 5 μm, SB; Mobile Phase: A, Hex (0.1% IPA) and B, IPA (30% to 30% in 31 min); Detector: 254/220 nm; Rt1: 21.353 minutes, Rt2: 25.222 minutes. The collected fraction was concentrated under vacuum and re-lyophilized to afford {[(2s,4r)-6-[3-(pyridin-2-yl)-1,2-oxazole-5-carbonyl]-6-azaspiro[3.4]octan-2-yl]amino}formonitrile (first eluting isomer, Rt1=21.353 minutes) (14.8 mg, 9%) as a white solid, and {[(2r,4s)-6-[3-(pyridin-2-yl)-1,2-oxazole-5-carbonyl]-6-azaspiro[3.4]octan-2-yl]amino}formonitrile (45) (second eluting isomer, Rt2=25.222 minutes) (36.8 mg, 23%) as a white solid.

First Eluting Isomer ¹H NMR (400 MHz, DMSO-d6) δ (ppm): 8.78-8.75 (m, 1H), 8.11-8.08 (m, 1H), 8.02-7.97 (m, 1H), 7.59-7.57 (m, 1H), 7.48 (d, J=4.4 Hz, 1H), 7.21-7.17 (m, 1H), 3.82-3.77 (m, 3H), 3.58 (s, 1H), 3.52-3.50 (m, 1H), 2.37-2.32 (m, 2H), 1.99-1.90 (m, 4H). LCMS (ES, m/z)⁺: 324 [M+H]⁺.

45 ¹H NMR (400 MHz, DMSO-d6) δ (ppm): 8.76 (d, J=2.8 Hz, 1H), 8.09 (d, J=7.6 Hz, 1H), 8.03-7.99 (m, 1H), 7.59-7.56 (m, 1H), 7.48 (s, 1H), 7.19-7.14 (m, 1H), 3.84-3.81 (m, 1H), 3.75-3.71 (m, 2H), 3.56-3.53 (m, 1H), 3.48 (s, 1H), 2.31-2.24 (m, 2H), 2.07-1.99 (m, 3H), 1.96-1.93 (m, 1H). LCMS (ES, m/z)⁺: 324 [M+H]⁺.

### Example 20

### Synthesis of Additional Embodied Compounds by the Method of Example 17

Additional embodied compounds synthesized using the method of Example 17 are reported in Table A:

TABLE A

| Compound | LCMS (ESI⁺, m/z) |
|---|---|
| Number | [M + H]⁺ |
| 7 | 393.058 |
| 11 | 381.046 |
| 17 | 393.048 |
| 21 | 326.171 |
| 22 | 339.152 |
| 23 | 326.169 |
| 25 | 311.072 |
| 26 | 379.037 |
| 27 | 310.14 |
| 28 | 322.145 |
| 29 | 367.042 |
| 30 | 324.146 |
| 31 | 339.152 |
| 32 | 395.075 |
| 33 | 338.173 |
| 34 | 339.152 |
| 35 | 324.146 |
| 36 | 325.077 |
| 37 | 324.146 |
| 38 | 339.150 |
| 39 | 381.033 |
| 40 | 322.197 |
| 41 | 315.142 |
| 42 | 308.115 |
| 44 | 421.08 |
| 46 | 313.081 |
| 47 | 342.183 |
| 48 | 301.101 |
| 49 | 340.187 |

### Example 21

### TRABID Inhibition Biochemical Assay Protocol

TRABID enzymatic assays were performed in a final volume of 6 μL in buffer containing 20 mM Tris-HCl pH 8.0, (Corning 46-031-CM), and 1 mM GSH (Sigma, G4251), 0.03% BGG (Sigma, G7516), and 0.01% Triton X-100 (Sigma, 93443). Test compounds were serially diluted in DMSO (Sigma, G7516) to obtain 10-point, 3-fold series. Nanoliter quantities were pre-dispensed into 1536 assay plates (Corning, 9110BC) for the concentration response range, 26.6 μM to 1.35 nM. 3 μL of 2× enzyme was added to the assay plates, preincubated with compound for 30 minutes and then 3 μL of 2× substrate was added to initiate the reaction (10 nM TRABID(245-697) and 25 nM Ub-Rh110MP (UbiQ, UbiQ-126) final concentrations). Enzyme and substrate concentrations and incubation times were optimized for the maximal signal-to-background while maintaining linear initial velocity conditions at a fixed substrate concentration below Km.

Fluorescence signal was measured on an EnVision Plate Reader (PerkinElmer) equipped with 485 nm excitation filter and 535 nm emission filters. Measurements were taken at 0, 30 and 60 minutes, curves were shown to progress linearly.

Rates were calculated by: rate=((final FLU−initial FLU)/600 seconds) where final FLU=fluorescence at time 10 minutes, initial FLU=fluorescence at time 0 minutes and 600=duration of reaction in seconds.

Data were reported as percent inhibition compared with control wells based on the following equation: % inh=100*((rate−AveLow)/(AveHigh−AveLow)) where rate=measured rate of fluorescence generated during assay, AveLow=average rate of no enzyme control (n=32), and AveHigh=average rate of DMSO control (n=32).

IC50 values were determined by curve fitting of the standard 4 parameter logistic fitting algorithm included in the Activity Base software package (IDBS) using XE Designer equation Model 205. Data were fitted using the Levenburg Marquardt algorithm. IC50 values for specific embodied compounds are reported in Table B.

TABLE B

| Compound | | TRABID |
|---|---|---|
| Number | Structure and Name | IC₅₀ (μM) |
| 1 | 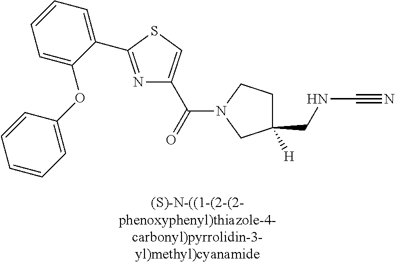 [SMILES: N#CNC[C@@H]1CCN(C(=O)c2csc(-c3ccccc3Oc3ccccc3)n2)C1] | 0.1399 |
| 2 | 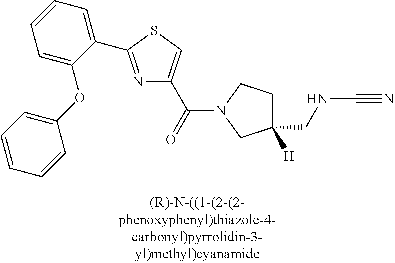 [SMILES: N#CNC[C@H]1CCN(C(=O)c2csc(-c3ccccc3Oc3ccccc3)n2)C1] | 0.439 |
| 3 | 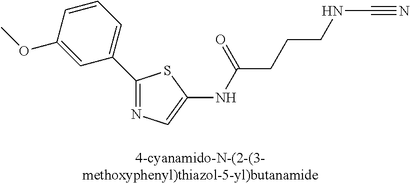 [SMILES: COc1cccc(-c2ncc(NC(=O)CCCNC#N)s2)c1] | 0.743 |
| 4 | 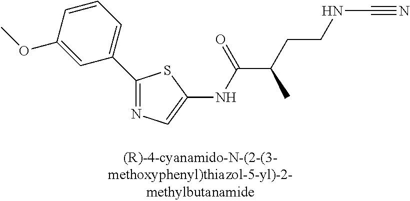 [SMILES: COc1cccc(-c2ncc(NC(=O)[C@H](C)CCNC#N)s2)c1] | 0.85 |
| 5 | 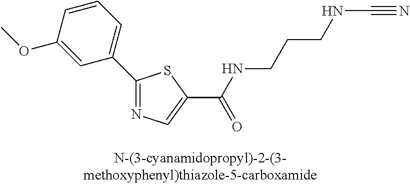 [SMILES: COc1cccc(-c2ncc(C(=O)NCCCNC#N)s2)c1] | 0.872 |
| 6 | 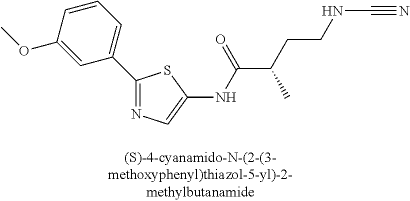 [SMILES: COc1cccc(-c2ncc(NC(=O)[C@@H](C)CCNC#N)s2)c1] | 0.951 |
| 7 | 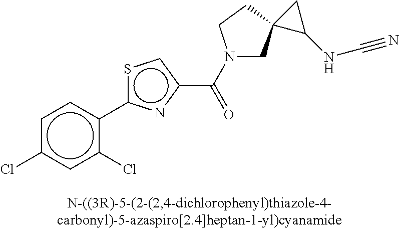 | 1.056 |
| 8 | 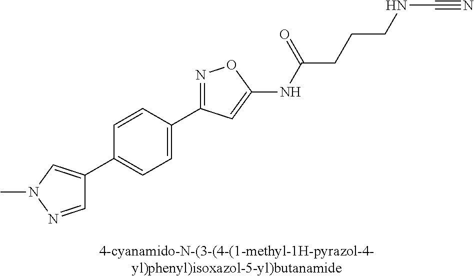 [SMILES: Cn1cc(-c2ccc(-c3cc(NC(=O)CCCNC#N)on3)cc2)cn1] | 1.058 |
| 9 | 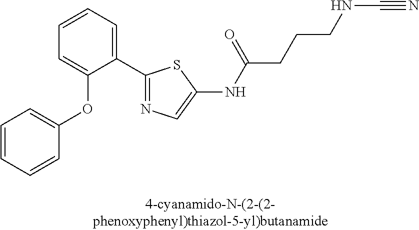 [SMILES: N#CNCCCC(=O)Nc1cnc(-c2ccccc2Oc2ccccc2)s1] | 1.133 |
| 10 | 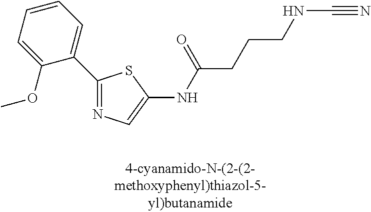 [SMILES: COc1ccccc1-c1ncc(NC(=O)CCCNC#N)s1] | 1.155 |
| 11 | 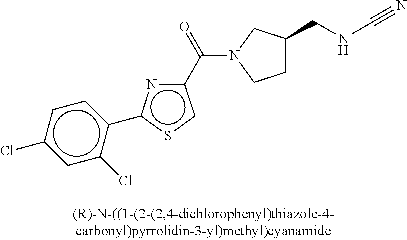 | 1.158 |
| 12 | 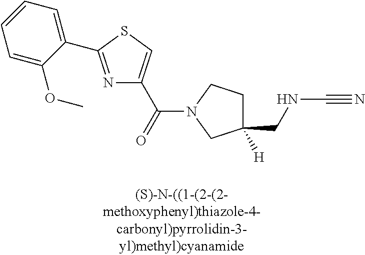 [SMILES: COc1ccccc1-c1nc(C(=O)N2CC[C@@H](CNC#N)C2)cs1] | 1.248 |
| 13 | 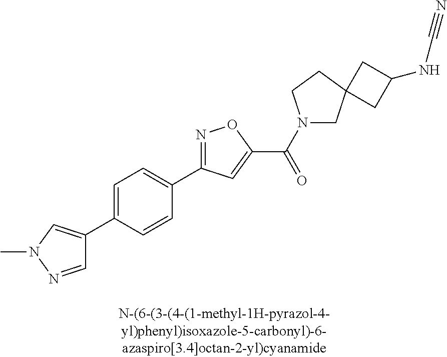 [SMILES: Cn1cc(-c2ccc(-c3cc(C(=O)N4CCC5(CC(NC#N)C5)C4)on3)cc2)cn1] | 1.259 |
| 14 | 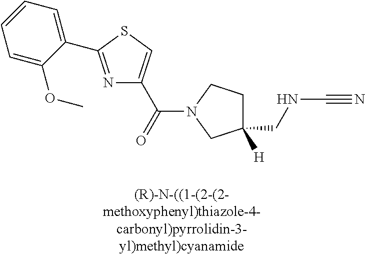 [SMILES: COc1ccccc1-c1nc(C(=O)N2CC[C@H](CNC#N)C2)cs1] | 1.281 |
| 15 | 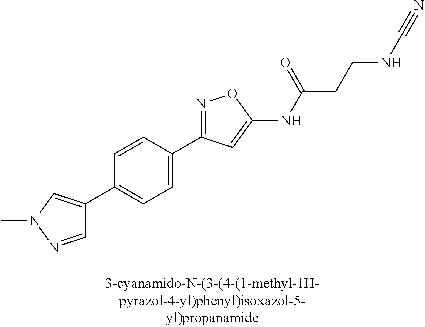 [SMILES: Cn1cc(-c2ccc(-c3cc(NC(=O)CCNC#N)on3)cc2)cn1] | 1.395 |
| 16 | 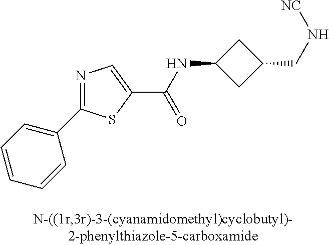 [SMILES: N#CNC[C@H]1C[C@H](NC(=O)c2cnc(-c3ccccc3)s2)C1] | 1.579 |
| 17 | 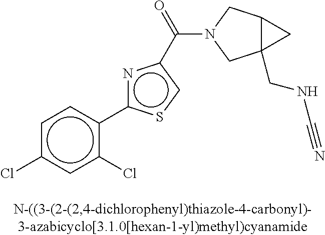 | 1.604 |
| 18 | 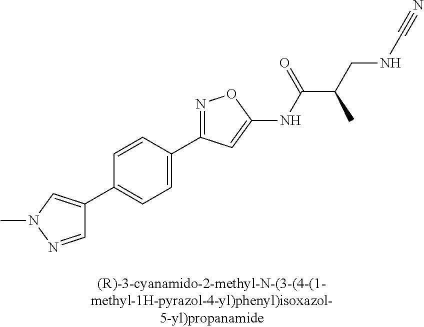 [SMILES: C[C@H](CNC#N)C(=O)Nc1cc(-c2ccc(-c3cnn(C)c3)cc2)no1] | 1.663 |
| 19 | 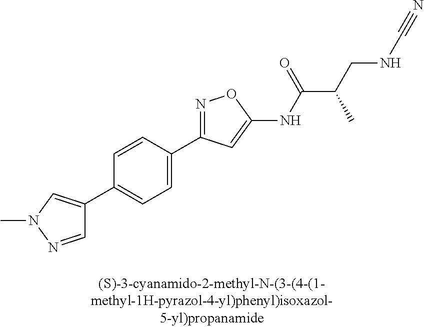 [SMILES: C[C@@H](CNC#N)C(=O)Nc1cc(-c2ccc(-c3cnn(C)c3)cc2)no1] | 2.59 |
| 20 | 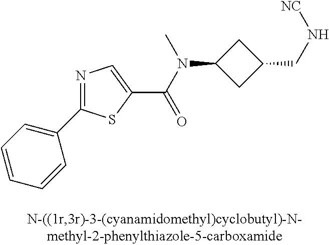 [SMILES: CN(C(=O)c1cnc(-c2ccccc2)s1)[C@H]1C[C@H](CNC#N)C1] | 2.95 |
| 21 | 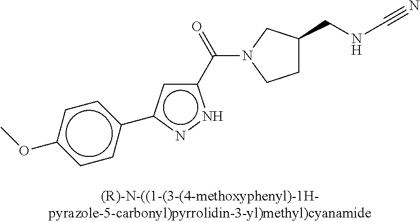 | 2.96 |
| 22 | 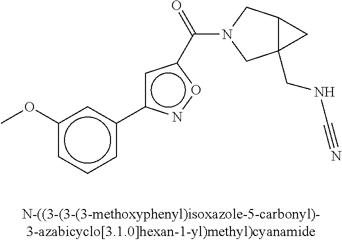 | 3.4 |
| 23 | 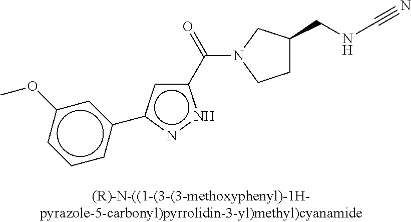 | 3.48 |
| 24 | 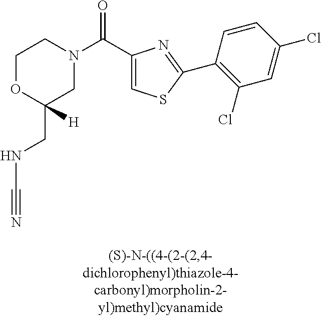 [SMILES: N#CNC[C@H]1CN(C(=O)c2csc(-c3ccc(Cl)cc3Cl)n2)CCO1] | 4 |
| 25 | 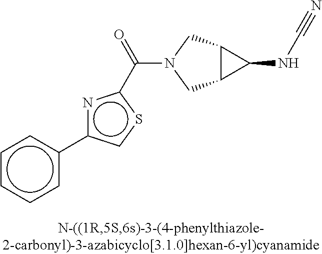 | 4.41 |
| 26 | 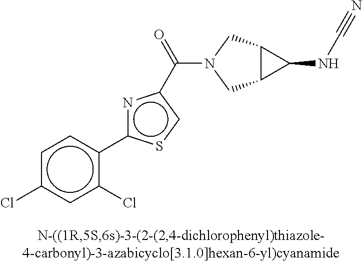 | 4.53 |
| 27 | 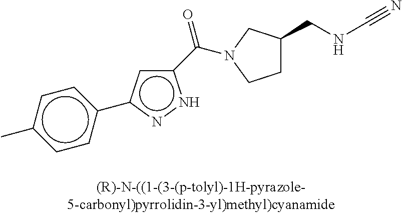 | 4.53 |
| 28 | 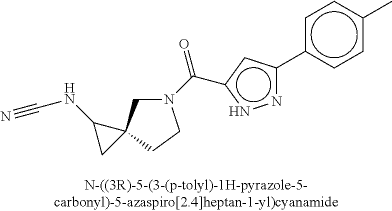 | 5.05 |
| 29 | 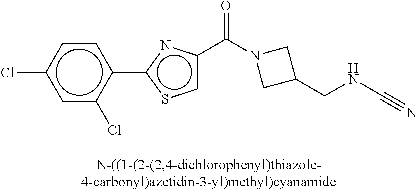 | 5.16 |
| 30 | 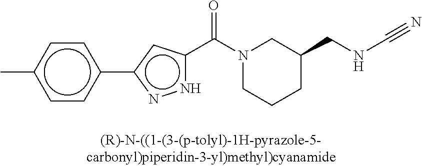 | 5.21 |
| 31 | 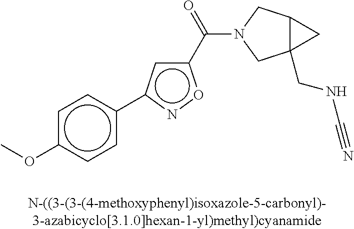 | 5.21 |
| 32 | 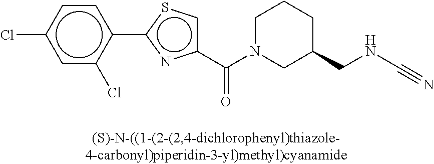 | 5.27 |
| 33 | 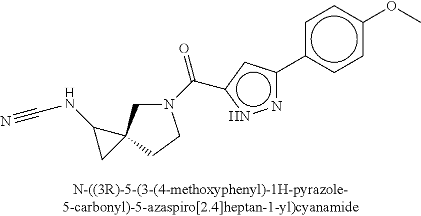 | 5.47 |
| 34 | 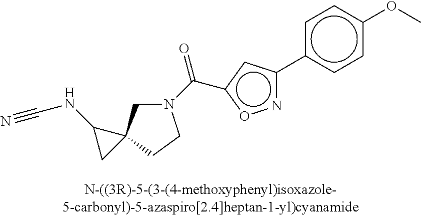 | 5.66 |
| 35 | 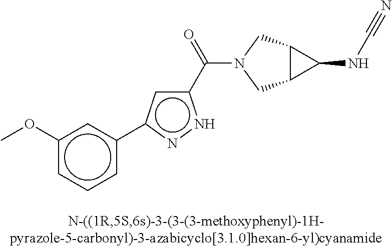 | 6.16 |
| 36 | 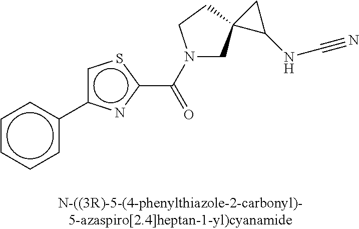 | 6.51 |
| 37 | 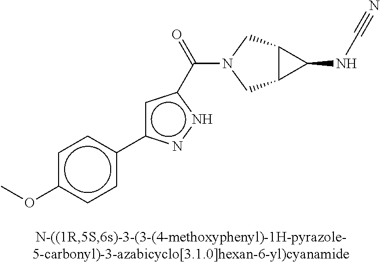 | 6.51 |
| 38 | 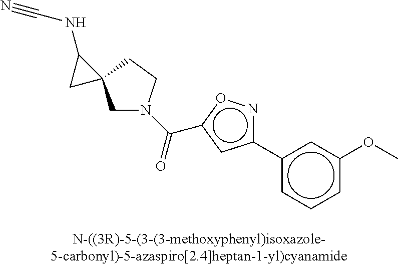 | 0.52 |
| 39 | 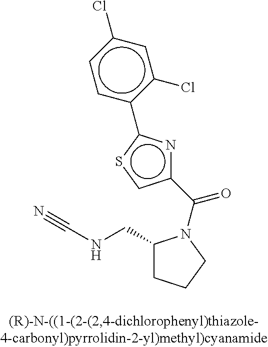 | 6.62 |
| 40 | 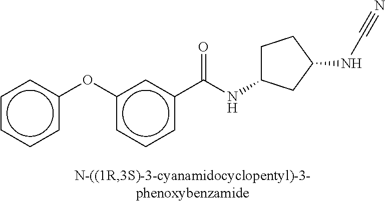 | 7.29 |
| 41 | 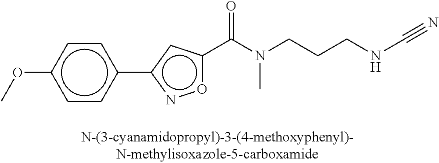 | 7.75 |
| 42 | 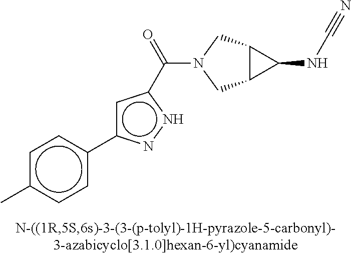 | 7.76 |
| 43 | 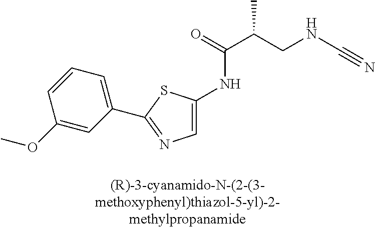 [SMILES: COc1cccc(-c2ncc(NC(=O)[C@H](C)CNC#N)s2)c1] | 7.88 |
| 44 | 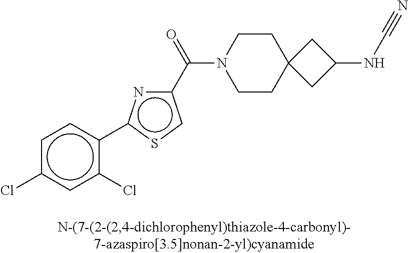 | 8 |
| 45 | 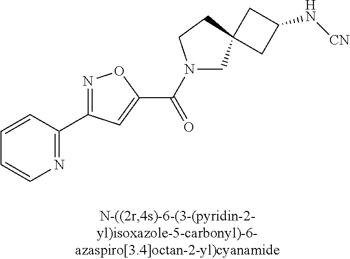 [SMILES: N#CN[C@H]1C[C@]2(CCN(C(=O)c3cc(-c4ccccn4)no3)C2)C1] | 8.1 |
| 46 | 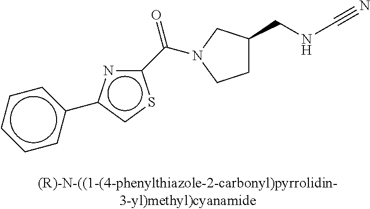 | 8.47 |
| 47 | 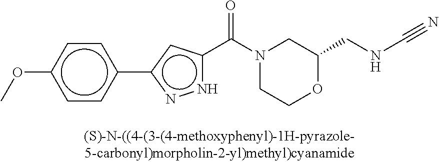 | 8.49 |
| 48 | 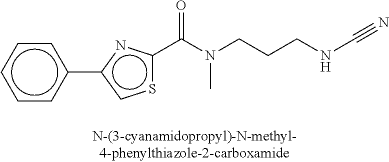 | 8.74 |
| 49 | 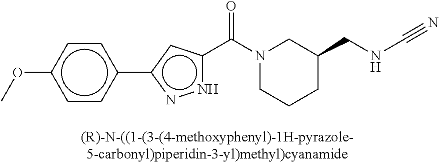 | 8.93 |

### EMBODIMENTS OF THE DISCLOSURE

Embodiment 1. A compound of formula (I) as described herein, or a pharmaceutically acceptable salt thereof.

Embodiment 2. The compound of embodiment 1, wherein z is one.

Embodiment 3. The compound of embodiment 1 or embodiment 2, wherein Ar₁ is selected from the group consisting of: pyrazolyl, thiazolyl, and isoxazolyl.

Embodiment 4. The compound of any one of embodiments 1-3, wherein Ar₁ is unsubstituted pyrazolyl, unsubstituted thiazolyl, or unsubstituted isoxazolyl.

Embodiment 5. The compound of any one of embodiments 1-4, wherein R₁ and R₁′ are each hydrogen.

Embodiment 6. The compound of any one of embodiments 1-4, wherein R₁ forms a heterocyclyl with R₄.

Embodiment 7. The compound of embodiment 6, wherein the heterocyclyl is a 6- to 9-membered heterocyclyl.

Embodiment 8. The compound of embodiment 7, wherein the heterocyclyl is selected from the group consisting of 6-membered fused heterocyclyl and 7- to 9-membered spirocyclyl.

Embodiment 9. The compound of embodiment 7 or embodiment 8, wherein the heterocyclyl is 6-membered fused heterocyclyl.

Embodiment 10. The compound of embodiment 7 or embodiment 8, wherein the heterocyclyl is 7-membered spirocyclyl.

Embodiment 11. The compound of embodiment 7 or embodiment 8, wherein the heterocyclyl is 8-membered spirocyclyl.

Embodiment 12. The compound of embodiment 7 or embodiment 8, wherein the heterocyclyl is 9-membered spirocyclyl.

Embodiment 13. The compound of any one of embodiments 7-12, wherein the heterocyclyl includes 1 heteroatom, the heteroatom being N.

Embodiment 14. The compound of any one of embodiments 1-4, wherein R₁ is (C₁-C₄) alkyl.

Embodiment 15. The compound of any one of embodiment 14, wherein R₁ is methyl.

Embodiment 16. The compound of any one of embodiments 1-5, wherein R₂ and R₂′ are each hydrogen.

Embodiment 17. The compound of any one of embodiments 1-5, wherein R₂ is (C₁-C₄) alkyl.

Embodiment 18. The compound of embodiment 17, wherein R₂ is methyl.

Embodiment 19. The compound of any of embodiments 1-5, wherein R₂ forms a cycloalkyl with R₃.

Embodiment 20. The compound of embodiment 19, wherein the cycloalkyl is a (C₃-C₆) cycloalkyl.

Embodiment 21. The compound of embodiment 19 or embodiment 20, wherein the cycloalkyl is cyclobutyl.

Embodiment 22. The compound of any of embodiments 1-5, or embodiment 6, wherein R₂ forms a heterocycloalkyl with R₄.

Embodiment 23. The compound of embodiment 22, wherein the heterocycloalkyl is a 4- to 6-membered heterocycloalkyl having 1-2 heteroatoms.

Embodiment 24. The compound of embodiment 22 or 23, wherein the heterocycloalkyl is selected from the group consisting of pyrrolidinyl, morpholinyl, azetidinyl, piperidinyl, and fused heterocyclyl.

Embodiment 25. The compound of any one of embodiments 1-5, wherein R₃ and R₃′ are each hydrogen.

Embodiment 26. The compound any one of embodiments 1-5, wherein R₃ is (C₁-C₄) alkyl.

Embodiment 27. The compound of embodiment 26, wherein R₃ is methyl.

Embodiment 28. The compound of any one of embodiments 1-5, wherein R₄ is hydrogen.

Embodiment 29. The compound of any one of embodiments 1-5, wherein R₄ is (C₁-C₄) alkyl.

Embodiment 30. The compound of embodiment 29, wherein R₄ is methyl.

Embodiment 31. The compound of any one of embodiments 1-5, wherein L is —C(O)NR₄, and Ar₂ is phenyl substituted with one or more R₁₀ or R₁₂.

Embodiment 32. The compound of embodiment 31, wherein R₁₀ is selected from halogen, (C₁-C₆) alkyl, (C₁-C₆) alkoxy, and aryloxy.

Embodiment 33. The compound of embodiment 32, wherein R₁₀ is halogen.

Embodiment 34. The compound of embodiment 33, wherein R₁₀ is Cl.

Embodiment 35. The compound of embodiment 32, wherein R₁₀ is (C₁-C₆) alkyl.

Embodiment 36. The compound of embodiment 35, wherein R₁₀ is methyl.

Embodiment 37. The compound of embodiment 32, wherein R₁₀ is (C₁-C₆) alkoxy.

Embodiment 38. The compound of embodiment 37, wherein R₁₀ is methoxy.

Embodiment 39. The compound of embodiment 32, wherein R₁₂ is aryloxy.

Embodiment 40. The compound of embodiment 39, wherein R₁₂ is phenoxy.

Embodiment 41. The compound of any one of embodiments 1-5, wherein L is —NR₄C(O)—, and Ar₂ is unsubstituted phenyl.

Embodiment 42. The compound of any one of embodiments 1-5, wherein L is —NR₄C(O)—, and Ar₂ is unsubstituted pyridinyl.

Embodiment 43. The compound of any one of embodiments 1-5, wherein L is —NR₄C(O)—, and Ar₂ is phenyl substituted with one or more R₁₀ or R₁₂.

Embodiment 44. The compound of embodiment 43 wherein R₁₀ is selected from halogen, (C₁-C₆) alkoxy, and aryloxy.

Embodiment 45. The compound of embodiment 44, wherein R₁₀ is halogen.

Embodiment 46. The compound of embodiment 45, wherein R₁₀ is Cl.

Embodiment 47. The compound of embodiment 44, wherein R₁₀ is (C₁-C₆) alkoxy.

Embodiment 48. The compound of embodiment 47, wherein R₁₀ is methoxy.

Embodiment 49. The compound of embodiment 44, wherein R₁₂ is aryloxy.

Embodiment 50. The compound of embodiment 49, wherein R₁₂ is phenoxy.

Embodiment 51. The compound of any one of embodiments 1-4, wherein the compound is further given by formula (II):

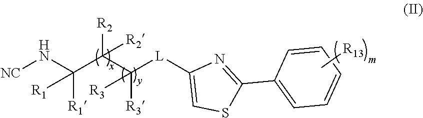

and pharmaceutically acceptable salts thereof, wherein:
L is —NR₄C(O)—;
R₁₃ is selected from halogen, (C₁-C₄) alkoxy, and aryloxy; and
m is 1 or 2.

Embodiment 52. The compound of any one of embodiments 1-4 and embodiment 51, wherein R₁₃ is selected from the group consisting of: —Cl, —OCH₃, and —OC₆H5.

Embodiment 53. The compound of e any one of embodiments 1-4 and embodiments 51-52, selected from the group consisting of:

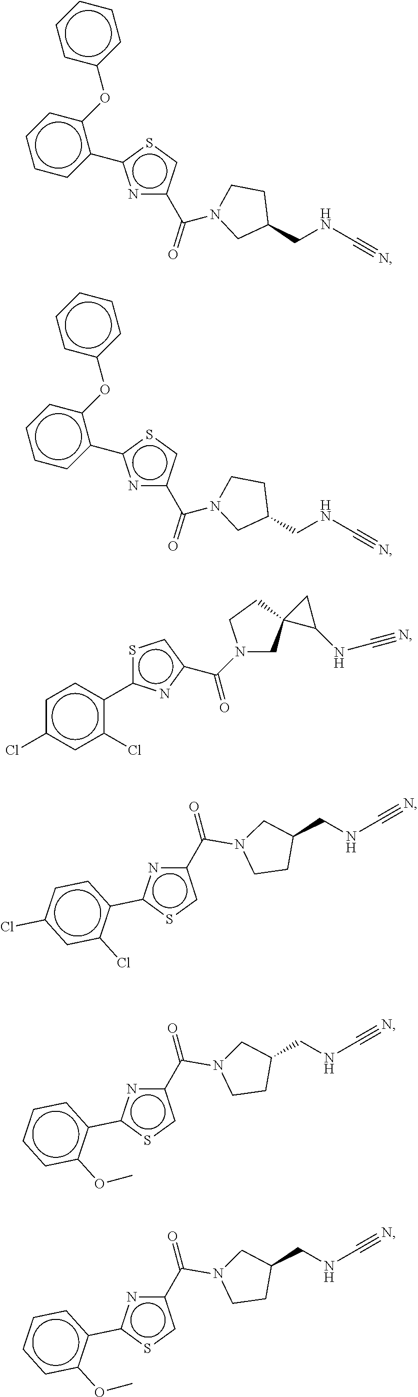

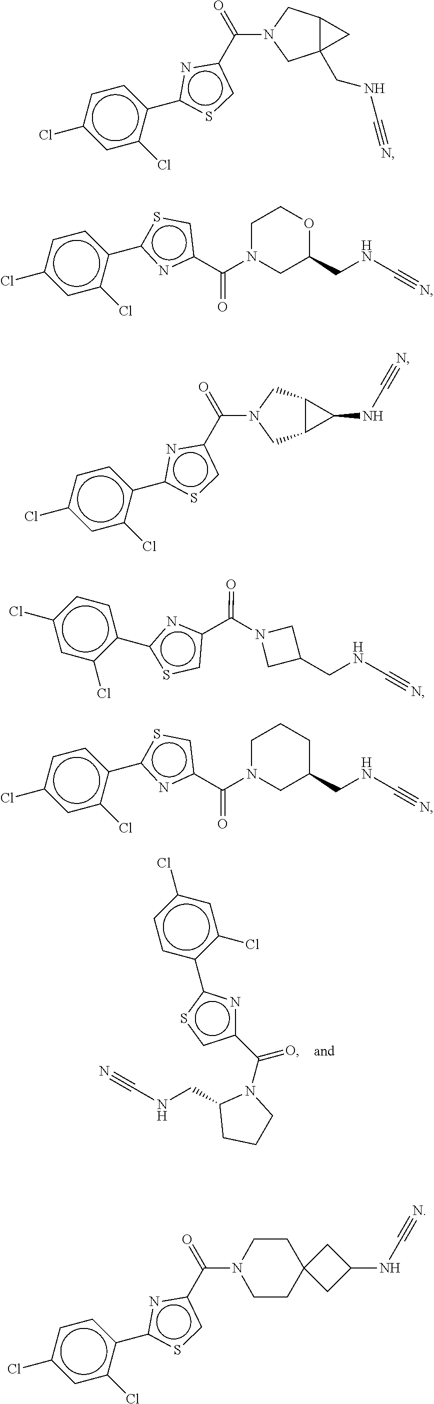

Embodiment 54. The compound of any one of embodiments 1-4, wherein the compound is further given by formula (III):

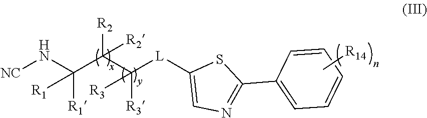

and pharmaceutically acceptable salts thereof, wherein R₁₄ is (C₁-C₄) alkoxy or aryloxy;
and n is 0 or 1.

Embodiment 55. The compound of any one of embodiments 1-4 and embodiment 54, wherein R₁₄ is —OCH₃ or —OC₆H5.

Embodiment 56. The compound of any one of embodiments 1-4 embodiment 54-55, selected from the group consisting of:

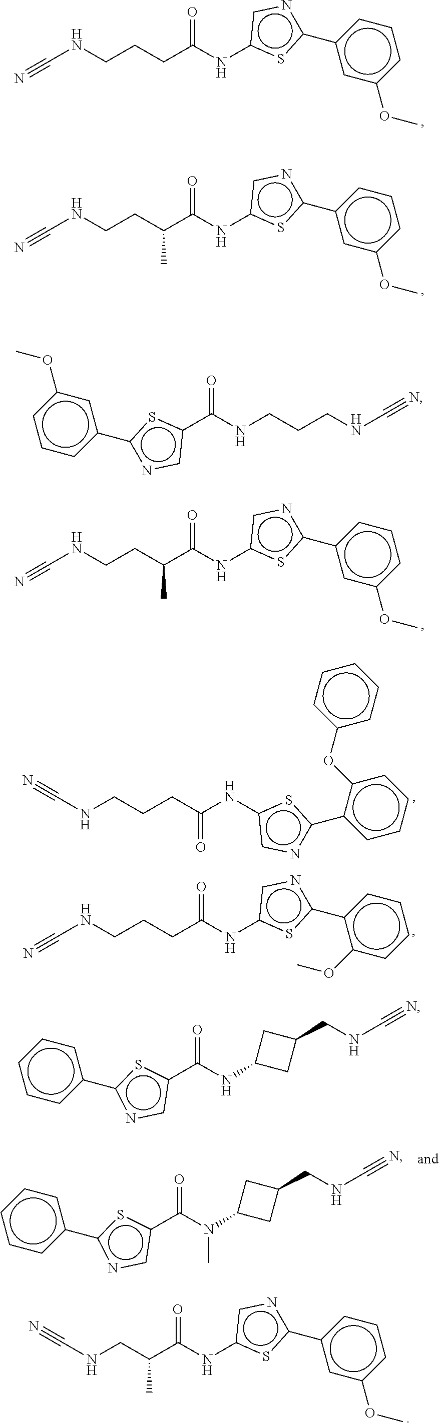

Embodiment 57. The compound of any one of embodiments 1-4, wherein the compound is further given by formula (IV):

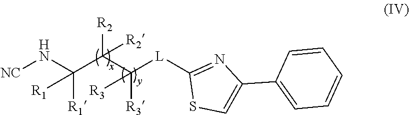

and pharmaceutically acceptable salts thereof, wherein L is —NR₄C(O).

Embodiment 58. The compound of any one of embodiments 1-4 and embodiment 57, selected from the group consisting of:

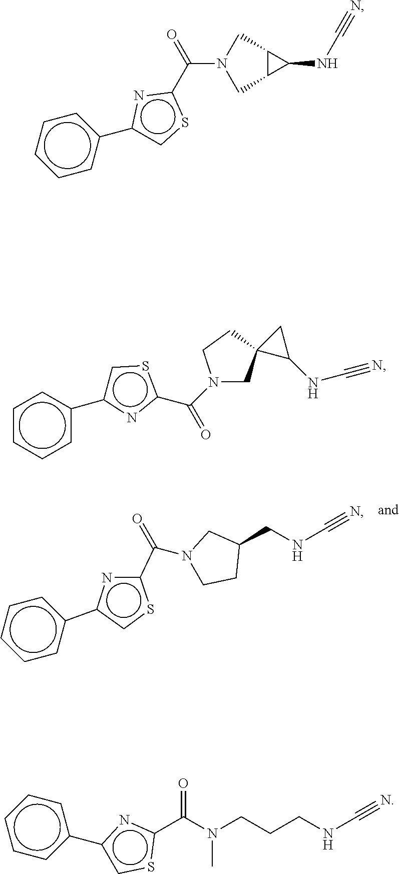

Embodiment 59. The compound of any one of embodiments 1-4, wherein the compound is further given by formula (V):

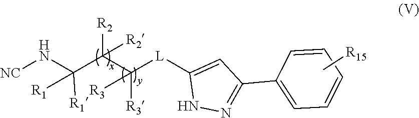

and pharmaceutically acceptable salts thereof, wherein L is —NR₄C(O); and R₁₅ is (C₁-C₄) alkyl or (C₁-C₄) alkoxy.

Embodiment 60. The compound of any one of embodiments 1-4 and embodiment 59, wherein R₁₅ is methyl or —OCH₃.

Embodiment 61. The compound of any one of embodiments 1-4 and embodiment 59-60, selected from the group consisting of:

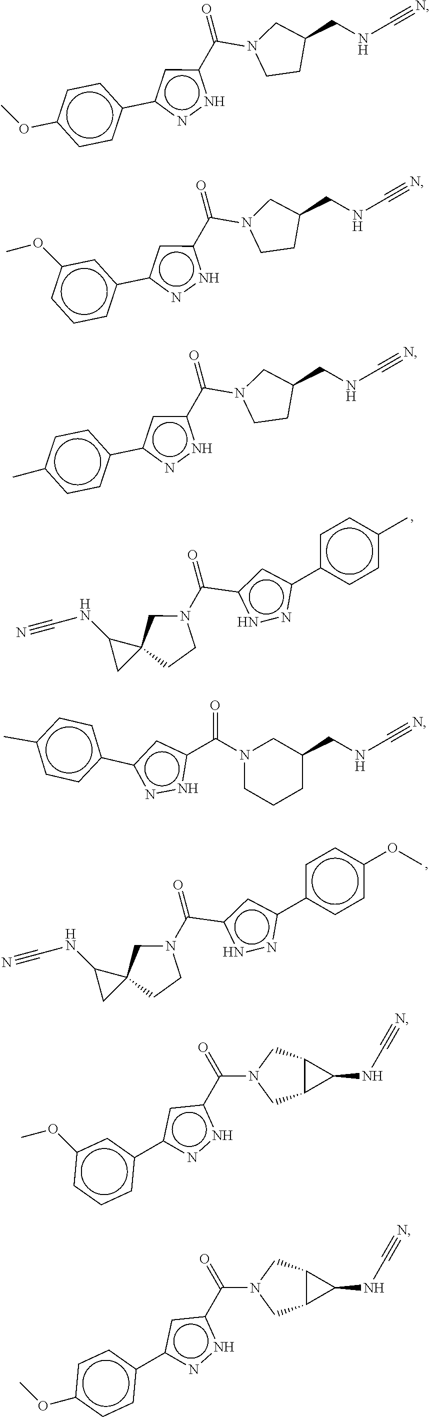

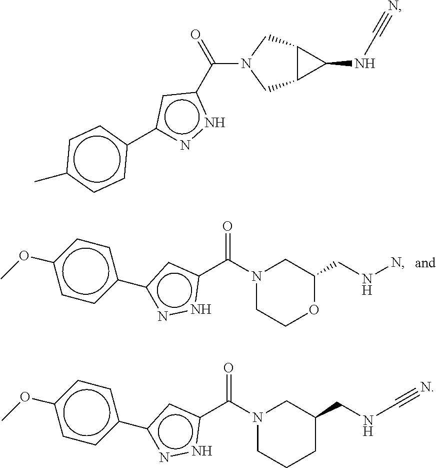

Embodiment 62. The compound of any one of embodiments 1-4, wherein the compound is further given by formula (VI):

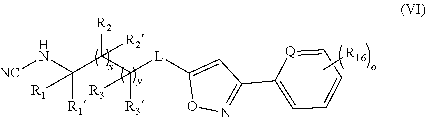

and pharmaceutically acceptable salts thereof, wherein:
Q is N or CH;
R₁₆ is (C₁-C₄) alkoxy or heteroaryl substituted with (C₁-C₄) alkyl; and
is 0 or 1.

Embodiment 63. The compound of any one of embodiments 1-4 and embodiment 62, wherein R₁₆ is —OCH₃ or pyrazolyl substituted with one methyl.

Embodiment 64. The compound of embo any one of embodiments 1-4 and embodiments 62-63, selected from the group consisting of:

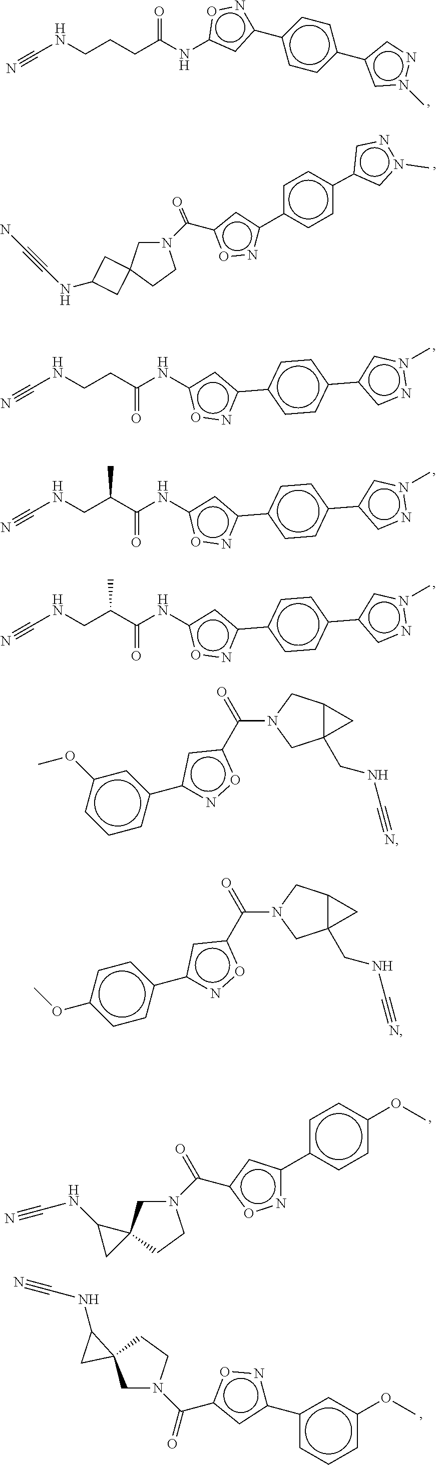

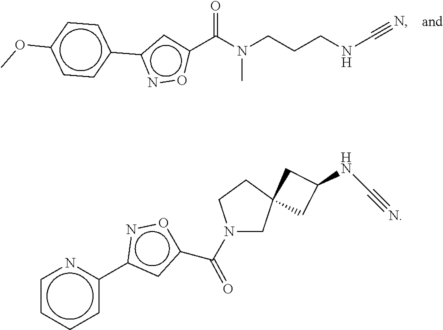

Embodiment 65. The compound of embodiment 1, wherein z is zero.

Embodiment 66. The compound of embodiment 1 or embodiment 65, wherein the compound is further given by formula (VII):

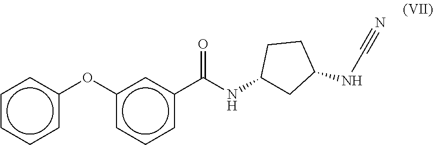

and pharmaceutically acceptable salts thereof.

Embodiment 67. A pharmaceutical composition comprising the compound of any one of embodiments 1-66 and one or more of a pharmaceutically acceptable carrier, adjuvant, or vehicle.

Embodiment 68. A method of inhibiting TRABID in a patient comprising administering to the patient in need thereof, an effective amount of the compound of any one of embodiments 1-66.

Embodiment 69. A method of inhibiting TRABID in a patient comprising administering to the patient in need thereof, an effective amount of the pharmaceutical composition of embodiment 67.

Embodiment 70. A method of treating, preventing, inhibiting, or eliminating a disease or disorder associated with the activity of TRABID in a patient comprising: administering to the patient in need thereof, a therapeutically effective amount of the compound of any one of embodiments 1-66.

Embodiment 71. A method of treating, preventing, inhibiting, or eliminating a disease or disorder associated with the activity of TRABID in a patient comprising: administering to the patient in need thereof, a therapeutically effective amount of the pharmaceutical composition of embodiment 67.

Embodiment 72. The method of embodiment 70, wherein the disease or disorder is an autoimmune inflammatory disease.

Embodiment 73. The method of embodiment 71, wherein the disease or disorder is an autoimmune inflammatory disease.

Embodiment 74. The disease or disorder of embodiment 72, wherein the autoimmune inflammatory disease is psoriasis.

Embodiment 75. The disease or disorder of embodiment 73, wherein the autoimmune inflammatory disease is psoriasis.

The foregoing Description and Examples are exemplary of the present invention and not limiting thereof. The scope of the invention is therefore set out in the appended claims.

1. A compound of formula (I): 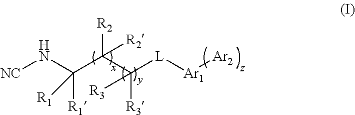
and pharmaceutically acceptable salts thereof, wherein z is zero or one, and
(A) when z is one:
R₁ and R₁′ are each independently hydrogen, (C₁-C₄) alkyl, or together with one of R₂, R₃, or R₄, form a heterocycloalkyl;
R₂ and R₂′ are each independently hydrogen, (C₁-C₄) alkyl, or together with one of R₁, R₃, or R₄, form a cycloalkyl or heterocycloalkyl;
R₃ and R₃′ are each independently hydrogen, (C₁-C₄) alkyl, or together with one of R₁, R₂, or R₄, form a cycloalkyl or heterocycloalkyl;
x and y are each independently zero or one;
L is —C(O)NR₄— or —NR₄C(O)—;
R₄ is hydrogen, (C₁-C₆) alkyl, or together with any one of R₁, R₂, or R₃, form a heterocycloalkyl;
Ar₁ is unsubstituted heteroaryl with up to two heteroatoms independently selected from the group consisting of N, O, and S;
Ar₂ is independently an aryl or heteroaryl optionally substituted with one or more R₁₀, R₁₂, or —OR₁₂, or together with Ar₁, form a fused bicyclic (C₈-C₁₀) aryl or heteroaryl optionally substituted with one or more R₁₁;
each R₁₀ is independently halogen, (C₁-C₆) alkyl, (C₁-C₆) cycloalkyl, (C₁-C₆) alkoxy, aryloxy, or aryl or heteroaryl optionally substituted with one or more R₁₁;
each R₁₁ is independently hydroxyl or halogen; and
R₁₂ is aryl or heteroaryl, optionally substituted with one or more R₁₀;
(1) wherein, when L is —C(O)NR₄—, x is one;
R₁, R₁′, R₂′, and R₄ are each hydrogen;
R₂ is hydrogen or (C₁-C₄) alkyl;
R₃, if present, is hydrogen or (C₁-C₄) alkyl;
R₃′, if present, is hydrogen; and
no combination of R₁, R₁′, R₂, R₂′, R₃, R₃′, and R₄ forms a cycloalkyl or heterocycloalkyl;
(2) wherein, when L is —NR₄C(O)—,
(a) when an R₁₀ or R₁₂ is heteroaryl substituted with alkyl, and at least two of R₁, R₁′, R₂, R₂′, R₃, R₃′, and R₄ combine to define a spirocyclyl comprising (i) a pyrrolidinyl and a cyclobutyl having a carbon atom as a spiro atom, or (ii) an azetidinyl and a cyclobutyl having a carbon atom as a spiro atom, the spiro atom is not adjacent a nitrogen of the pyrrolidinyl or the azetidinyl;
(b) when Ar₂ is substituted with more than one halogen, and R₄ forms a 5- or 6-membered heterocyclyl with another substituent, R₁ and R₁′ are each hydrogen;
(c) when Ar₂ is unsubstituted phenyl, and one of R₂, R₃, and R₄ forms a cyclobutyl or a spirocyclyl which includes a cyclobutyl, R₁ and R₁′ are each hydrogen;
(B) when z is zero:
R₁ and R₁′ are each independently hydrogen, (C₁-C₄) alkyl, or together with one of R₂, R₃, or R₄, form a cycloalkyl or heterocycloalkyl;
R₂ and R₂′ are each independently hydrogen, (C₁-C₄) alkyl, or together with one of R₁, R₃, or R₄, form a cycloalkyl or heterocycloalkyl;
R₃ and R₃′ are each independently hydrogen, (C₁-C₄) alkyl, or together with one of R₁, R₂, or R₄, form a cycloalkyl or heterocycloalkyl;
x and y are each independently zero or one;
L is —C(O)NR₄— or —NR₄C(O)—;
R₄ is hydrogen, (C₁-C₆) alkyl, or together with any one of R₁, R₂, or R₃, form a heterocycloalkyl; and
Ar₁ is independently an aryl or heteroaryl substituted with one aryloxy.

2. The compound of claim 1, wherein z is one.

3. The compound of claim 1, wherein Ar₁ is selected from the group consisting of: pyrazolyl, thiazolyl, and isoxazolyl.

4. The compound of claim 1, wherein Ar₁ is unsubstituted pyrazolyl, unsubstituted thiazolyl, or unsubstituted isoxazolyl.

5. The compound of claims 1-4, wherein the compound is further given by formula (II): 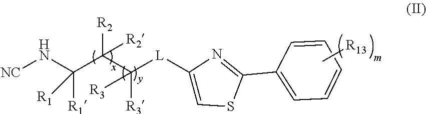
and pharmaceutically acceptable salts thereof, wherein L is —NR₄C(O)—; R₁₃ is selected from halogen, (C₁-C₄) alkoxy, and aryloxy; and m is 1 or 2.

6. The compound of claim 1, wherein R₁₃ is selected from the group consisting of: —Cl, —OCH₃, and —OC₆H₅.

7. (canceled)

8. The compound of claim 1, wherein the compound is further given by formula (III): 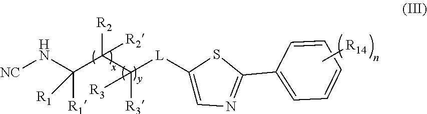
and pharmaceutically acceptable salts thereof, wherein R₁₄ is (C₁-C₄) alkoxy or aryloxy; and n is 0 or 1.

9. The compound of claim 1, wherein R₁₄ is —OCH₃ or —OC₆H₅.

10. (canceled)

11. The compound of claim 1, wherein the compound is further given by formula (IV): 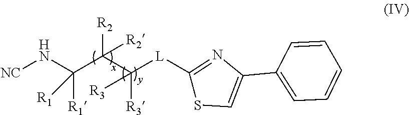
and pharmaceutically acceptable salts thereof, wherein L is —NR₄C(O).

12. (canceled)

13. The compound of claim 1, wherein the compound is further given by formula (V): 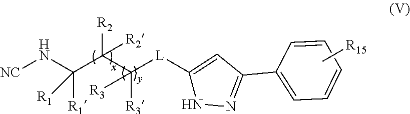
and pharmaceutically acceptable salts thereof, wherein L is —NR₄C(O); and R₁₅ is (C₁-C₄) alkyl or (C₁-C₄) alkoxy.

14. The compound of claim 1, wherein R₁₅ is methyl or —OCH₃.

15. (canceled)

16. The compound of claim 1, wherein the compound is further given by formula (VI): 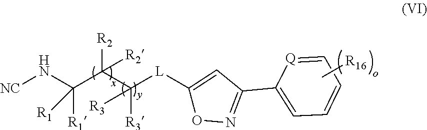
and pharmaceutically acceptable salts thereof, wherein Q is N or CH; R₁₆ is (C₁-C₄) alkoxy or heteroaryl substituted with (C₁-C₄) alkyl; and o is 0 or 1.

17. The compound of claim 1, wherein R₁₆ is —OCH₃ or pyrazolyl substituted with one methyl.

18. (canceled)

19. The compound of claim 1, wherein z is zero.

20. The compound of claim 1, wherein the compound is further given by formula (VII): 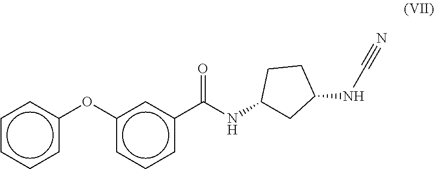
and pharmaceutically acceptable salts thereof.

21. A pharmaceutical composition comprising the compound of claim 1 and one or more of a pharmaceutically acceptable carrier, adjuvant, or vehicle.

22. A method of inhibiting TRABID in a patient comprising administering to the patient in need thereof, an effective amount of the compound of claim 1.

23. A method of inhibiting TRABID in a patient comprising administering to the patient in need thereof, an effective amount of the pharmaceutical composition of claim 21.

24. A method of treating, preventing, inhibiting, or eliminating a disease or disorder associated with the activity of TRABID in a patient comprising:
administering to the patient in need thereof, a therapeutically effective amount of the compound of claim 1.

25. (canceled)

26. The method of claim 24, wherein the disease or disorder is an autoimmune inflammatory disease.

27. (canceled)
